(19) United States
(12) Patent Application Publication
(10) Pub. No.: US 2009/0155255 A1
(43) Pub. Date: Jun. 18, 2009

(54) CD23 BINDING MOLECULES AND METHODS OF USE THEREOF

(75) Inventors: Scott Glaser, San Diego, CA (US); Brian Robert Miller, San Diego, CA (US); Alexey Alexandrovich Lugovskoy, Woburn, MA (US); Stephen Demarest, San Diego, CA (US); Ann Maclaren, San Diego, CA (US); William B. Snyder, Escondido, CA (US)

Correspondence Address: LAHIVE & COCKFIELD, LLP / BIOGEN IDEC;FLOOR 30, SUITE 3000, ONE POST OFFICE SQUARE, BOSTON, MA, 02109-2127, US

(73) Assignee: Biogen Idec MA Inc., Cambridge, MA (US)

(21) Appl. No.: 12/240,880

(22) Filed: Sep. 29, 2008

Related U.S. Application Data
(60) Provisional application No. 60/995,747, filed on Sep. 27, 2007.

(51) Int. Cl. A61K 39/395; C07K 16/18; C12N 15/11; C12N 5/06; C12P 21/08

(52) U.S. Cl. 424/133.1; 530/387.1; 530/387.3; 536/23.53; 435/328; 435/69.6; 435/375

(57) ABSTRACT

The invention is based, at least in part, on the development of multivalent and stabilized forms of CD23 binding molecules and methods of use thereof for the treatment of immune cell disorders, including leukemias or lymphomas such as CLL.

### RELATED APPLICATIONS

This application claims benefit under § 119(e) of U.S. Provisional Application No. 60/995,747 filed Sep. 27, 2007, entitled, “CD23 Binding Molecules and Methods of Use Thereof”. The above-referenced patent application is hereby incorporated by reference in its entirety.

### BACKGROUND OF THE INVENTION

Chronic lymphocytic leukemia (also called CLL) is a progressive B-cell disease in which the bone marrow makes functionally incompetent lymphocytes that accumulate in the blood and can spread to the lymph nodes, spleen, liver, and other parts of the body. It is the most common form of leukemia found in adults in Western countries. The cells of origin in the majority of patients with CLL are clonal B cells arrested in the B-cell differentiation pathway, intermediate between pre-B cells and mature B cells. Morphologically in the peripheral blood, these cells resemble mature lymphocytes. B-CLL lymphocytes typically show B-cell surface antigens, as demonstrated by CD5, CD19, CD20, CD21, CD23 and CD24 monoclonal antibodies.

CD23 is a 45-kD type II homotrimeric membrane protein and is the low affinity IgE receptor (FceRII). It is present on many different cell types, including B cells, activated T cells, monocytes, eosinophils, platelets, follicular dendritic cells, and some thymic epithelial cells, where it is thought to play a crucial role in the capture of antigen by specific IgE on antigen-presenting cells. CD23 is constitutively and highly expressed on B-chronic lymphocytic leukemia cells.

Several CD23 binding molecules have shown promise as therapeutics in the treatment of CLL. For example, p5E8 (IDEC 152) is a primatized monoclonal antibody that binds with high affinity to human CD23 (see U.S. Pat. Nos. 7,223,392 and 7,332,163). In small clinical studies p5E8 was show to have synergistic activity in combination with standard chemotherapeutic agents forming the basis for a registrational global, multicenter clinical trial comparing p5E8 with Fludarabine, Cyclophosphamide, and Rituximab (FCR) versus FCR alone in subjects with relapsed CLL.

While these and other studies point to the promise of CD23 binding molecules for treating CLL and form the basis for ongoing clinical development, there remains room for improving the potency and efficacy of CD23 binding molecules as therapeutics for this difficult to treat disease. One promising class of biologics that may improve upon treatment potency and efficacy are multivalent and bispecific binding molecules capable of crosslinking two or more B-cell markers. Although certain such molecules have been reported, these molecules generally rely on cross-linking of whole IgG antibodies via artificial disulfide bonds or other crosslinkers. Accordingly, these molecules may suffer from sub-optimal yields of dimerized antibody and rely on reducing agents to facilitate disulfide bond formation. Moreover, instability in the variable regions of some multivalent or bispecific antibodies may result in a variety of production problems, including one or more of: unsuitability for scale-up production in bioreactors (e.g., because of low yield, significant levels of unwanted byproducts such as unassembled product, and/or aggregated material), difficulties in protein purification, and unsuitability for pharmaceutical preparation and use (e.g., owing to significant levels of breakdown product, poor product quality, and/or unfavorable pharmacokinetic properties). In fact, protein stability is now recognized as a central issue for the development and scale up of many therapeutic proteins and other biologics. Consequently, in certain instances, CD23 binding molecules (particularly multivalent and bispecific CD23 binding molecules) with improved protein stability may be desirable.

### SUMMARY OF THE INVENTION

The invention is based, at least in part, on the development of improved CD23 polypeptide compositions, e.g. improved CD23 binding molecules, having improved potency, efficacy, and/or stability.

In certain aspects, the invention provides a multivalent CD23 binding molecule comprising at least three binding sites and at least two polypeptide chains wherein said binding molecule specifically crosslinks at least two distinct human CD23 molecules (e.g., three, four, or five human CD23 molecules) on an immune cell and enhances CD23-mediated receptor signaling, thereby inducing apoptosis of the immune cell to a greater extent than a bivalent CD23 monoclonal antibody (e.g., a conventional IgG mAb) or a combination of two or more bivalent CD23 mAbs. Preferably, the multivalent CD23 binding molecule induces apoptosis of the immune cell to a greater extent than a dimer formed by crosslinking of two bivalent CD23 monoclonal antibodies with a cross-linker.

In one embodiment, the immune cell is a B cell lymphoma. In another embodiment, the immune cell is a CLL B-cell. In another embodiment, the binding sites are independently selected from (i) an antigen binding site; (ii) an antibody fragment; (iii) a conventional scFv molecule; and (iv) a stabilized scFv molecule.

In another embodiment, the binding molecule is a multivalent CD23 binding molecule comprising at least four binding sites specific for a CD23 molecule and at least two polypeptide chains.

In another embodiment, the binding molecule is a multivalent CD23 binding molecule comprising at least four binding sites specific for a CD23 molecule and at least two polypeptide chains, where at least two of the binding sites are antigen binding sites derived from a CD23 antibody, and at least two of the binding sites are scFv molecules. In one embodiment, the said scFv molecules are stabilized scFv molecules.

In certain embodiments, the binding sites have the same binding specificity. In other embodiments, at least two of the binding sites have different binding specificities. In one embodiment, the binding sites bind different CD23 epitopes on the same CD23 molecule. In another embodiment, the binding sites bind different CD23 molecules. In another embodiment, the binding sites bind different CD23 epitopes on different CD23 molecules.

In one embodiment, the binding molecule is trivalent. In another embodiment, the binding molecule is tetravalent. In another embodiment, the binding molecule is pentavalent. In another embodiment, the binding molecule is hexavalent. In another embodiment, the binding molecule is decavalent.

In other embodiments, at least two of the binding sites are derived from an antibody selected from the group consisting of a chimeric antibody, a humanized antibody, a fully human antibody, and a primatized antibody. In other embodiments, the binding molecule comprises a primatized binding site comprising variable regions from a non-human primate and constant regions from a human.

In other embodiments, at least two of said binding sites are derived from an antibody selected from the group consisting of a 5E8 antibody, a 6G5 antibody, a 2C8 antibody, a B3B11 antibody, and a 3G12 antibody. In other embodiments, at least four of said binding sites are derived from an antibody selected from the group consisting of a 5E8 antibody, a 6G5 antibody, a 2C8 antibody, a B3B11 antibody, and a 3G12 antibody. In other embodiments, all of said binding sites are derived from an antibody selected from the group consisting of a 5E8 antibody, a 6G5 antibody, a 2C8 antibody, a B3B11 antibody, and a 3G12 antibody.

In one embodiment, the multivalent binding molecule is a scFv tetravalent binding molecule. In other embodiments, the multivalent binding molecule is a scFv2 tetravalent binding molecule. In one embodiment, the binding molecule is a N_{L}-scFv tetravalent antibody molecule. In other embodiments, the binding molecule is a N_{H}-scFv tetravalent antibody molecule. In other embodiments, the binding molecule is a C-scFv tetravalent antibody molecule. In other embodiments, at least two of said binding sites form a diabody.

In other embodiments, a binding molecule of the invention comprises an Fc portion, e.g., an Fc portion derived from an IgG1 antibody. In other embodiments, the Fc portion imparts at least one effector function to the binding molecule. In one embodiment, the Fc portion is capable of binding an Fcγ receptor to initiate ADCC of the immune cell. In other embodiments, the Fc portion comprises a complete Fc region. In other embodiments, the Fc portion comprises a chimeric hinge. In other embodiments, the Fc portion is derived from a human antibody of the IgG1 subclass. In other embodiments, the Fc portion is derived from a human antibody of the IgG4 subclass. In other embodiments, the Fc portion comprises at least one Fc variation.

In another aspect, the invention provides a multivalent CD23 binding molecule comprising more than two CD23 binding moieties, wherein said binding molecule specifically crosslinks at least two distinct human CD23 molecules on the surface of an immune cell, thereby inducing apoptosis of the immune cell. In one embodiment, the binding molecule comprises at least four binding moieties. In another embodiment, said binding molecule binds to FcγR. In another embodiment, said binding molecule induces CD23-mediated caspase-3 and PARP cleavage.

In one embodiment, said binding molecule induces apoptosis to a greater extent than an equimolar amount of an antibody dimer formed by crosslinking of two bivalent CD23 monoclonal antibodies with a cross-linker. In another embodiment, the binding molecule induces apoptosis 1.5 fold or more, 2-fold or more, 3-fold or more, 4-fold or more, 5-fold or more, 6 fold or more, 7-fold or more, 8 fold or more, 9-fold or more, 10-fold or more, and 15-fold or more than an equimolar amount of an antibody dimer formed by crosslinking of two bivalent CD23 monoclonal antibodies with a cross-linker. In another embodiment, the equimolar amount is an amount selected from the group consisting of 1 ug/ml or more, 2 ug/ml or more, 5 ug/ml or more, 10 ug/ml or more, 15 ug/ml or more, and 20 ug/ml or more.

In another embodiment, three, four, five, six, seven, eight, nine, ten, or more CD23 molecules are crosslinked by said multivalent CD23 binding molecule.

In one embodiment, apotosis of the immune cell is determined by an apoptotic assay selected from the group consisting of: a PARP cleavage assay, a TUNEL assay, a Caspase cleavage assay, and a mitochondrial membrane permeabilization assay.

In one embodiment, the multivalent binding molecule is not crosslinked to a second multivalent binding molecule by a crosslinker. In another embodiment, the multivalent binding molecule is crosslinked with a second multivalent binding molecule by a crosslinker. In one embodiment, the cross-linker is an antibody which binds to the anti-CD23 antibody. In another embodiment, the cross-linker is an engineered disulfide bond.

In one embodiment, the binding molecule binds to human CD23. In one embodiment, two of said binding moieties are binding sites derived from an antibody selected from the group consisting of a 5E8 antibody, a 6G5 antibody, a 2C8 antibody, a B3B11 antibody, and a 3G12 antibody.

In another aspect, the invention provides a tetravalent CD23 antibody molecule comprising four CD23 binding moieties and two heavy chain polypeptides, wherein two of said binding moieties are provided by an IgG antibody and two of said binding moieties are provided by two scFv molecules linked or fused to said IgG antibody.

In one embodiment, said IgG antibody comprises light chain (VL) and heavy chain (VH) variable domains derived from a 5E8 antibody. In one embodiment, said VL domain of said IgG antibody comprises the amino acid sequence of SEQ ID NO:97 and said VH domain of said IgG antibody comprises the amino acid sequence of SEQ ID NO:89.

In another embodiment, one or both of said scFv molecules comprise a light chain (VL) and a heavy chain (VH) variable domain derived from a 5E8 antibody. In one embodiment, said VL domain of said scFv molecules comprise the amino acid sequence of SEQ ID NO:97 and said VH domain of said scFv molecules comprise the amino acid sequence of SEQ ID NO:89.

In certain embodiments, one or both of said scFv molecules is a stabilized scFv molecule having a T50 of greater than 55° C. In one embodiment, one or both of said scFv molecules is a stabilized scFv molecule having a T50 that is at least 2° C.-10° C. higher than that of a conventional 5E8 scFv molecule (SEQ ID NO:6 or SEQ ID NO:8). In one embodiment, said stabilized scFv molecule is a stabilized scFv molecule comprising the amino acid sequence of pIEH252 (SEQ ID NO:10) or pIEH246 (SEQ ID NO:12).

In one embodiment, one or both of said scFv molecules are fused to said IgG antibody via a Gly/Ser linker. In one embodiment, said Gly/Ser linker is a (Gly₄Ser)₅ or Ser(Gly₄Ser)₃ linker. In one embodiment, said scFv molecules are linked or fused to said IgG antibody via the VL domain of said scFv molecules. In one embodiment, the scFv molecule is of the orientation VH→(Gly4Ser)ₙ linker→VL, and n is 3, 4, 5, or 6. In another embodiment, said scFv molecules are linked or fused to said IgG antibody via the VH domain of said scFv molecules. In one embodiment, the scFv molecule is of the orientation VL→(Gly4Ser)ₙ linker→VH, and n is 3, 4, 5 or 6.

In one embodiment, one or both of said scFv molecules is fused to a heavy chain of said IgG antibody to form one or both of the heavy chain polypeptides of said binding molecule. In one embodiment, one of said scFv molecules is linked or fused to a first heavy chain of said IgG antibody and one of said scFv molecules is linked or fused to a second heavy chain of said IgG antibody.

In one embodiment, one or both of said scFv molecules are linked or fused to the N-terminus of said first and second heavy chains of said IgG antibody. In one embodiment, the light chains of said IgG antibody comprise the light chain sequence of SEQ ID NO: 4; and the heavy chain polypeptides of said binding molecule comprise the amino acid sequence of SEQ ID NO:14 or SEQ ID NO:18. In one embodiment, said binding molecule is produced by the cell line 4F4 deposited on Sep. 26, 2008 as ATCC Deposit No. ______.

In one embodiment, one or both of said scFv molecules are fused to the C-terminus of said first and second heavy chains of said IgG antibody. In one embodiment, the light chains of said IgG antibody comprise the sequence of SEQ ID NO: 4 (p5E8) and the heavy chain polypeptides of said binding molecule comprise the amino acid sequence of SEQ ID NO:2, SEQ ID NO:16, or SEQ ID NO:20. In one embodiment, said binding molecule is produced by the cell line 1E2 deposited on Sep. 26, 2008 as ATCC Deposit No. ______.

In one embodiment, one or both of said scFv molecules is linked or fused to a light chain of said IgG antibody. In one embodiment, one of said scFv molecules is linked or fused to a first light chain of said IgG antibody and one of said scFv molecules is linked or fused to a second light chain of said IgG antibody. In one embodiment, one or both of said scFv molecules are linked or fused to the N-terminus of said first and second light chains of said IgG antibody.

In one embodiment, the IgG antibody comprises heavy chain constant domains of the human IgG4 isotype. In another embodiment, the IgG antibody comprises heavy chain constant domains of the human IgG1 isotype. In another embodiment, the heavy chain constant regions of said IgG antibody are afucosylated.

In one embodiment, the binding molecule of the invention is essentially resistant to aggregation when produced at commercial scale.

In another aspect, the invention provides a stabilized scFv molecule having binding specificity for CD23, wherein the stabilized scFv molecule has a T50 of greater than 55° C. In one embodiment, the stabilized scFv molecule comprises at least four stabilizing mutations as compared to a conventional scFv molecule, wherein said mutations are independently selected from the group consisting of:
a) substitution of an amino acid (e.g., glutamic acid) at Kabat position 6 of VH, e.g., with glutamine;
b) substitution of an amino acid (e.g., asparagine) at Kabat position 32 of VH, e.g., with serine;
c) substitution of an amino acid (e.g., serine) at Kabat position 49 of VH, e.g., with glycine or alanine;
d) substitution of an amino acid (e.g., proline) at Kabat position 56 of VH, e.g., with a histidine;
e) substitution of an amino acid (e.g., glutamic acid) at Kabat position 72 of VH, e.g., with aspartic acid;
f) substitution of an amino acid (e.g., valine) at Kabat position 50 of VL, e.g., with serine, aspartic acid, or glutamic acid;
g) substitution of an amino acid (e.g., valine) at Kabat position 75 of VL, e.g., with isoleucine; and
h) substitution of an amino acid (e.g., phenylalanine) at Kabat position 83 of VL, e.g., with serine, alanine, glycine, or threonine.

In certain embodiments, the scFv molecule is derived from a 5E8 antibody.

In one embodiment, the scFv molecule comprises at least one of the combinations of mutations selected from the group consisting of:
(i) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with glutamic acid, and (f) an amino acid (e.g., phenylalanine) at Kabat position 83 of VL, e.g., with alanine;
(ii) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with aspartic acid, and (f) an amino acid (e.g., phenylalanine) at Kabat position 83 of VL, e.g., with alanine;
(iii) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with glutamic acid, and (f) an amino acid (e.g., valine) at Kabat position 75 of VL, e.g., with isoleucine;
(iv) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with glutamic acid, and (e) an amino acid (e.g., phenylalanine) at Kabat position 83 of VL, e.g., with alanine;
(v) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with aspartic acid, and (e) an amino acid (e.g., phenylalanine) at Kabat position 83 of VL, e.g., with alanine;
(vi) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with serine, and (f) an amino acid (e.g., valine) at Kabat position 75 of VL, e.g., with isoleucine;
(vii) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with serine, and (e) an amino acid (e.g., phenylalanine) at Kabat position 83 of VL, e.g., with alanine;
(viii) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., proline) at Kabat position 56 of VH, e.g., with histidine; and (e) an amino acid (e.g., valine) at Kabat position 50 of VL, e.g., with aspartic acid;
(ix) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with serine, (f) an amino acid (e.g., valine) at Kabat position 75 of VL, e.g., with isoleucine, and (g) an amino acid (e.g., phenylalanine) at Kabat position 83 of VL, e.g., with alanine;
(x) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with aspartic acid, and (f) an amino acid (e.g., phenylalanine) at Kabat position 83 of VL, e.g., with alanine;
(xi) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with glutamic acid, and (f) an amino acid (e.g., phenylalanine) at Kabat position 83 of VL, e.g., with alanine;
(xii) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., glutamic acid) at Kabat position 72 of VH, e.g., with aspartic acid, (d) an amino acid (e.g., valine) at Kabat position 50 of VL, e.g., with glutamic acid, and (f) an amino acid (e.g., phenylalanine) at Kabat position 83 of VL, e.g., with alanine; and
(xiii) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with serine, and (f) an amino acid (e.g., phenylalanine) at Kabat position 83 of VL, e.g., with alanine.

In another aspect, the invention provides a binding molecule comprising at least three binding sites that bind to CD23, wherein the binding molecule comprises at least one stabilized scFv molecule having a T50 of greater than 55° C. In one embodiment, at least two of the binding sites are derived from an antibody selected from the group consisting of a 5E8 antibody, a 6G5 antibody, a 2C8 antibody, a B3B11 antibody, and a 3G12 antibody. In one embodiment, the binding molecule is an antibody molecule comprising at least one stabilized scFv molecule attached to its amino or carboxy terminus.

In another aspect the invention provides a tetravalent binding molecule that binds to CD23 and comprises two stabilized scFv molecules which have a T50 of greater than 55° C., wherein the binding molecule is resistant to aggregation when produced at commercial scale.

In another aspect, the invention provides a composition comprising a binding molecule of the invention and a carrier.

In another aspect, the invention provides a nucleic acid molecule encoding a binding molecule of the invention.

In another aspect, the invention provides a host cell comprising the nucleic acid molecule of the invention.

In another aspect, the invention provides a method of manufacturing a CD23 binding molecule comprising culturing the host cell of the invention under conditions such that the binding molecule is expressed, and isolating the binding molecule. In one embodiment, the host cell is cultured at commercial scale and at least 5 mg of the stabilized binding molecule is produced for every liter of the host cell culture medium. In another embodiment, the host cell is cultured at commercial scale and at least 50 mg of the stabilized binding molecule is produced for every liter of the host cell culture medium.

In another aspect, the invention provides a CD23 binding molecule manufactured according to said method. In one embodiment, the isolated binding molecule is resistant to aggregation when the host cell is cultured commercial scale.

In another aspect, the invention provides a method of decreasing tumor growth or metastasis in a human subject comprising administering to the subject an effective amount of a binding molecule. In another aspect, the invention provides a method of decreasing tumor growth or metastasis in a human subject comprising administering to the subject an effective amount of a binding molecule of the invention. In another embodiment, the method further comprises the administration of at least one additional agent (e.g., an anti-CD20 antibody (e.g., rituximab), fludarabine, and/or cyclophosphamide). In one embodiment, the human subject has chronic lymphocytic leukemia.

In another aspect, the invention provides a method of decreasing tumor growth or metastasis in a human subject comprising administering to the subject an effective amount of a tetravalent binding molecule that binds to CD23. In one embodiment, the human subject has chronic lymphocytic leukemia.

In another aspect, the invention provides a method of inducing CD23-mediated caspase-3 or PARP cleavage in a cancer cell bearing CD23, comprising contacting the cancer cell with a multivalent CD23 binding molecule comprising at least four CD23 binding moieties, wherein said binding molecule binds to FcγR and specifically crosslinks at least two distinct human CD23 molecules on the surface of the cancer cell. In one embodiment, the cancer cell is a CLL cell.

In one embodiment, said cleavage is induced to a greater extent than an equimolar amount of an antibody dimer formed by crosslinking of two bivalent CD23 monoclonal antibodies with a cross-linker. In one embodiment, the binding molecule induces cleavage 1.5 fold or more, 2-fold or more, 3-fold or more, 4-fold or more, 5-fold or more, 6 fold or more, 7-fold or more, 8 fold or more, 9-fold or more, 10-fold or more, and 15-fold or more than the equimolar amount of antibody dimer. In another embodiment, the equimolar amount is an amount selected from the group consisting of 1 ug/ml or more, 2 ug/ml or more, 5 ug/ml or more, 10 ug/ml or more, 15 ug/ml or more, and 20 ug/ml or more.

In one embodiment, three, four, five, six, seven, eight, nine, ten, or more CD23 molecules are crosslinked by said multivalent CD23 binding molecule. In one embodiment, the cross-linker is an antibody which binds to the anti-CD23 antibody. In one embodiment, the cross-linker is an engineered disulfide bond. In one embodiment, said binding molecule is a tetravalent CD23 antibody molecule comprising four CD23 binding moieties and two heavy chain polypeptides, wherein two of said binding moieties are provided by an IgG antibody and two of said binding moieties are provided by two scFv molecules linked or fused to said IgG antibody.

### BRIEF DESCRIPTION OF THE DRAWINGS

FIG. 1 depicts binding of exemplary tetravalent CD23 binding molecules of the invention to CD23 receptors on CLL tumor cell and formation of CD23 receptor complexes by cross-linking of individual CD23 receptors. CD23 cross-linking results in enhanced tumor cell death due to induction of apoptosis. Cell death is further enhanced in relation to the number of CD23 molecules that are cross-linked. In one embodiment, two CD23 molecules are cross-linked. In another embodiment, three or four CD23 molecules are cross-linked. In another embodiment, five CD23 molecules are cross-linked.

FIG. 2 depicts exemplary CD23 multivalent binding molecules of the invention. The anti-CD23 multivalent binding molecules are formed by the fusion or linkage of anti-CD23 (p5E8) scFv molecules of the invention to a p5E8 IgG antibody. In preferred embodiments, at least one (more preferably all) of the scFv molecules is a stabilized scFv molecule. In other embodiments, all of the scFv molecules are conventional scFv molecules. In certain embodiments, scFv molecules may be fused or linked to either the C-terminus or N-terminus of the heavy chain or to the N-terminus of the antibody light chain (see, e.g., FIG. 2A). In one embodiment, scFv molecules may also be directly fused or linked to both (i) the N-terminus of CH1 domain of an antibody heavy chain further comprising an Fc domain or portion thereof and (ii) the N-terminus of the light chain CL domain as exemplified by an (scFv)₄-Fc format (FIG. 2B). In another embodiment, scFv molecules are fused or linked in series to the N-terminus of an Fc domain or portion thereof as exemplified by the single chain scFv₂-Fc format (FIG. 2C).

FIG. 3 shows the single-stranded DNA sequence (SEQ ID NO:1, FIG. 3A) and amino acid sequence (SEQ ID NO:2, FIG. 3B) of heavy chain C-terminal tetravalent p5E8 antibody comprising a conventional scFv linked to the C-terminus of a p5E8.

FIG. 4 shows the single-stranded DNA sequence (SEQ ID NO:3, FIG. 4A) and amino acid sequence (SEQ ID NO: 4; FIG. 4B) of the p5E8 light chain.

FIG. 5 shows the single-stranded DNA sequence (SEQ ID NO:5, FIG. 5A) and amino acid sequence (SEQ ID NO: 6; FIG. 5B) of a conventional p5E8 (VL/VH) scFv.

FIG. 6 shows the single-stranded DNA sequence (SEQ ID NO:7, FIG. 6A) and amino acid sequence (SEQ ID NO: 8; FIG. 6B) of a conventional p5E8 (VH/VL) scFv.

FIG. 7 depicts the results of a thermal challenge assay in which the thermal stabilities of the conventional p5E8 (VH/VL) scFv (solid line) and p5E8 (VL/VH) scFv (dashed line) molecules are compared. The temperatures at which 50% of the scFv molecules retain their binding activity (T₅₀) are indicated in the figure.

FIG. 8 depicts the single-stranded DNA sequence (SEQ ID NO:9; FIG. 8A) and amino acid sequence (SEQ ID NO: 10; FIG. 8B) of stabilized p5E8 pIEH252 scFv.

FIG. 9 depicts the single-stranded DNA sequence (SEQ ID NO:11; FIG. 9A) and amino acid sequence (SEQ ID NO: 12; FIG. 9B) of stabilized p5E8 pIEH246 scFv.

FIG. 10 depicts the single-stranded DNA sequence (SEQ ID NO:13; FIG. 10A) and amino acid sequence (SEQ ID NO: 14; FIG. 10B) of heavy chain N-terminal tetravalent p5E8 comprising the stabilized pIEH252 scFv.

FIG. 11 depicts the single-stranded DNA sequence (SEQ ID NO:15; FIG. 11A) and amino acid sequence (SEQ ID NO: 16; FIG. 11B) of heavy chain C-terminal tetravalent p5E8 comprising the stabilized pIEH252 scFv.

FIG. 12 depicts the single-stranded DNA sequence (SEQ ID NO:17; FIG. 12A) and amino acid sequence (SEQ ID NO: 18; FIG. 12B) of heavy chain N-terminal tetravalent p5E8 comprising the stabilized pIEH246 scFv.

FIG. 13 depicts the single-stranded DNA sequence (SEQ ID NO:19; FIG. 13A) and amino acid sequence (SEQ ID NO: 20; FIG. 13B) of heavy chain C-terminal tetravalent p5E8 comprising the stabilized pIEH246 scFv.

FIG. 14 depicts a schematic diagram of an exemplary stabilized two chain dimeric tetravalent CD23 minibody (stabilized Bispecific N-scFv tetravalent CD23 minibody) comprising stabilized scFv fragments of the invention appended to the amino termini. The exemplary stabilized bivalent tetravalent minibody comprises 4 stabilized scFvs with binding specificity for CD23 antigen. However, in other embodiments, at least one of the stabilized scFvs may have specificity for a different CLL antigen (e.g., CD20). For example, the tetravalent minibody can also be constructed such that each chain portion contains 2 stabilized scFv fragments with different binding specificities (e.g., a CD23 specificity and a CD20 specificity). In another embodiment, the orientation of the VH and VL domains in the scFv may be changed. In another embodiment, fewer than all of the scFvs are stabilized.

FIG. 15 depicts a schematic diagram of an exemplary stabilized two chain dimeric tetravalent CD23 minibody (stabilized C-scFv tetravalent CD23 minibody) comprising stabilized CD23 scFv fragments appended to both carboxyl termini of a bivalent CD23 minibody. The exemplary stabilized bivalent tetravalent minibody comprises 4 stabilized scFvs with binding specificity for CD23 antigen. However, in other embodiments, at least one of the stabilized scFvs has binding specificity for a different CLL antigen (e.g., CD20). For example, the tetravalent minibody can also be constructed such that each chain portion contains 2 stabilized scFv fragments with different specificities (e.g., a CD23 specificity and a CD20 specificity). In another embodiment, the orientation of the VH and VL domains in the scFv may be changed. In another embodiment, fewer than all of the scFvs are stabilized.

FIG. 16 shows a schematic diagram of a stabilized four chain dimeric CD23 diabody comprising stabilized CD23 scFvs of the invention. However, in other embodiments, at least one of the stabilized scFvs has binding specificity for a different CLL antigen (e.g., CD20). For example, the diabody can also be constructed such that each arm contains stabilized scFv fragments with different specificities (e.g., a CD23 specificity and a CD20 specificity). In another embodiment, the orientation of the VH and VL domains in the scFv may be changed. In another embodiment, fewer than all of the scFvs are stabilized. In yet another embodiment, the diabody comprises a full length Fc region.

FIG. 17 shows a schematic diagram of a stabilized four-chain tetravalent scFv CH2 domain deleted CD23 antibody (stabilized C-scFv tetravalent CH2 domain deleted CD23 antibody) comprising a stabilized CD23 scFv appended to the carboxyl terminus of CH3 and a hinge connecting peptide. Each heavy chain portion of the antibody contains a Fv region and a stabilized scFv region with binding specificity for the CD23 antigen. However, in other embodiments, at least one of the Fv or scFv regions has binding specificity for a different CLL antigen (e.g., CD20). For example, the domain deleted antibody can also be constructed such that each arm contains stabilized scFv fragments with different specificities (e.g., a CD23 specificity and a CD20 specificity). The orientation of the VH and VL domains in the stabilized scFv may be changed and the respective antigen binding specificities may be altered. In another embodiment, fewer than all of the scFvs are stabilized.

FIG. 18 shows a schematic diagram of a stabilized four-chain tetravalent scFv CH2 domain deleted CD23 antibody (stabilized N_{H}-scFv tetravalent CH2 domain deleted CD23 antibody) comprising a stabilized CD23 scFv appended to the amino terminus of VH and comprising a hinge connecting peptide. Each heavy chain portion of the bispecific antibody contains an Fv region and a stabilized scFv region with binding specificity for CD23 antigen. However, in other embodiments, at least one of the Fv or scFv regions has binding specificity for a different CLL antigen (e.g., CD20). For example, the domain-deleted antibody can also be constructed such that each arm contains stabilized scFv fragments with different specificities (e.g., a CD23 specificity and a CD20 specificity). The orientation of the VH and VL domains in the stabilized scFv may be changed and the respective antigen binding specificities may be altered. In another embodiment, fewer than all of the scFvs are stabilized.

FIG. 19 shows a schematic diagram of a stabilized four-chain tetravalent scFv CH2 domain deleted bispecific antibody (stabilized N_{L}-scFv tetravalent CH2 domain deleted bispecific antibody) comprising a stabilized scFv appended to the amino terminus of VL and comprising a hinge connecting peptide. Each heavy chain portion of the bispecific antibody contains an Fv region and a stabilized scFv region with binding specificity for the CD23 antigen. The orientation of the VH and VL domains in the stabilized scFv may be changed and the respective antigen binding specificities may be altered. In another embodiment, fewer than all of the scFvs are stabilized.

FIG. 20 depicts summary data of biochemical and biophysical properties of exemplary stability engineered p5E8 scFv molecules.

FIG. 21A shows an SDS-PAGE gel of purified stability-engineered C-Tetravalent p5E8 (pXWU103). FIG. 21B shows an analytical SEC elution profile of purified stability-engineered C-Tetravalent p5E8 (pXWU103).

FIG. 22 A shows an SDS-PAGE gel of purified stability-engineered N-Tetravalent p5E8 (pXWU104). FIG. 22 B shows an analytical SEC elution profile of purified stability-engineered N-Tetravalent p5E8 (pXWU104).

FIG. 23. Stoichiometry of CD23 binding to C-terminal tetravalent p5E8, p5E8 IgG and p5E8 Fab as measured by solution phase competition Biacore. (A) Representative sensorgrams showing binding of CD23 to a p5E8 chip surface by unbound CD23 in a CD23/C-terminal tetravalent p5E8 equilibrium at increasing antibody concentration. (B) Plot of initial binding rates (Vᵢ) vs. concentration of antibody. Derived N values are summarized in Table 7. No antibody (), circles; C-terminal tetravalent p5E8 (▴), triangles; p5E8 IgG(▪), squares; and p5E8 Fab (♦), diamonds.

FIG. 24. (A) Thermograms recorded as a function of CD23 (ligand) added to p5E8 Fab that have been integrated with respect to time and normalized per mole of added ligand. (B) The fitted binding isotherms plotted as a function of molar ligand/antibody ratio.

FIG. 25. Binding of N-terminal and C-terminal tetravalent p5E8 to human FcγRIIIa (V158) measured by competition AlphaScreen (n=2). Agly IgG4 control antibody [symbol] : p5E8 IgG (); N-terminal tetravalent p5E8 (▪); and C-terminal tetravalent p5E8 (▴). IC₅₀ values are as follows: p5E8 IgG, 0.63 μM; N-terminal tetravalent p5E8, 0.49 μM; and C-terminal tetravalent p5E8, 0.23 μM.

FIG. 26 depicts antigen binding of tetravalent p5E8. CHO cells expressing human CD23 were incubated in different concentrations of unconjugated antibodies at 4° C. for 1 hour. Cells were washed, incubated in PE-conjugated secondary antibody for 30 mins at 4° C. prior to washing and analysis by flow cytometry.

FIG. 27 depicts antigen binding of tetravalent p5E8. (A) SKW6.4 CD23+ lymphoma and (B) MEC1 CD23+ CLL human cell lines were incubated in different concentrations of unconjugated antibodies at 4° C. for 1 hour. Cells were washed, incubated in PE-conjugated secondary antibody for 30 mins at 4° C. prior to washing and analysis by flow cytometry.

FIG. 28 depicts ADCC activity of tetravalent p5E8. (A) SKW6.4 cells or (B) primary CLL patient B cells were incubated in increasing concentrations of antibody in the presence of activated human PBMC. The ADCC activity of antibodies was determined by measuring 51Cr release after a 4 hour incubation as a readout of cell lysis. Results shown are representative of 3 independent experiments (SKW6.4 cells) or as the mean of 4 patient samples (CLL) with standard deviations.

FIG. 29 depicts the CDC activity of tetravalent p5E8. (A) SKW6.4 cells or (B) primary CLL patient B cells were incubated in increasing concentrations of antibody in the presence of human serum complement. The CDC activity of antibodies was determined by 2 hours later measuring cell viability using a Cell Titer Glo assay. Results shown are representative of 3 independent experiments (SKW6.4 cells) or as the mean of 4 patient samples (CLL) with standard deviations.

FIG. 30 depicts the apoptotic activity of tetravalent p5E8. (A) SKW6.4 and (B) MEC1 cells were incubated with 10 μg/ml antibodies followed by incubation with a secondary antibody to mediate cross-linking. Apoptosis was scored 48 hours later by annexin V staining and flow cytometric analysis. Staurosporine was used as a positive control for apoptosis. Results shown are representative of 3 independent experiments with standard deviations.

FIG. 31 depicts the ability of tetravalent p5E8 to induce apoptosis in the presence or absence of exogenous cross-linking. SKW6.4 cells were incubated with increasing concentrations of antibodies followed by incubation with (A) a secondary antibody to mediate cross-linking or (B) no treatment. Apoptosis was scored 48 hours later by annexin V staining and flow cytometric analysis. Results shown are representative of 3 independent experiments with standard deviations.

FIG. 32 depicts the apoptotic signaling induced by tetravalent p5E8. SKW6.4 cells were cultured in the presence of increasing concentrations of antibody in the presence (x-) or absence of exogenous cross-linking secondary antibody. Protein lysates were prepared and an ELISA was performed to measure the activation of (A) PARP and (B) caspase 3.

FIG. 33 depicts the apoptotic signaling induced by tetravalent p5E8. SKW6.4 cells were cultured in the presence of 10 μg/ml of antibody in the presence (x-) or absence of exogenous cross-linking secondary antibody. Protein lysates were run on SDS-PAGE gels, transferred to nitrocellulose and probed with antibodies against PARP, cleaved caspase 3, full-length caspase 3, XIAP. GAPDH was used to ensure even loading.

FIG. 34 depicts DNA fragmentation induced by tetravalent p5E8. SKW6.4 cells were incubated with increasing concentrations of antibodies antibody in the presence (x-) or absence of exogenous cross-linking secondary antibody. DNA fragmentation was scored 48 hours later by Apo-BrdU staining and flow cytometric analysis. Results shown are representative of 3 independent experiments with standard deviations.

FIG. 35 depicts the apoptotic activity of tetravalent p5E8 in CLL B cells cultured ex vivo. CLL B cells from one patient were incubated with 10 μg/ml of p5E8 antibodies alone or in combination with alemtuzumab. Cells were subsequently incubated with a secondary antibody to mediate cross-linking. Apoptosis was scored 48 hours later by annexin V staining and flow cytometric analysis. Results shown are representative of 3 independent experiments and displayed as the mean of triplicate samples with standard deviations.

FIG. 36 depicts the plasma concentration-time curve following intraperitoneal administration of a 10 mg/kg bolus of tetravalent p5E8 antibody.

### DETAILED DESCRIPTION OF THE INVENTION

The invention is based, at least in part, on the development of CD23 binding molecules with improved anti-tumor potency and/or protein stability.

In certain aspects the invention provides multivalent CD23 binding molecules which are capable of cross-linking two or more CD23 molecules on the surface of an immune cell (e.g., a neoplastic B cell such as a CLL cell). Preferably, the multivalent binding molecules are capable of cross-linking said CD23 molecules in the absence of a cross-linker. The biological consequence of contacting a multivalent CD23 binding molecule with a CD23 target molecule expressed on an immune cell (e.g., a CLL cell) is an increased level of receptor cross-linking and more efficacious induction of apoptosis/cellular death. This improvement in potency is expected to translate into clinical benefit by providing a more potent and efficacious therapeutic agent. Crosslinking of these multivalent binding molecules (e.g., by FcγR binding or the use of an crosslinker, e.g., a secondary antibody) may further increase their potency.

In certain embodiments, the multivalent CD23 binding molecules of the invention are CD23 antibody molecules comprising more than two CD23 binding moieties and two heavy chain polypeptides. In one embodiment, at least two of said binding moieties are provided by an IgG antibody and at least one (preferably two) of said binding moieties are provided by scFv molecules linked or fused (e.g., recombinantly fused) to said IgG antibody. Such CD23 binding molecules provide additional advantages in that more than 50% (and preferably all) of the molecules are in multivalent form following expression in a host cell.

In other aspects, the invention provides stabilized scFv molecules with CD23 binding specificity. The stabilized scFv molecules of the instant invention are especially useful in producing stable multivalent CD23 molecules. The stabilized CD23 binding molecules of the invention can be stably expressed in culture, are suitable for large scale production, are resistant to aggregation and are stable in vivo.

The invention is also based, at least in part, on the development of stabilized CD23 binding molecules that consist of or comprise a stabilized CD23 scFv molecule and methods for making such stabilized CD23 binding molecules.

Before further description of the invention, for convenience, certain terms are described below:

### I. DEFINITIONS

As used herein, the term “binding molecule” refers to a molecule which binds (e.g., specifically binds or preferentially binds) to a target molecule of interest, e.g., an antigen. In particular embodiments, a binding molecule of the invention is a polypeptide comprising a binding site which specifically or preferentially binds to CD23.

As used herein the term “scFv molecule” includes binding molecules which consist of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. scFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

A “scFv linker” as used herein refers to a moiety interposed between the VL and VH domains of the scFv. scFv linkers preferably maintain the scFv molecule in a antigen binding conformation. In one embodiment, an scFv linker comprises or consists of an scFv linker peptide. In certain embodiments, an scFv linker peptide comprises or consists of a gly-ser connecting peptide. In other embodiments, an scFv linker comprises a disulfide bond.

As used herein, the term “gly-ser connecting peptide” refers to a peptide that consists of glycine and serine residues. An exemplary gly/ser connecting peptide comprises the amino acid sequence (Gly₄ Ser)ₙ In one embodiment, n=1. In one embodiment, n=2. In another embodiment, n=3. In a preferred embodiment, n=4, i.e., (Gly₄ Ser)₄. In another embodiment, n=5. In yet another embodiment, n=6. Another exemplary gly/ser connecting peptide comprises the amino acid sequence Ser(Gly₄Ser)ₙ. In one embodiment, n=1. In one embodiment, n=2. In a preferred embodiment, n=3. In another embodiment, n=4. In another embodiment, n=5. In yet another embodiment, n=6.

As used herein the term “disulfide bond” refers to the covalent bond formed between two sulfur atoms. The amino acid cysteine comprises a thiol group that can form a disulfide bond or bridge with a second thiol group. In certain embodiments, the disulfide bond is engineered.

As used herein the term “conventional scFv molecule” refers to an scFv molecules which is not a stabilized scFv molecule. For example, a typical conventional scFv lacks stabilizing mutations and comprises a VH and a VL domain linked by a (G₄S)3 linker.

A “stabilized scFv molecule” of the invention is an scFv molecule comprising at least one change or alteration as compared to a conventional scFv molecule which results in stabilization of the scFv molecule. As used herein, the term “stabilizing mutation” includes a mutation which confers enhanced protein stability (e.g. thermal stability) to the scFv molecule and/or to a larger protein comprising said scFv molecule. In one embodiment, the stabilizing mutation comprises the substitution of a destabilizing amino acid with a replacement amino acid that confers enhanced protein stability (herein a “stabilizing amino acid”). In one embodiment, the stabilizing mutation is one in which the length of an scFv linker has been optimized. In one embodiment, a stabilized scFv molecule of the invention comprises one or more amino acid substitutions. For example, in one embodiment, a stabilizing mutation comprises a substitution of at least one amino acid residue which substitution results in an increase in stability of the VH and VL interface of an scFv molecule. In one embodiment, the amino acid is within the interface. In another embodiment, the amino acid is one which scaffolds the interaction between VH and VL. In another embodiment, a stabilizing mutation comprises substituting at least one amino acid in the VH domain or VL domain that covaries with two or more amino acids at the interface between the VH and VL domains. In another embodiment, the stabilizing mutation is one in which at least one cysteine residue is introduced (i.e., is engineered into one or more of the VH or VL domain) such that the VH and VL domains are linked by at least one disulfide bond between an amino acid in the VH and an amino acid in the VL domain. In certain preferred embodiments, a stabilized scFv molecule of the invention is one in which both the length of the scFv linker is optimized and at least one amino acid residue is substituted and/or the VH and VL domains are linked by a disulfide bond between an amino acid in the VH and an amino acid in the VL domain. In one embodiment, more than one of the stabilizing mutations described herein may be made in a scFv molecule.

In one embodiment, one or more stabilizing mutations made to an scFv molecule simultaneously improve the thermal stability of both the VH and VL domains of the scFv molecule as compared to a conventional scFv molecule.

Preferably, a population of one or more of the stabilized scFv molecules of the invention is expressed as a population of monomeric, soluble proteins. In one embodiment, no more than 10% (e.g., 10%, 5%, 2% or 1%) is present in aggregated form. In one embodiment, the stabilized scFv molecules of the population may comprise the same stabilizing mutation or a combination of stabilizing mutations. In other embodiments, the individual stabilized scFv molecules of the population comprise different stabilizing mutations.

The subject stabilized scFv molecules may be used alone to bind to a target molecule or may be linked to another polypeptide to form stabilized binding molecules which comprise a stabilized scFv molecule. For example, a binding molecule of the invention may comprise an scFv molecule linked to a second scFv molecule or a non-scFv molecule, e.g., that imparts target binding specificity, such as an antibody.

As used herein the term “protein stability” refers to an art-recognized measure of the maintenance of one or more physical properties of a protein in response to an environmental condition (e.g. an elevated or lowered temperature). In one embodiment, the physical property is the maintenance of the covalent structure of the protein (e.g. the absence of proteolytic cleavage, unwanted oxidation or deamidation). In another embodiment, the physical property is the presence of the protein in a properly folded state (e.g. the absence of soluble or insoluble aggregates or precipitates). In one embodiment, stability of a protein is measured by assaying a biophysical property of the protein, for example thermal stability, pH unfolding profile, stable removal of glycosylation, solubility, biochemical function (e.g., ability to bind to a protein (e.g., a ligand, a receptor, an antigen, etc.) or chemical moiety, etc.), and/or combinations thereof. In another embodiment, biochemical function is demonstrated by the binding affinity of an interaction. In one embodiment, a measure of protein stability is thermal stability, i.e., resistance to thermal challenge. Stability can be measured using methods known in the art and/or described herein.

The VL and VH domains of an scFv molecule are derived from one or more antibody molecules. It will also be understood by one of ordinary skill in the art that the variable regions of the scFv molecules of the invention may be modified such that they vary in amino acid sequence from the antibody molecule from which they were derived. For example, in one embodiment, nucleotide or amino acid substitutions leading to conservative substitutions or changes at amino acid residues may be made (e.g., in CDR and/or framework residues). Alternatively or in addition, mutations may be made to CDR amino acid residues to optimize antigen binding using art recognized techniques. The binding molecules of the invention maintain the ability to bind to antigen.

As used herein the term “derived from” a designated protein refers to the origin of the polypeptide. In one embodiment, the polypeptide or amino acid sequence which is derived from a particular starting polypeptide is a variable region sequence (e.g. a VH or VL) or sequence related thereto (e.g. a CDR or framework region). In one embodiment, the amino acid sequence which is derived from a particular starting polypeptide is not contiguous. For example, in one embodiment, one, two, three, four, five, or six CDRs are derived from a starting antibody. In one embodiment, the polypeptide or amino acid sequence that is derived from a particular starting polypeptide or amino acid sequence has an amino acid sequence that is essentially identical to that of the starting sequence or a portion thereof, wherein the portion consists of at least 3-5 amino acids, 5-10 amino acids, at least 10-20 amino acids, at least 20-30 amino acids, or at least 30-50 amino acids, or which is otherwise identifiable to one of ordinary skill in the art as having its origin in the starting sequence.

An isolated nucleic acid molecule encoding a stabilized scFv molecule or a portion thereof can be created by introducing one or more nucleotide substitutions, additions or deletions into the nucleotide sequence of a conventional scFv molecule or an immunoglobulin from which it is derived such that one or more amino acid substitutions, additions or deletions are introduced into the encoded protein. Mutations may be introduced by standard techniques, such as site-directed mutagenesis and PCR-mediated mutagenesis. In one embodiment, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue in an immunoglobulin polypeptide may be replaced with another amino acid residue from the same side chain family. In another embodiment, a string of amino acids can be replaced with a structurally similar string that differs in order and/or composition of side chain family members. In another embodiment, a mutation is introduced in order to introduce at least one cysteine molecule into the VH and into the VL domain and, thereby, introduce a disulfide bond into the scFv molecule. In another embodiment, an amino acid of a conventional scFv molecule may be substituted with an amino acid having similar physical (e.g., spatial) or functional properties. Preferably, amino acids substituted into conventional scFv molecules are compatible with the integrity of the V_{L}/V_{H} interface, CDR conformations, and V_{H} and/or V_{L} folding.

Alternatively, in another embodiment, mutations may be introduced randomly along all or part of the immunoglobulin coding sequence.

The stabilized scFv molecules of the invention or polypeptides comprising the stabilized scFv molecules are binding molecules, i.e., they bind to a target molecule of interest, e.g., an antigen. When a stabilized scFv molecule of the invention is fused to a second molecule, the second molecule may also impart a binding specificity to the fusion protein.

In one embodiment, the binding molecules of the invention are monovalent, i.e., comprise one target binding site (e.g., as in the case of an scFv molecule). In one embodiment, the binding molecules of the invention are multivalent, i.e., comprise more than one target binding site. In another embodiment, the binding molecules comprise at least two binding sites. In one embodiment, the binding molecules comprise two binding sites. In one embodiment, the binding molecules comprise three binding sites. In another embodiment, the binding molecules comprise four binding sites. In another embodiment, the binding molecules comprise greater than four binding sites.

In one embodiment, the binding molecules of the invention are monomers. In another embodiment, the binding molecules of the invention are multimers. For example, in one embodiment, the binding molecules of the invention are dimers. In one embodiment, the dimers of the invention are homodimers, comprising two identical monomeric subunits. In another embodiment, the dimers of the invention are heterodimers, comprising two non-identical monomeric subunits. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

As used herein the term “valency” refers to the number of potential target binding sites in a polypeptide. Each target binding site specifically binds one target molecule or specific site on a target molecule. When a polypeptide comprises more than one target binding site, each target binding site may specifically bind the same or different molecules (e.g., may bind to different molecules, e.g., different antigens, or different epitopes on the same molecule).

As used herein, the term “multivalent” refers to a binding molecule having more than one binding site. Preferred multivalent binding molecules have more than two binding sites and the term “hypervalent” may be used to describe such molecules. For example, the binding molecules of the invention may be bivalent (two binding sites), trivalent (three binding sites), tetravalent (four binding sites), pentavalent (five binding sites), hexavalent (six binding sites), heptavalent (seven binding sites), or of higher order valency (e.g., octavalent (eight binding sites) or decavalent (ten binding sites)).

As used herein, the term “cross-linker” refers to an agent that facilitates the multimerization, dimerization or cross-linking of two or more binding molecules (e.g., two or more antibodies). Exemplary cross-linkers include engineered disulfide bonds, chemical cross-linkers (heterobifunctional linkers) or secondary antibodies.

The term “specificity” refers to the ability to specifically bind (e.g., immunoreact with) a given target. A polypeptide may be monospecific and contain one or more binding sites which specifically bind a target (e.g., CD23) or a polypeptide may be multispecific (e.g., bispecific or trispecific) and contain two or more binding sites which specifically bind the same target (e.g., CD23) or different targets (e.g., CD23 and another target). Specific binding may be imparted by a stabilized scFv molecule of the invention and/or a non-scFv moiety to which a stabilized scFv molecule of the invention is linked.

In one embodiment, a binding molecule of the invention is multispecific. For example, in one embodiment, a multi specific binding molecule of the invention is a bispecific molecule having binding specificity for at least two targets, e.g., more than one target molecule or more than one epitope on the same target molecule. In one embodiment, a multispecific molecule has at least one binding site specific for a molecule targeted for reduction or elimination and a target molecule on a cell. In another embodiment, a multispecific molecule has at least one target binding site specific for a molecule targeted for reduction or elimination and at least one binding site specific for a drug. In yet another embodiment, a multispecific molecule has at least one binding site specific for a molecule targeted for reduction or elimination and at least one binding site specific for a prodrug.

In one embodiment, a multispecific molecule comprises one specificity for a soluble molecule and one specificity for a cell surface molecule (e.g., CD23). In another embodiment, a multispecific molecule has two binding specificities for two targets present on one or more soluble molecules. In another embodiment, a multispecific molecule has two binding specificities for two targets present on one or more cell surface molecules (which may be present on one or more cells).

In one embodiment, the binding molecules have at least one target binding site specific for a molecule which mediates a biological effect (e.g., which modulates cellular activation (e.g., by binding to a cell surface receptor and resulting in transmission or inhibition of an activating or inhibitory signal), which results in death of the cell (e.g., by a cell signal induced pathway, by complement fixation or exposure to a payload present on the binding molecule), or which modulates a disease or disorder in a subject (e.g., by mediating or promoting cell killing, by promoting lysis of a fibrin clot or promoting clot formation, or by modulating the amount of a substance which is bioavailable (e.g., by enhancing or reducing the amount of a ligand such as TNFα in the subject)).

In another embodiment, the binding molecules of the invention bind at least one target that transduces a signal to a cell, e.g., by binding to a cell surface receptor, such CD23. By “transduces a signal” it is meant that by binding to the cell, the binding molecule converts the extracellular influence on the cell surface receptor into a cellular response, e.g., by modulating a signal transduction pathway.

In one embodiment, the binding molecules bind at least one target binding site specific for a molecule targeted for reduction or elimination, e.g., a cell surface antigen or a soluble antigen. In one embodiment, the binding of the binding molecule to the target results in reduction or elimination of the target, e.g., from a tissue or from the circulation. In another embodiment, the binding molecules have at least one binding site specific for a molecule that can be used to detect the presence of a target molecule (e.g., to detect a contaminant or diagnose a condition or disorder). In yet another embodiment, a binding molecule of the invention comprises at least one binding site that targets the binding molecule to a specific site in a subject (e.g., to a tumor cell or blood clot).

In a preferred embodiment, a multispecific molecule is a tetravalent antibody that has four binding sites. A tetravalent molecule may be bispecific and bivalent for each specificity. Further description of exemplary bispecific molecules is provided below.

Preferred binding molecules of the invention comprise framework and constant region amino acid sequences derived from a human amino acid sequence. However, binding polypeptides may comprise framework and/or constant region sequences derived from another mammalian species. For example, binding molecules comprising murine sequences may be appropriate for certain applications. In one embodiment, a primate framework region (e.g., non-human primate), heavy chain portion, and/or hinge portion may be included in the subject binding molecules. In one embodiment, one or more murine amino acids may be present in the framework region of a binding polypeptide, e.g., a human or non-human primate framework amino acid sequence may comprise one or more amino acid back mutations in which the corresponding murine amino acid residue is present and/or may comprise one or mutations to a different amino acid residue not found in the starting murine antibody. Preferred binding molecules of the invention are less immunogenic than murine antibodies.

A “fusion” or chimeric protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A fusion protein may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a genotypically distinct entity from that of the entity to which it is being compared. For instance, a heterologous polynucleotide or antigen may be derived from a different species, different cell type of an individual, or the same or different type of cell of distinct individuals.

The term “ligand binding domain” or “ligand binding portion” as used herein refers to any native receptor (e.g., cell surface receptor) or any region or derivative thereof retaining at least a qualitative ligand binding ability, and preferably the biological activity of a corresponding native receptor.

The term “receptor binding domain” or “receptor binding portion” as used herein refers to any native ligand or any region or derivative thereof retaining at least a qualitative receptor binding ability, and preferably the biological activity of a corresponding native ligand.

In one embodiment, the binding molecules of the invention are stabilized “antibody” or “immunoglobulin” molecules, e.g., naturally occurring antibody or immunoglobulin molecules (or an antigen binding fragment thereof) or genetically engineered antibody molecules that bind antigen in a manner similar to antibody molecules and that comprise an scFv molecule of the invention. As used herein, the term “immunoglobulin” includes a polypeptide having a combination of two heavy and two light chains whether or not it possesses any relevant specific immunoreactivity. “Antibodies” refers to such assemblies which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “immunoglobulin” comprises five distinct classes of that can be distinguished biochemically. All five classes of antibodies are within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

Stabilizing mutations to scFv molecules may be made to amino acids in the CDR and/or in the framework regions of an scFv variable heavy and/or variable light chains. As used herein the term “variable region CDR amino acid residues” includes amino acids in a CDR or complementarity determining region as identified using sequence or structure based methods. As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein the term “variable region framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

As previously indicated, the subunit structures and three dimensional configuration of the constant regions of the various immunoglobulin classes are well known. As used herein, the term “VH domain” includes the amino terminal variable domain of an immunoglobulin heavy chain. As used herein, the term “VL domain” includes the amino terminal variable domain of an immunoglobulin light chain.

The term “fragment” refers to a part or portion of a polypeptide (e.g., an antibody or an antibody chain) comprising fewer amino acid residues than an intact or complete polypeptide. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As used herein, the term “fragment” of an antibody molecule includes antigen-binding fragments of antibodies, for example, an antibody light chain (VL), an antibody heavy chain (VH), a single chain antibody (scFv), a F(ab′)2 fragment, a Fab fragment, an Fd fragment, an Fv fragment, and a single domain antibody fragment (DAb). Fragments can be obtained, e.g., via chemical or enzymatic treatment of an intact or complete antibody or antibody chain or by recombinant means.

As used herein, the term “binding moiety”, “binding site”, or “binding domain” refers to the portion of a binding molecule that is responsible for selectively binding to a target molecule of interest (e.g. an antigen, ligand, receptor, substrate or inhibitor). Exemplary binding domains include an antibody variable domain (e.g., a VL or VH domain), a receptor binding domain of a ligand, a ligand binding domain of a receptor or an enzymatic domain. In preferred embodiments, the binding molecules have binding moieties specific for a target CD23 molecule, e.g., a human CD23 molecule. In certain embodiments, the binding moieties have a single CD23 binding specificity. In other embodiments, the binding moieties may have two or more binding specificities (e.g., wherein at least one binding specificity is a CD23 binding specificity).

Binding molecules of the invention can be made using techniques that are known in the art. In one embodiment, the polypeptides of the invention are “recombinantly produced,” i.e., are produced using recombinant DNA technology. Exemplary techniques for making such molecules are discussed in more detail below.

In one embodiment, a binding molecule of the invention is a naturally occurring antibody to which a stabilized scFv molecule has been fused. In one embodiment, a binding molecule of the invention is a modified antibody to which a stabilized scFv molecule has been fused. As used herein, the term “modified antibody” includes synthetic forms of antibodies which are altered such that they are not naturally occurring. In another embodiment, a binding molecule of the invention is a fusion protein comprising at least one scFv molecule.

In preferred embodiments, a polypeptide of the invention will not elicit a deleterious immune response in a human.

In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Exemplary modifications include additions, deletions or substitutions of one or more amino acids in one or more domains. Such changes may be included to optimize effector function, half-life, etc.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques). Preferably, the binding molecules of the invention are made using such methods.

As used herein, the terms “linked,” “fused” or “fusion” are used interchangeably. These terms refer to the joining together of two more elements or components, by whatever means including chemical conjugation or recombinant means. Preferably the polypeptides which are fused are genetically fused, i.e., are fused using recombinant DNA technology. An “in-frame fusion” refers to the joining of two or more open reading frames (ORFs) to form a continuous longer ORF, in a manner that maintains the correct reading frame of the original ORFs. Thus, the resulting fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame scFv linker sequence.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

As used herein, the phrase “subject that would benefit from administration of a binding molecule” includes subjects, such as mammalian subjects, that would benefit from administration of a binding molecule used, e.g., for detection of an antigen recognized by a binding molecule (e.g., for a diagnostic procedure) and/or from treatment with a binding molecule to reduce or eliminate the target recognized by the binding molecule. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As described in more detail herein, the binding molecule can be used in unconjugated form or can be conjugated, e.g., to a detectable moiety, a drug, prodrug, or an isotope.

The term “Tm”, also referred to as the “transition temperature”, is the temperature at which 50% of a macromolecule, e.g., binding molecule, becomes denatured, and is considered to be the standard parameter for describing the thermal stability of a protein.

The term “T50” or “T50 value” is the temperature at which 50% of a sample containing a macromolecule, e.g., binding molecule, retain its antigen binding activity following a thermal challenge event as described in US Patent Application No 20080050370.

As used herein the term “scaffolding residue” refers to amino acid residues or residue positions that are not in an interface (e.g., the VH/VL interface) but that are important in maintaining the interface. These amino acid residues do not physically interact with the interface residues on the opposing domain or contribute surface area to the interface, but are nonetheless important for providing proper structural context for interface residues. Such amino acid residues scaffold the interaction between VH and VL.

Two or more amino acid residue positions within a candidate polypeptide sequence that normally occur together are said to “covary” (“covarying residue positions” or “covariant residue positions”). Covariance between two or more amino acid positions is observed when the type of amino acid found at a first amino acid position is dependent on the type of amino acid found at another amino acid position. That is, when one particular amino acid is found at a first position within a sequence, a second particular amino acid is usually found at a second position within the sequence.

As used herein the term “Ig fold” includes a protein domain found in proteins belonging to the immunoglobulin superfamily of proteins. As is well known in the art, the Ig fold is a distinguishing feature of the immunoglobulin superfamily (see, e.g. Bork, P., Holm, L. & Sander, C. 1994. The Immunoglobulin Fold. J. Mol. Biol. 242, 309-320).

As used herein, the term “produced at commercial scale” refers to the production of a binding molecule in a host cell in at least 10 liters of culture media (e.g., at least 20 liters, at least 50 liters, at least 75 liters, at least 100 liters, at least 200 liters, at least 500 liters, at least 1000 liters, at least 2000 liters, at least 5,000 liters, or at least 10,000 liters of culture media).

### II. Multivalent CD23 Binding Molecules

In certain aspects the invention provides multivalent CD23 binding molecules which are capable of cross-linking two or more CD23 molecules (e.g., two, three, four, five, six, or more CD23 target molecules) on the surface of a mammalian (e.g., human) immune cell (e.g., a B cell lymphomas such as a CLL cell). The multivalent CD23 binding molecules of the invention are thus capable inter alia of forming cross-linked CD23 receptor complexes on the surface on an immune cell (e.g., CLL tumor cells). Because experimental studies indicate that cross-linking CD23 molecules on the surface of CLL cells is a necessary step for inducing an apoptotic/cellular death signal, the multivalent CD23 binding molecules of the invention provide a means for enhancing apoptotic signaling. The biological consequence of contacting a multivalent CD23 binding molecule to a CD23 target expressed on an immune cell (e.g., a CLL cell) is an increased level of receptor cross-linking and more efficacious induction of apoptosis/cellular death. This improvement in apoptotic signaling is expected to translate into clinical benefit by providing a more potent/efficacious agent.

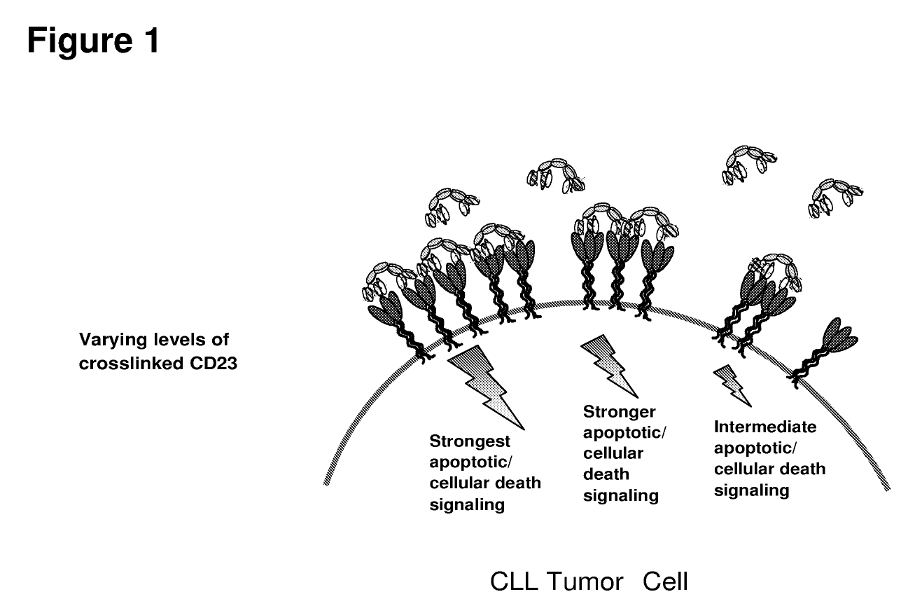

FIG. 1 exemplifies how a multivalent CD23 binding molecule would enhance induction of apoptosis/cellular death in a target immune cell (e.g. a CLL tumor cell). As depicted, the binding molecule is capable of hyper-crosslinking CD23 receptors located on the cell surface forming CD23 receptor complexes. For example, overall, a greater level of CD23 receptor crosslinking occurs upon contact with a multivalent CD23 binding molecule (e.g., a tetravalent CD23 antibody (four binding sites)) than that with a bivalent antibody (two binding sites). Consequently the multivalent antibody would trigger a stronger apoptotic/cellular death signal. Crosslinking such multivalent binding molecules can further enhance signalling and apoptosis.

In certain embodiments, the multivalent CD23 binding molecules of the invention have more than two binding sites. For example, the binding molecules of the invention may be trivalent (three binding sites), tetravalent (four binding sites), pentavalent (five binding sites) hexavalent (six binding sites), heptavalent (seven binding sites), or of higher order valency (e.g., octavalent (eight binding sites) or decavalent (ten binding sites). In certain embodiments, all of the binding sites are capable of specifically binding CD23 molecules. In other embodiment, at least one of the binding sites has a different binding specificity. For example, in certain embodiments, a multivalent CD23 binding molecule is capable of inducing formation of a heteromeric receptor complex comprising at least two CD23 molecules together with an additional distinct immune cell surface molecule (e.g., a CD20 molecule). Use of a multivalent CD23 binding molecule of the invention to induce formation of a heteromeric receptor complex may lead to more complex cell signaling and may more effectively limit immune cell growth.

The improved efficacy of the multivalent CD23 binding molecules of the invention can be determined by comparing the efficacy of the multivalent CD23 binding molecule with that of a suitable control. Suitable controls include monovalent or bivalent CD23 monoclonal antibodies. In certain embodiments, the control is a dimer formed by the crosslinking of two bivalent CD23 monoclonal antibodies with a crosslinker. In certain embodiments, efficacy of the binding molecules of the invention is determined by measuring the degree of cell apoptosis that is induced by the binding molecule. The binding molecules of the invention typically induce apoptosis of immune cells to a greater extent than the control. Several art-recognized methods for measuring cell apoptosis are known in the art. For example, apoptosis can be measured by TUNEL (Terminal deoxynucleotidyl Transferase Biotin-dUTP Nick End Labeling) and other, commercially-available, DNA fragmentation assays (e.g., Apoptag™, Oncor, Gaithersburg, Md.). Alternatively, apoptosis can be assessed using flow cytometric procedures (see, e.g., Nicoletti et al., J. Immunol. Methods, (1991), 139: 271; Illera et al., J. Immunol., (1993), 151: 2965); Dive et al., Biochim. Biophys. Acta., (1992), 1133:275). In other embodiments, apoptosis can be evaluated by assessing modulation of one or more apoptotic factors. For example, cleavage of PARP or a caspase (e.g., caspase 3) can be assessed. In yet other embodiments, efficacy of the binding molecules of the invention can be assessed by measuring immune cell growth. Numerous art-recognized methods for measuring immune cell growth are known in the art (e.g., pulse-chase DNA synthesis assays which measure the rate of [³H]-thymidine incorporation by immune cells). In exemplary embodiments, an improvement in efficacy of at least 10% (e.g., 20%, 30%, 40%, 50%, 60%, 70%, 80%, 90% or more) is observed. In more preferred embodiments a 2-fold, 3-fold, 4-fold, 5-fold, 6-fold, 7-fold, 8-fold, 9-fold, 10-fold, or 15-fold or more increase in efficacy is observed.

In one exemplary embodiment, the binding molecules of the invention induce at least 10% DNA fragmentation (e.g., 20%, 30%, 40%, 50%, 60%, 70%, 80%, 90% or more) of an immune cell as determined by a DNA fragmentation assay, wherein the immune cell (e.g., a CLL cell) is contacted with a suitable amount of the binding molecule (e.g., 1 ug/ml or more, 2 ug/ml or more, 5 ug/ml or more, 10 ug/ml or more, 15 ug/ml or more, or 20 ug/ml or more) under suitable in vitro conditions. In another exemplary embodiment, the binding molecules of the invention induce at least 10% PARP cleavage (e.g., 20%, 30%, 40%, 50%, 60%, 70%, 80%, 90% or more) in an immune cell (e.g., a CLL cell) as determined by a PARP cleavage assay, wherein the immune cell is contacted with a suitable amount of the binding molecule (e.g., 1 ug/ml or more, 2 ug/ml or more, 5 ug/ml or more, 10 ug/ml or more, 15 ug/ml or more, or 20 ug/ml or more) under suitable in vitro conditions. In yet another exemplary embodiment, the binding molecules of the invention induce at least 10% caspase cleavage (e.g., 20%, 30%, 40%, 50%, 60%, 70%, 80%, 90% or more) in an immune cell (e.g., a CLL cell) as determined by a caspase cleavage assay, wherein the immune cell is contacted with a suitable amount of the binding molecule (e.g., 1 ug/ml or more, 2 ug/ml or more, 5 ug/ml or more, 10 ug/ml or more, 15 ug/ml or more, or 20 ug/ml or more) under suitable in vitro conditions.

The binding molecules of the invention comprise at least two binding sites (and preferably at least three or four binding sites) which bind to CD23. Exemplary binding sites include antigen binding sites of a CD23 antibody or the receptor binding sites of a CD23 ligand (e.g., IgE). Other exemplary binding sites include antibody fragments or scFv molecules (e.g., stabilized CD23 scFv molecules described infra). In one embodiment, a CD23 binding molecule of the invention comprises at least four binding sites specific for a CD23 molecule, wherein at least two of the binding sites are antigen binding sites derived from a CD23 antibody, and at least two of the binding sites are scFv molecules (e.g., stabilized CD23 scFv molecules described infra).

In certain embodiments, the CD23 binding molecules of the invention comprise binding sites having the same binding specificity. In other embodiments, at least two of the binding sites have different binding specificities. In one embodiment, said binding sites bind different epitopes on the same CD23 molecule. In other embodiments, said binding sites bind different CD23 molecules. In other embodiments, the binding sites bind different epitopes on different CD23 molecules. In other embodiments, at least one binding site binds a CD23 molecule and at least one binding site binds another molecule on the surface of the immune cell (e.g., CD20).

In certain embodiments, the CD23 binding molecules of the invention have apparent CD23 binding affinities ranging from 0.1 nM to 1000 nM, more preferably at least 50 nM, still more preferably at least 5 nM, and most preferably at least 0.5 nM.

In certain embodiments, the CD23 binding molecules of the invention are multimeric molecules. In one embodiment, the CD23 binding molecules are dimers. In one embodiment, the dimers of the invention are homodimers, comprising two identical monomeric subunits. In another embodiment, the dimers of the invention are heterodimers, comprising two non-identical monomeric subunits. The dimers comprise at least two polypeptide chains. In one embodiment, the CD23 binding molecules comprise two polypeptide chains. In another embodiment, the CD23 binding molecules comprise three polypeptide chains. In another embodiment, the CD23 binding molecules comprise four polypeptide chains.

In one embodiment, a binding molecule of the invention comprises at least one CDR of a CD23 antibody, e.g., an antibody known in the art to bind to CD23. Said CD23 antibody may be a chimeric antibody, a humanized antibody, a fully human antibody, or a primatized antibody. In another embodiment, a CD23 binding molecule of the invention comprises at least two CDRs of a CD23 antibody. In another embodiment, a binding molecule of the invention comprises at least three CDRs of a CD23 antibody. In another embodiment, a CD23 binding molecule of the invention comprises at least four CDRs of a CD23 antibody. In another embodiment, a CD23 binding molecule of the invention comprises at least five CDRs of a CD23 antibody. In another embodiment, a CD23 binding molecule of the invention comprises at least six CDRs of a CD23 antibody. In a preferred embodiment, a CD23 binding molecule of the invention comprises at least one VH domain of a CD23 antibody, e.g., an antibody known in the art to bind to CD23. In a preferred embodiment, a binding molecule of the invention comprises at least one VL domain of a CD23 antibody. In another preferred embodiment, a CD23 binding molecule of the invention comprises at least one VH domain and one VL domain of a CD23 antibody.

In certain embodiments, a CD23 binding molecule of the invention binds to the same epitope as a 5E8 antibody. In another embodiment, a CD23 binding molecule of the invention comprises a binding site derived from a 5E8 antibody. For example, the binding molecule may comprise a binding site having at least one CDR (e.g., at least 1, 2, 3, 4, 5, or 6 CDRs) derived from a 5E8 antibody. In exemplary embodiments, the 5E8 antibody is PRIMATIZED® p5E8G1 antibody. PRIMATIZED® p5E8G1 antibody is a chimeric macaque/human (PRIMATIZED®) monoclonal antibody containing macaque heavy and light variable regions fused to human gamma 1 and kappa constant regions, respectively (see, e.g., U.S. Pat. No. 6,011,138, which is incorporated by reference herein). In other embodiments, a CD23 binding molecule of the invention comprises a binding site derived from an art-recognized CD23 antibody selected from the group consisting of a 6G5 antibody (e.g., a PRIMATIZED® p6G6G1 antibody), a 2C8 antibody, a B3B11 antibody, and a 3G12 antibody (see e.g., U.S. Pat. No. 6,011,138). Reference heavy and light chain variable sequences for certain of these antibodies are provided in Table 12 and Table 13 below. Additional exemplary binding molecules of the invention may comprise CD23 binding sites derived from an antibody expressed by one or more of the following cell lines or hybridomas deposited with the ATCC: (a) CRL-1596, (b) CRL-1923, (c) CRL-2407, (d) CRL-2408, (e) CRL-2625, (f) CRL-2630, (g) CRL-2631, (h) CRL-2632, (i) CRL-3000, (j) CRL-3002, (k) CRL-3003, (l) CRL-3004, and (m) CRL-3005.

In another embodiment, the CD23 binding molecule binds the same epitope as the art-recognized antibody. In yet other embodiments, the CD23 binding molecule cross-blocks the art-recognized antibody. Such cross-blocking can be determined by art-recognized competition assays including, for example, surface plasmon resonance (SPR)-based competition assays. Other art-recognized CD23 antibodies from which the CD23 binding molecules of the invention may be derived are described U.S. Pat. No. 7,223,392; U.S. Pat. No. 7,033,589; U.S. Pat. No. 7,008,623; U.S. Pat. No. 6,893,636; U.S. Pat. No. 6,627,195; U.S. Pat. No. 6,011,138; US Patent Publication No. 20070065435; US Patent Publication No. 20070065434; US Patent Publication No. 20060073147; or US Patent Publication No. 20050118175; each of which is incorporated by reference herein in its entirety.

TABLE 12

Reference VH-CDR1, VH-CDR2, and VH-CDR3 amino acid
sequences*

| | VH SEQUENCE (VH-CDR1, | | | |
|---|---|---|---|---|
| | VH-CDR2, and VH-CDR3 | | | |
| Antibody | underlined) | VH CDR1 | VH CDR2 | VH CDR3 |
| Primate 6G6 | QLQLQESGPGVVKPSETLSLTCAV | SSNWWT | RISGSGG | DWAQIA |
| | SGGSVSSSNWWTWIRQPPGKGLE | (SEQ ID | ATNYNP | GTTLGF |
| | WIGRISGSGGATNYNPSLKSRVIIS | NO:86) | SLKS | (SEQ ID |
| | QDTSKNQFSLNLNSVTAADTAVY | | (SEQ ID | NO:88) |
| | YCARDWAQIAGTTLGFWGQGVLV | | NO:87) | |
| | TVSS | | | |
| | (SEQ ID NO:85) | | | |
| Primate 5E8 | EVQLVESGGGLAKPGGSLRLSCAA | FNNYYM | RISSSGD | LTTGSDS |
| | SGFRFTFNNYYMDWVRQAPGQGL | D | PTWYAD | (SEQ ID |
| | EWVSRISSSGDPTWYADSVKGRFT | (SEQ ID | SVKG | NO:92) |
| | ISRENAKNTLFLQMNSLRAEDTAV | NO:90) | (SEQ ID | |
| | YYCASLTTGSDSWGQGVLVTVSS | | NO:91) | |
| | (SEQ ID NO:89) | | | |

*Determined by the Kabat system (see supra).
N = nucleotide sequence, P = polypeptide sequence.

TABLE 13

Reference VL-CDR1, VL-CDR2, and VL-CDR3 amino acid
sequences*

| | VL SEQUENCE PN/PP (VL-CDR1, | | | |
|---|---|---|---|---|
| | VL-CDR2, and VL-CDR3 | | | |
| Antibody | sequences underlined) | VL CDR1 | VL CDR2 | VL CDR3 |
| Primate 6G6 | QSAPTQPPSVSGSPGQSVTISCTGT | TGTSDDV | DVAKRAS | CSYTTSS |
| | SDDVGGYNYVSWYQHHPGKAPK | GGYNYVS | (SEQ ID | TLL |
| | LMIYDVAKRASGVSDRFSGSKSG | (SEQ ID | NO:95) | (SEQ ID |
| | NTAYCCSYTTSSTLLFGRGTRLTVL | NO:94) | | NO:96) |
| | (SEQ ID NO:93) | | | |
| Primate 5E8 | DIQMTQSPSSLSASVGDRVTITCR | RASQDIR | VASSLQS | LQVYST |
| | ASQDIRYYLNWYQQKPGKAPKLL | YYLN | PRT | PRT |
| | IYVASSLQSGVPSRFSGSGSGTEFT | (SEQ ID | (SEQ ID | (SEQ ID |
| | LTVSSLQPEDFATYYCLQVYSTPR | NO:98) | NO:99) | NO:100) |
| | TFGQGTKVEIK | | | |
| | (SEQ ID NO:97) | | | |

*Determined by the Kabat system (see supra).

In preferred embodiments, multivalent binding molecules of the invention are constructed by linking one or more scFv molecules (e.g., stabilized CD23 scFv molecules described infra) to a monovalent or bivalent CD23 binding molecule, e.g. an art-recognized CD23 antibody, e.g., a 5E8 antibody. scFv molecules may be linked in series to the CD23 binding molecule or they may be attached to either the N- or C-terminus of a CD23 binding polypeptide, e.g., either the light or heavy chain of a CD23 antibody.

Exemplary forms of multivalent CD23 binding molecules of the invention are set forth Section VI infra.

### III. Stabilized CD23 Binding Molecules

a. Stabilized CD23 scFv Molecules

In one certain aspect, a binding molecule of the invention is a stabilized CD23 scFv molecule. The stabilized CD23 scFv molecules of the invention may comprise an scFv linker interposed between a V_{H} domain and a V_{L} domain, wherein the V_{H} and V_{L} domains are linked by a disulfide bond between an amino acid in the V_{H} and an amino acid in the V_{L} domain. In other embodiments, the stabilized CD23 scFv molecules of the invention comprise an scFv linker having an optimized length or composition. In yet other embodiments, the stabilized CD23 scFv molecules of the invention comprise a V_{H} or V_{L} domain having at least one stabilizing amino acid substitution(s). In yet other embodiments, a stabilized CD23 scFv molecule of the invention comprises at least two of the above listed stabilizing features (e.g., a stabilizing disulfide bond and a stabilizing amino acid substitution). In yet other embodiments, a stabilized CD23 scFv molecule of the invention comprises all of the above listed stabilizing features. Methods for identifying or making stabilized CD23 scFv molecules are described in detail in U.S. patent application Ser. No. 11/725,950, filed Mar. 19, 2007, which is incorporated by reference herein in its entirety.

The stabilized CD23 scFv molecules of the invention have improved protein stability. In one embodiment, populations of the stabilized CD23 scFv molecules of the invention or polypeptides comprising the same are expressed as a monomeric, soluble protein of which is no more than 25% in dimeric, tetrameric, or otherwise aggregated form (e.g., less than about 25%, about 20%, about 15%, about 10%, or about 5%). In another embodiment, stabilized CD23 scFv molecules of the invention have a T50 of greater than 55° C. (e.g., 55, 56, 57, 58, 59, 60° C., or more). In more preferred embodiments, stabilized CD23 scFv molecules of the invention have a T50 of greater than 60° C. (e.g., 60, 61, 62, 63, 64, 65° C., or more). In yet more preferred embodiments, stabilized CD23 scFv molecules of the invention have a T50 of greater than 65° C. (e.g., 65, 66, 67, 68, 69, 70° C., or more). In still more preferred embodiments, stabilized CD23 scFv molecules of the invention have a T50 of greater than 70° C. (e.g., 70, 71, 73, 74, 75° C., or more). In other embodiments, stabilized CD23 scFv molecules of the invention have Tm-values greater than 55° C. (e.g., 55, 56, 57, 58, 59° C. or higher), greater than 60° C. (e.g., 60, 61, 62, 63, 64° C. or higher), greater than 65° C. (e.g., 65, 66, 67, 68, 69° C. or higher), or greater than 70° C. (e.g., 71, 72, 73, 74, 75° C. or higher).

The stabilized CD23 scFv molecules of the invention have binding specificity for CD23. The VH and VL domains used to make a stabilized scFv may be derived from the same or from different CD23 antibodies. In another embodiment, a VH or VL for use in a stabilized CD23 scFv of the invention may comprise one or more CDRs which bind to a target of interest, while the remainder of the VH or VL domain is derived from a different antibody or is synthetic. In a preferred embodiment, a stabilized CD23 scFv molecule of the invention comprises at least one CDR of a CD23 antibody, e.g., a CD23 antibody known in the art. In another embodiment, a stabilized CD23 scFv molecule of the invention comprises at least two CDRs of a given CD23 antibody. In another embodiment, a stabilized CD23 scFv molecule of the invention comprises at least three CDRs of a given CD23 antibody. In another embodiment, a stabilized CD23 scFv molecule of the invention comprises at least four CDRs of a given CD23 antibody. In another embodiment, a stabilized CD23 scFv molecule of the invention comprises at least five CDRs of a given CD23 antibody. In another embodiment, a stabilized CD23 scFv molecule of the invention comprises at least six CDRs of a given CD23 antibody. In a preferred embodiment, a stabilized CD23 scFv molecule of the invention comprises at least one VH domain of a CD23 antibody, e.g., a CD23 antibody known in the art. In a preferred embodiment, a stabilized CD23 scFv molecule of the invention comprises at least one VL domain of a given CD23 antibody. In another preferred embodiment, a stabilized CD23 scFv molecule of the invention comprises at least one VH domain and one VL domain of a CD23 antibody known in the art. CD23 scFv molecules can be constructed in a VH-linker-VL orientation or VL-linker-VH orientation.

In certain embodiments, a stabilized CD23 scFv molecule of the invention binds to the same epitope as a 5E8 antibody. In another embodiment, a stabilized CD23 scFv molecule of the invention cross-blocks (or competes with) binding of the 5E8 antibody to the epitope. In another embodiment, a stabilized CD23 scFv molecule of the invention comprises at least one CDR (e.g., at least 1, 2, 3, 4, 5, or 6 CDRs) from a 5E8 antibody. In exemplary embodiments, the 5E8 antibody is PRIMATIZED® p5E8G1 antibody. In other embodiments, a stabilized CD23 scFv molecule of the invention comprises a binding site derived from an art-recognized CD23 antibody selected from the group consisting of a 6G5 antibody, a 2C8 antibody, a B3B11 antibody, and a 3G12 antibody. In yet other embodiments, a stabilized CD23 scFv molecule binds the same epitope as the art-recognized antibody. In still other embodiments, a stabilized CD23 scFv molecule cross-blocks (or competes with the binding of) the art-recognized antibody. Other art-recognized CD23 antibodies from which the stabilized CD23 scFv molecules of the invention may be derived are described U.S. Pat. No. 7,223,392; U.S. Pat. No. 7,033,589; U.S. Pat. No. 6,893,636; U.S. Pat. No. 6,011,138; US Patent Publication No. 20070065435; US Patent Publication No. 20070065434; US Patent Publication No. 20060073147; or US Patent Publication No. 20050118175; each of which is incorporated by reference herein in its entirety.

The stability of CD23 scFv molecules of the invention or fusion proteins comprising them can be evaluated in reference to the biophysical properties (e.g., thermal stability) of a conventional (non-stabilized) CD23 scFv molecule or a CD23 binding molecule comprising a conventional CD23 scFv molecule. In one embodiment, the CD23 binding molecules of the invention have a T50 that is greater than about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C. than a control binding molecule (e.g. a conventional scFv molecule).

In exemplary embodiments, the stabilized CD23 scFv molecules of the invention comprise four or more stabilizing mutations (e.g., 4, 5, 6, 7, 8, or more stabilizing mutations) within one or more variable domains (VH and/or VL) of the scFv. In one embodiment, the stabilized CD23 scFv molecules of the invention comprise four or more stabilizing mutations (e.g., 4, 5, 6, 7, 8, or more stabilizing mutations) which are independently selected from the group consisting of:
a) substitution of an amino acid (e.g., glutamic acid) at Kabat position 6 of VH, e.g., with glutamine;
b) substitution of an amino acid (e.g., asparagine) at Kabat position 32 of VH, e.g., with serine;
c) substitution of an amino acid (e.g., serine) at Kabat position 49 of VH, e.g., with glycine or alanine;
d) substitution of an amino acid (e.g., proline) at Kabat position 56 of VH, e.g., with a histidine;
e) substitution of an amino acid (e.g., glutamic acid) at Kabat position 72 of VH, e.g., with aspartate;
f) substitution of an amino acid (e.g., valine) at Kabat position 50 of VL, e.g., with serine, aspartic acid, or glutamic acid;
g) substitution of an amino acid (e.g., valine) at Kabat position 75 of VL, e.g., with isoleucine; and
h) substitution of an amino acid (e.g., phenylalanine) at Kabat position 83 of VL, e.g., with serine, alanine, glycine, or threonine.

In one exemplary embodiment, a stabilized CD23 scFv molecule of the invention comprises at least one (e.g., 1, 2, or 3) of stabilizing mutations (a), (c), or (f) above (e.g., at least (a) and (c), at least (a) and (f), at least (c) and (f); and at least (a), (c), and (f)). In another exemplary embodiment, a stabilized CD23 scFv molecule of the invention comprises at least one (e.g., 1, 2, 3, 4, or 5) of stabilizing mutations (a), (b), (c), (d), or (h) above.

In an exemplary embodiment, a stabilized CD23 scFv molecule of the invention comprises substitutions (a), (b), (c), (d), (f), and (h). In another exemplary embodiment, a stabilized CD23 scFv molecule of the invention comprises substitutions (a), (b), (c), (d), (f), and (g). In another exemplary embodiment, a stabilized CD23 scFv molecule of the invention comprises substitutions (a), (c), (d), (f), and (h). In another exemplary embodiment, a stabilized CD23 scFv molecule of the invention comprises substitutions (a), (b), (c), (d), (f), and (g). In another exemplary embodiment, a stabilized CD23 scFv molecule of the invention comprises substitutions (a), (b), (c), (d), (f), and (h). In another exemplary embodiment, a stabilized CD23 scFv molecule of the invention comprises substitutions (a), (b), (c), (e), (f), and (h). In another exemplary embodiment, a stabilized CD23 scFv molecule of the invention comprises substitutions (a), (c), (e), (f), and (g).

In yet another exemplary embodiment, a stabilized CD23 scFv molecule of the invention comprises a sequence set forth as SEQ ID NO:10. In yet another exemplary embodiment, a stabilized CD23 scFv molecule of the invention comprises a sequence set forth as SEQ ID NO:12.

Thermal stability of the stabilized CD23 scFv molecules of the invention can be measured using methods known in the art. For example, in one embodiment, Tm can be measured. Methods for measuring Tm and other methods of determining protein stability are described in detail in U.S. patent application Ser. No. 11/725,950, filed Mar. 19, 2007, which is incorporated by reference herein in its entirety. In certain embodiments, the expression levels (e.g., as measured by % yield) of the compositions of the invention are evaluated. In other preferred embodiments, the aggregation levels of the compositions of the invention are evaluated. In certain embodiments, the stability properties of a composition of an invention are compared with that of a suitable control. Exemplary controls include conventional scFv molecules. A particularly preferred control is a (Gly₄Ser)₃ scFv molecule.

In one embodiment, one or more art-recognized parameters (e.g., thermal stability, % aggregation, % yield, % loss, % proteolysis, or binding affinity) are measured. In one embodiment, one or more of these parameters is measured following expression in a mammalian cell. In one embodiment, one or more of these parameters are measured under large scale manufacturing conditions (e.g., expression of scFvs or molecules comprising scFvs in a bioreactor).

In one embodiment, thermal stability of the compositions of the invention may be analyzed using a number of non-limiting biophysical or biochemical techniques known in the art. In certain embodiments, thermal stability is evaluated by analytical spectroscopy (e.g., Differential Scanning Calorimetry (DSC), Circular Dichroism (CD) spectroscopy; Fluorescence Emission; Nuclear Magnetic Resonance (NMR) spectroscopy). In other embodiments, the thermal stability of a composition of the invention is measured biochemically. An exemplary biochemical method for assessing thermal stability is a thermal challenge assay (see U.S. patent application Ser. No. 11/725,950, filed Mar. 19, 2007, which is incorporated by reference herein in its entirety).

In one embodiment, a stabilized CD23 scFv molecule of the invention comprises a scFv linker consisting of or comprising the amino acid sequence (Gly₄Ser)₄ sequence. Other exemplary linkers comprise or consist of (Gly₄Ser)₃ and (Gly₄Ser)₅ sequences. scFv linkers can be of varying lengths. In one embodiment, the scFv linker is from about 5 to about 50 amino acids in length. In another embodiment, the scFv linker is from about 10 to about 40 amino acids in length. In another embodiment, the scFv linker is from about 15 to about 30 amino acids in length. In another embodiment, an scFv linker of the invention is from about 17 to about 28 amino acids in length. In another embodiment, the scFv linker is from about 19 to about 26 amino acids in length. In another embodiment, the scFv linker is from about 21 to about 24 amino acids in length.

In certain embodiments, the stabilized CD23 scFv molecules of the invention comprise at least one disulfide bond which links an amino acid in the VL domain with an amino acid in the VH domain. Cysteine residues are necessary to provide disulfide bonds. Disulfide bonds can be included in a CD23 scFv molecule of the invention, e.g., to connect FR4 of VL and FR2 of VH or to connect FR2 of VL and FR4 of VH. Exemplary positions for disulfide bonding include: 43, 44, 45, 46, 47, 103, 104, 105, and 106 of VH and 42, 43, 44, 45, 46, 98, 99, 100, and 101 of VL, Kabat numbering. Exemplary combinations of amino acid positions which are mutated to cysteine residues include: VH44-VL100, VH105-VL43, VH105-VL42, VH44-VL101, VH106-VL43, VH104-VL43, VH44-VL99, VH45-VL98, VH46-VL98, VH103-VL43, VH103-VL44, and VH103-VL45. In one embodiment, a disulfide bond links V_{H} amino acid 44 and V_{L} amino acid 100.

Modifications of the genes which encode the VH and VL domains may be accomplished using techniques known in the art, for example, site-directed mutagenesis. In one embodiment, a stabilized CD23 scFv molecule of the invention comprises an scFv linker having the amino acid sequence (Gly₄ Ser)₄ interposed between a V_{H} domain and a V_{L} domain, wherein the V_{H} and V_{L} domains are linked by a disulfide bond between an amino acid in the V_{H} at amino acid position 44 and an amino acid in the V_{L} at amino acid position 100.

In another exemplary embodiment, a stabilized CD23 scFv molecule of the invention comprises one or more of the stabilizing amino acid substitutions described herein and an scFv linker with an optimized length or composition (e.g. (Gly₄Ser)₄). In another exemplary embodiment, a stabilized CD23 scFv molecule of the invention comprises one or more of the amino acid substitutions described herein and a disulfide bond which links an amino acid in the VL domain with an amino acid in the VH domain (e.g. VH44-VL100). In yet another exemplary embodiment, a stabilized CD23 scFv molecule of the invention comprises one or more of the amino acid substitutions described herein, an scFv linker with an optimized length or composition (e.g. (Gly₄Ser)₄), and a disulfide bond which links an amino acid in the VL domain with an amino acid in the VH domain (e.g. VH44-VL100).

Stabilized CD23 scFv molecules may be expressed using art recognized techniques. For example, in one embodiment, such molecules may be expressed using an expression vector appropriate for expression in a cellular expression system, e.g., a bacterial or mammalian expression system. In one embodiment, CD23 scFv molecules may be expressed in E. coli, e.g., using a vector appropriate for periplasmic expression. Additional sequences may be included to optimize expression, e.g., a signal sequence and/or a tag to facilitate purification and/or detection of the scFv.

b) Stabilized Binding Molecules Comprising Stabilized CD23 scFv Molecules

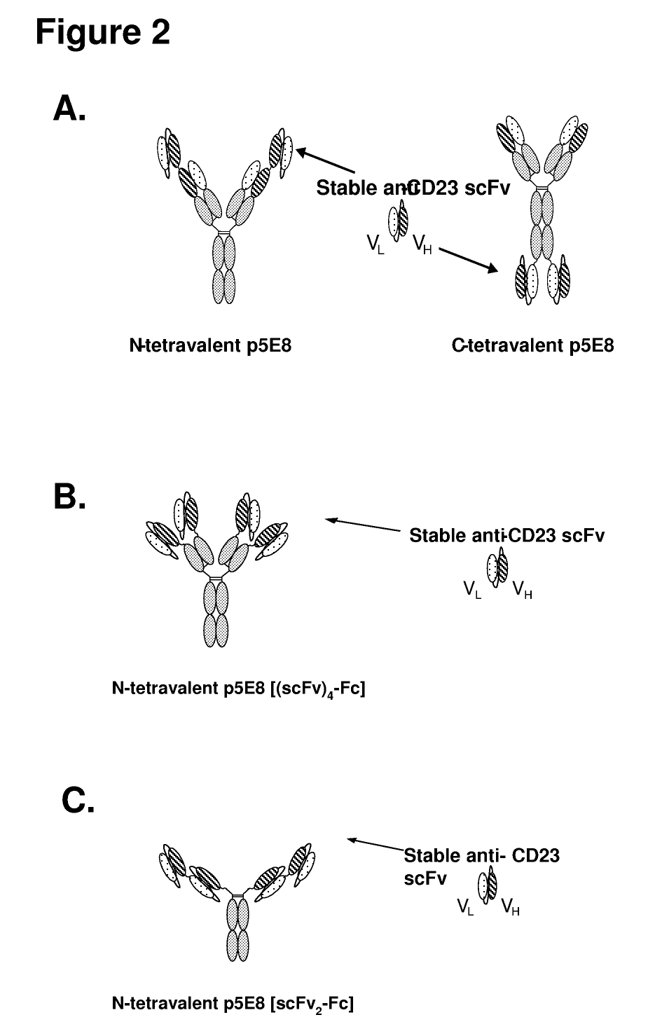

In certain aspects, the invention provides stabilized binding molecules comprising the stabilized CD23 scFv molecules of the invention. For example, the stability of binding molecules (e.g., CD23 binding molecules) can be enhanced by incorporating or appending a stabilized scFv molecule of the invention to the binding molecule. For example, stability engineered scFv molecules of the invention can be used as key building blocks for constructing multivalent CD23 binding molecules (e.g., multivalent CD23 binding molecules of the invention supra). FIG. 2 shows a schematic diagram of different forms of a stabilized tetravalent CD23 antibody of the invention. The stability-engineered scFv can be appended as a gene fusion to either the carboxy or amino terminus of light or heavy chain of a CD23 antibody (e.g., a 5E8 antibody) (see FIG. 2A). In another embodiment, stabilized scFv molecules can be directly fused to the N-terminus of one or both CH1 domains and/or the N-terminus of one or both CL domains of an antibody. Said scFv molecules can replace one or all of the VL and VH domains of an antibody (see FIG. 2B). In yet other embodiments, stabilized scFv molecules can be directly fused in series to the N-terminus of an Fc region or portion thereof (see FIG. 2C).

It is conceivable that antibodies consisting of greater than four binding sites can be engineered by adding one or more stability-engineered CD23 scFvs to both the carboxy and amino termini to form a hexavalent antibody or by adding the stabilized scFv with one or more stabilized or conventional scFv molecules in series to form higher order valencies (e.g. 6, 8, 10, etc. binding sites).

Stabilized CD23 scFv molecules may be incorporated into binding molecules using protein conjugation methodology that is known in the art. In one embodiment, the stabilized CD23 scFv is fused directly to an N- or C-terminus of a binding polypeptide, e.g., a CD23 antibody molecule. In another embodiment, a non-peptide linker is employed to link the stabilized CD23 scFv to an N- or C-terminus of a binding polypeptide, e.g., a CD23 antibody molecule. In yet other embodiments, a connecting peptide is used to link the stabilized CD23 scFv to the binding polypeptide. In an exemplary embodiment, the connecting peptide is a short gly/ser rich peptide. Exemplary Gly/Ser rich peptides comprise or consist of the (Gly₄Ser)₅ or Ser(Gly₄Ser)₃ sequence. Other exemplary connecting peptides are known in the art (see, e.g., International PCT Application Nos. WO 2005/000898 and WO 2005/000899). In one embodiment, a stabilized CD23 scFv is linked to the C-terminal end of a binding molecule, e.g., a CD23 antibody molecule, using a S(G₄S)₃ linker. In another embodiment, a stabilized scFv of the invention is linked to the N-terminal end of a binding molecule, e.g., a CD23 antibody molecule, using a (G₄S)₅ linker.

In one embodiment, at least one stabilized CD23 scFv molecule is appended to an antibody molecule having a different binding specificity (e.g., a CD23 antibody which binds a different epitope of CD23) to make a bispecific CD23 molecule. In another embodiment, two stabilized CD23 scFv molecules are appended to an antibody molecule to make a bispecific CD23 molecule.

In certain embodiments, CD23 binding molecules of the invention result in reduced aggregation as compared to conventional CD23 scFv molecules or CD23 binding molecules comprising conventional scFv molecules. In one embodiment, a stabilized CD23 binding molecule produced by the methods of the invention has a decrease in aggregation of at least 1% relative to the unstabilized CD23 binding molecule. In other embodiments, the stabilized CD23 binding molecule has a decrease in aggregation of at least 2%, at least 5%, at least 10%, at least 20%, at least 30%, at least 50%, at least 75%, or at least 100%, relative to the unstabilized binding molecule.

In other embodiments, CD23 binding molecules of the invention result in increased long-term stability or shelf-life as compared to conventional CD23 scFv molecules or CD23 binding molecules comprising conventional CD23 scFv molecules.

In one embodiment, a stabilized CD23 binding molecule produced by the methods of the invention has an increase in shelf life of at least 1 day relative to the unstabilized binding molecule. This means that a preparation of CD23 binding molecules has substantially the same amount of stable CD23 binding molecules as present on the previous day. In other embodiments, the stabilized CD23 binding molecule has an increase in shelf life of at least 2 days, at least 5 days, at least 1 week, at least 2 weeks, at least 1 month, at least 2 months, at least 6 months, or at least 1 year, relative to the unstabilized binding molecule.

In certain embodiments, stabilized CD23 binding molecules of the invention are expressed at increased yield as compared to unstabilized CD23 binding molecules (e.g., conventional CD23 scFv molecules or CD23 binding molecules comprising conventional CD23 scFv molecules). In one embodiment, a stabilized CD23 binding molecule of the invention has an increase in yield of at least 1% relative to the unstabilized CD23 binding molecule. In other embodiments, the stabilized CD23 binding molecule has an increase in yield of at least 2%, at least 5%, at least 10%, at least 20%, at least 30%, at least 50%, at least 75%, at least 80%, at least 90%, at least 95%, at least 98% or at least 100%, relative to the unstabilized binding molecule.

In exemplary embodiments, CD23 binding molecules of the invention are expressed at increased yields (as compared to conventional CD23 scFv molecules or binding molecules comprising conventional scFv molecules) in a host cell, e.g., a bacterial or eukaryotic (e.g., yeast or mammalian) host cell. Exemplary mammalian host cells include Chinese Hamster Ovary (CHO) cells, HELA (human cervical carcinoma) cells, CVI (monkey kidney line) cells, COS (a derivative of CVI with SV40 T antigen) cells, R1610 (Chinese hamster fibroblast) cells, BALBC/3T3 (mouse fibroblast) cells, HAK (hamster kidney line) cells, SP2/O (mouse myeloma) cells, BFA-1c1BPT cells (bovine endothelial cells), RAJI (human lymphocyte) cells, PER.C6® (human retina-derived cell line, Crucell, The Netherlands) and 293 cells (human kidney). In a preferred embodiment, two stabilized CD23 scFv molecules are appended to an antibody molecule to create a stabilized CD23 binding molecule for secretion in CHO cells.

In other embodiments, host cells capable of expressing stabilized CD23 binding molecules can be screened to select for single cell isolates that are capable of expressing high levels of solubilized and properly-folded stabilized CD23 binding molecules (e.g. binding molecules exhibiting less than 10% aggregation). Such methods may employ fluorescence-activated cell sorting (FACS) techniques (see, for example, Brezinky et al., J Immunol Meth (2003). 277:141-155). In one embodiment, the single cell isolate is adapted to serum-free conditions to establish a stable producer cell line. The stable producer cell line may then be cultured to facilitate large-scale manufacture of a stabilized binding molecule of the invention.

In other embodiments, the CD23 binding molecules of the invention are expressed at increased yields (relative to an unstabilized CD23 binding molecule) in a host cell under large-scale (e.g., commercial scale) conditions. In exemplary embodiments, the CD23 binding molecule have increased yield when expressed in at least 10 liters of culture media. In other embodiments, a stabilized CD23 binding molecule has an increase in yield when expressed from a host cell in at least 20 liters, at least 50 liters, at least 75 liters, at least 100 liters, at least 200 liters, at least 500 liters, at least 1000 liters, at least 2000 liters, at least 5,000 liters, or at least 10,000 liters of culture media. In an exemplary embodiment, at least 10 mg (e.g., 10 mg, 20 mg, 50 mg, or 100 mg) of a stabilized CD23 binding molecule are produced for every liter of culture media.

In one embodiment, stabilized CD23 binding molecules of the invention comprise at least one stabilized CD23 scFv (e.g. 2, 3, or 4 scFvs) linked to the C-terminus of an antibody heavy. In another embodiment, the stabilized CD23 binding molecules of the invention comprise at least one stabilized CD23 scFv (e.g. 2, 3, or 4 scFvs) linked to the N-terminus of an antibody heavy chain. In another embodiment, the CD23 binding molecules of the invention comprise at least one stabilized CD23 scFv (e.g. 2, 3, or 4 scFvs) linked to the N-terminus of an antibody light chain. In another embodiment, the stabilized CD23 binding molecules of the invention comprise at least one stabilized CD23 scFv (e.g., 2, 3, or 4 scFvs linked to the N-terminus of the heavy chain or light chain and at least one stabilized CD23 scFv (e.g., 2, 3, or 4 scFvs) linked to the C-terminus of the heavy chain.

### VI. Exemplary Forms OF CD23 Binding Molecules

i. CD23 Antibodies

In certain embodiments, CD23 binding molecules of the invention comprise or consist of CD23 antibodies. CD23 antibodies of the present invention can be produced by any method known in the art for the synthesis of antibodies, in particular, by chemical synthesis or preferably, by recombinant expression techniques as described herein. For example, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Yet other embodiments of the present invention comprise the generation of human or substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

In certain embodiments both the variable and constant regions of CD23 antibodies, or antigen-binding fragments, variants, or derivatives thereof are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Polyclonal antibodies to an epitope of interest can be produced by various procedures well known in the art. For example, an antigen comprising the epitope of interest can be administered to various host animals including, but not limited to, rabbits, mice, rats, chickens, hamsters, goats, donkeys, etc., to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal CD23 antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using CD23 knockout mice to increase the regions of epitope recognition. Monoclonal antibodies can be prepared using a wide variety of techniques known in the art including the use of hybridoma and recombinant and phage display technology as described elsewhere herein.

Using art recognized protocols, in one example, antibodies are raised in mammals by multiple subcutaneous or intraperitoneal injections of the relevant antigen (e.g., purified CD23 or cells or cellular extracts comprising CD23) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen. In this well known process (Kohler et al., Nature 256:495 (1975)) the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Those skilled in the art will also appreciate that DNA encoding antibodies or antibody fragments (e.g., antigen binding sites) may also be derived from antibody libraries, such as phage display libraries. In a particular, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, individual Fv regions from light or heavy chains, or disulfide stabilized Fv antibody domains recombinantly fused to either the phage gene III or gene VIII protein. Exemplary methods are set forth, for example, in EP 368 684 B1; U.S. Pat. No. 5,969,108, Hoogenboom, H. R. and Chames, Immunol. Today 21:371 (2000); Nagy et al. Nat. Med. 8:801 (2002); Huie et al., Proc. Natl. Acad. Sci. USA 98:2682 (2001); Lui et al., J. Mol. Biol. 315:1063 (2002) each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nat. Biotechnol. 18:1287 (2000); Wilson et al., Proc. Natl. Acad. Sci. USA 98:3750 (2001); or Irving et al., J. Immunol. Methods 248:31 (2001)). In yet another embodiment, cell surface libraries can be screened for antibodies (Boder et al., Proc. Natl. Acad. Sci. USA 97:10701 (2000); Daugherty et al., J. Immunol. Methods 243:211 (2000)). Yet another exemplary embodiment, high affinity human Fab libraries are designed by combining immunoglobulin sequences derived from human donors with synthetic diversity in selected complementarity determining regions such as CDR H1 and CDR H2 (see, e.g., Hoet et al., Nature Biotechnol., 23:344-348 (2005), which is incorporated herein by reference). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. For example, DNA sequences encoding VH and VL regions are amplified or otherwise isolated from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., an CD23 polypeptide or a fragment thereof) can be selected or identified with antigen, e.g., using labeled antigen or antigen bound or captured to a solid surface or bead.

Additional examples of phage display methods that can be used to make antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988). See also, U.S. Pat. No. 5,565,332.)

Further, antibodies to target polypeptides of the invention can, in turn, be utilized to generate anti-idiotype antibodies that “mimic” target polypeptides using techniques well known to those skilled in the art. (See, e.g., Greenspan & Bona, FASEB J. 7(5):437-444 (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a desired target polypeptide and/or to bind its ligands/receptors, and thereby block its biological activity.

ii. scFv-Containing CD23 Binding Molecules

In one embodiment, the CD23 binding molecules of the invention are binding molecules comprising at least one scFv molecule, e.g. an scFv molecule described supra. In other embodiments, the binding molecules of the invention comprise two scFv molecules. In certain embodiments, the scFv molecule is a conventional scFv molecule. In other embodiments, the scFv molecule is a stabilized scFv molecule described supra. In certain embodiments, a multivalent binding molecule may be created by linking a scFv molecule (e.g., a stabilized scFv molecule described supra) with a parent binding molecule selected from any of the binding molecules described supra. In certain embodiment, a binding molecule of the invention may comprise a scFv molecule linked to a second scFv molecule or a non-scFv binding molecule (e.g., a CD23 antibody). In one embodiment, a binding molecule of the invention is a CD23 antibody (e.g., a 5E8 antibody) to which a stabilized CD23 scFv molecule has been fused. It shall be recognized that scFv-containing CD23 binding molecules of the invention include any of the stabilized scFv-containing CD23 binding molecules disclosed in section III (b) supra, as well corresponding binding molecules in which one or more of the scFvs are conventional scFvs instead of stabilized scFvs.

When a stabilized scFv is linked to a parent binding molecule, linkage of the stabilized scFv molecule preferably improves the thermal stability of the binding molecule by at least about 2° C. or 3° C. In one embodiment, the scFv-containing binding molecule of the invention has a 1° C. improved thermal stability as compared to a conventional binding molecule. In another embodiment, a binding molecule of the invention has a 2° C. improved thermal stability as compared to a conventional binding molecule. In another embodiment, a binding molecule of the invention has a 4, 5, 6° C. improved thermal stability as compared to a conventional binding molecule. In yet other embodiments, a binding molecule of the invention has a 7, 8, 9, 10, 11, 12, 13, 14, 15, 16, 17, 18, 19, 20, 21, 22, 23, 24, 25, 26, 27, 28, 29, 30° C. increase in thermal stability as compared to a conventional binding molecule.

In one embodiment, the binding molecules of the invention are stabilized “antibody” or “immunoglobulin” molecules, e.g., naturally occurring antibody or immunoglobulin molecules (or an antigen binding fragment thereof) or genetically engineered antibody molecules that bind antigen in a manner similar to antibody molecules and that comprise an scFv molecule of the invention. As used herein, the term “immunoglobulin” includes a polypeptide having a combination of two heavy and two light chains whether or not it possesses any relevant specific immunoreactivity.

In one embodiment, the binding molecules of the invention comprise at least one scFv (e.g. 2, 3, or 4 scFvs, e.g., stabilized scFvs) linked to the C-terminus of an antibody heavy chain. In another embodiment, the binding molecules of the invention comprise at least one scFv (e.g. 2, 3, or 4 scFvs, e.g., stabilized scFvs) linked to the N-terminus of an antibody heavy chain. In another embodiment, the binding molecules of the invention comprise at least one scFv (e.g. 2, 3, or 4 scFvs or stabilized scFvs) linked to the N-terminus of an antibody light chain. In another embodiment, the multispecific binding molecules of the invention comprise at least one scFv (e.g., 2, 3, or 4 scFvs or stabilized scFvs) linked to the N-terminus of the antibody heavy chain or light chain and at least one scFv (e.g., 2, 3, or 4 scFvs or stabilized scFvs) linked to the C-terminus of the heavy chain.

iii. Single Domain CD23 Binding Molecules

In certain embodiments, a CD23 binding molecule is or comprises a single domain binding molecule (e.g. a single domain antibody), also known as a nanobody. Exemplary single domain molecules include an isolated heavy chain variable domain (V_{H}) of an antibody, i.e., a heavy chain variable domain, without a light chain variable domain, and an isolated light chain variable domain (V_{L}) of an antibody, i.e., a light chain variable domain, without a heavy chain variable domain. Exemplary single-domain antibodies employed in the binding molecules of the invention include, for example, the Camelid heavy chain variable domain (about 118 to 136 amino acid residues) as described in Hamers-Casterman, et al., Nature 363:446-448 (1993), and Dumoulin, et al., Protein Science 11:500-515 (2002). Multimers of single-domain antibodies are also within the scope of the invention. Other single domain antibodies include shark antibodies (e.g., shark Ig-NARs). Shark Ig-NARs comprise a homodimer of one variable domain (V-NAR) and five C-like constant domains (C-NAR), wherein diversity is concentrated in an elongated CDR3 region varying from 5 to 23 residues in length In camelid species (e.g., llamas), the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Methods for making single domain binding molecules are described in U.S. Pat. Nos. 6,005,079 and 6,765,087, both of which are incorporated herein by reference.

iv. CD23 Minibodies

In certain embodiments, the binding molecules of the invention are minibodies or comprise minibodies. Minibodies can be made using methods described in the art (see e.g., U.S. Pat. No. 5,837,821 or WO 94/09817A1). In certain embodiments, a minibody is a binding molecule that comprises only 2 complementarity determining regions (CDRs) of a naturally or non-naturally (e.g., mutagenized) occurring heavy chain variable domain or light chain variable domain, or combination thereof. An example of such a minibody is described by Pessi et al., Nature 362:367-369 (1993). Another exemplary minibody comprises a scFv molecule that is linked or fused to a CH3 domain or a complete Fc region. Multimers of minibodies are also within the scope of the invention.

In certain embodiments, the binding molecules of the invention are multivalent minibodies having at least two scFv with the same or different binding specificity. In preferred embodiments, at least one of the scFv molecules is stabilized. An exemplary minibody construct comprises a CH3 domain fused at its N-terminus to a connecting peptide which is fused at its N-terminus to a VH domain which is fused via its N-terminus to a (Gly₄Ser)n flexible linker which is fused at its N-terminus to a VL domain. In certain embodiments, multivalent minibodies may be bivalent, trivalent (e.g., triabodies), bispecific (e.g., diabodies), or tetravalent (e.g., tetrabodies).

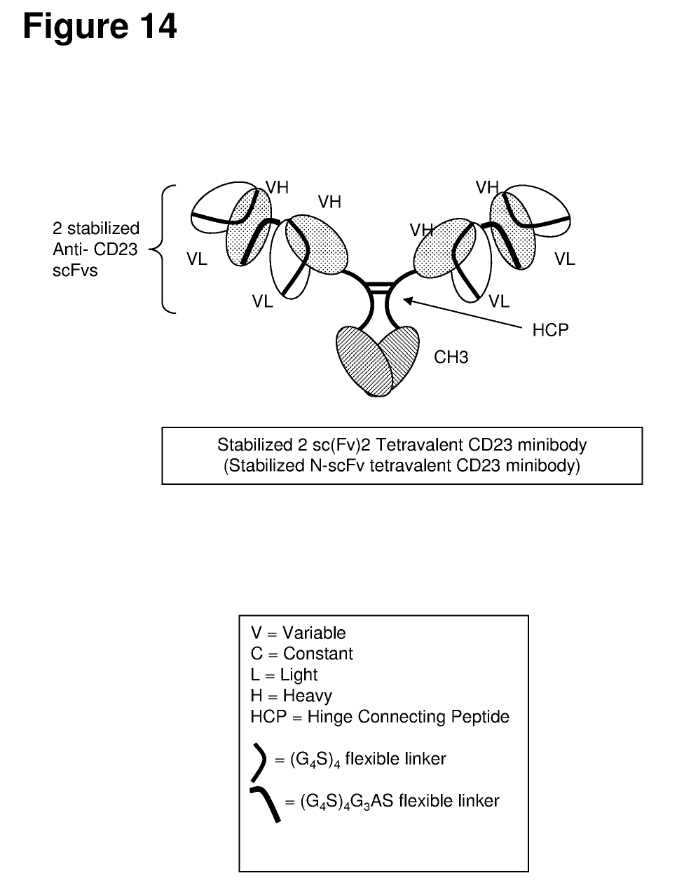

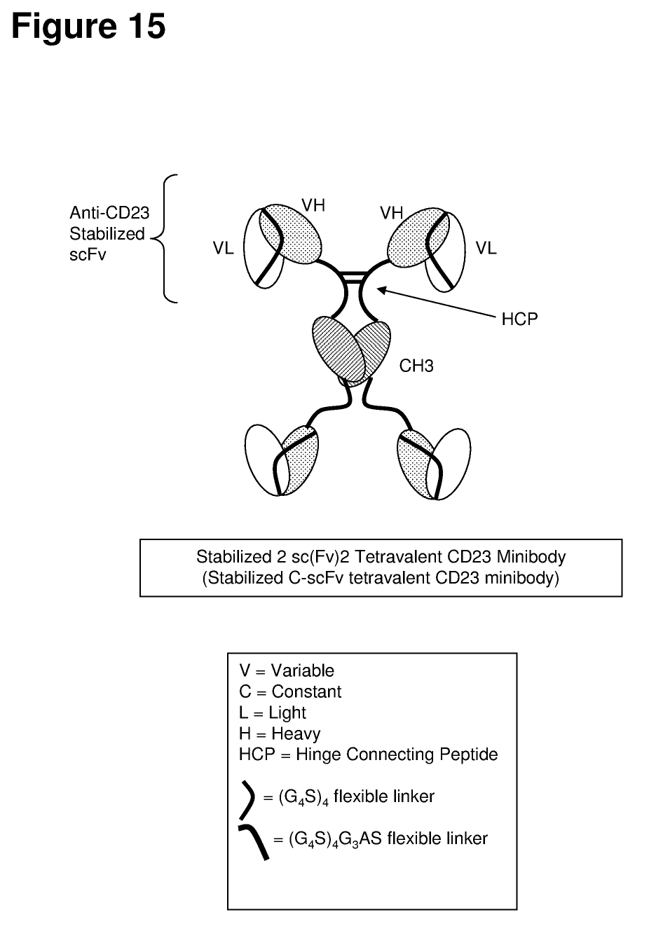

In another embodiment, the CD23 binding molecules of the invention are scFv tetravalent CD23 minibodies, with each heavy chain portion of the scFv tetravalent minibody comprises at least two scFv fragments with the same of different binding specificities. In preferred embodiments at least one of the scFv molecules is stabilized. Said second scFv fragment may be linked to the N-terminus of the first scFv fragment (e.g. N_{H} scFv tetravalent minibodies or N_{L} scFv tetravalent minibodies). Alternatively, the second scFv fragment may be linked to the C-terminus of said heavy chain portion containing said first scFv fragment (e.g. C-scFv tetravalent minibodies). In certain embodiment, where the first and second scFv fragments of a first heavy chain portion of a tetravalent minibody bind the same target CD23 molecule, at least one of the first and second scFv fragments of the second heavy chain portion of the tetravalent minibody may bind the same or different CD23 target molecule. In other embodiments, all of the scFv fragments of the tetravalent minibody bind a different CD23 target molecule. Exemplary CD23 minibodies are depicted in FIGS. 14 and 15.

v. Non-Immunoglobulin CD23 Binding Molecules

In certain embodiments, the CD23 binding molecules of the invention are non-immunoglobulin binding molecules or comprise one or more binding moieties derived from a non-immunoglobulin binding molecule. As used herein, the term “non-immunoglobulin binding molecules” are binding molecules whose binding sites comprise a portion (e.g., a scaffold or framework) which are derived from a polypeptide other than an immunoglobulin, but which may be engineered (e.g., mutagenized) to confer a desired binding specificity.

Non-immunoglobulin binding molecules can comprise binding site portions that are derived from a member of the immunoglobulin superfamily that is not an immunoglobulin (e.g. a T-cell receptor or a cell-adhesion protein (e.g., CTLA-4, N-CAM, telokin)). Such binding molecules comprise a binding site portion which retains the conformation of an immunoglobulin fold and is capable of specifically binding an IGF1-R epitope. In other embodiments, non-immunoglobulin binding molecules of the invention also comprise a binding site with a protein topology that is not based on the immunoglobulin fold (e.g. such as ankyrin repeat proteins or fibronectins) but which nonetheless are capable of specifically binding to a target (e.g. a CD23 epitope).

Non-immunoglobulin binding molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics, e.g. specific binding to a CD23 epitope described supra. For example, selection can be achieved by art-recognized methods such as phage display, yeast display, or ribosome display.

In one embodiment, a binding molecule of the invention comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. Methods for making fibronectin binding polypeptides are described, for example, in WO 01/64942 and in U.S. Pat. Nos. 6,673,901, 6,703,199, 7,078,490, and 7,119,171, which are incorporated herein by reference.

In another embodiment, a binding molecule of the invention comprises a binding site from an affibody. Affibodies are derived from the immunoglobulin binding domains of staphylococcal Protein A (SPA) (see e.g., Nord et al., Nat. Biotechnol., 15: 772-777 (1997)). Affibody binding sites employed in the invention may be synthesized by mutagenizing an SPA-related protein (e.g., Protein Z) derived from a domain of SPA (e.g., domain B) and selecting for mutant SPA-related polypeptides having binding affinity for a CD23 epitope. Other methods for making affibody binding sites are described in U.S. Pat. Nos. 6,740,734 and 6,602,977 and in WO 00/63243, each of which is incorporated herein by reference.

In another embodiment, a binding molecule of the invention comprises a binding site from an anticalin. Anticalins (also known as lipocalins) are members of a diverse β-barrel protein family whose function is to bind target molecules in their barrel/loop region. Lipocalin binding sites may be engineered to bind a CD23 epitope by randomizing loop sequences connecting the strands of the barrel (see e.g., Schlehuber et al., Drug Discov. Today, 10: 23-33 (2005); Beste et al., PNAS, 96: 1898-1903 (1999). Anticalin binding sites employed in the binding molecules of the invention may be obtainable starting from polypeptides of the lipocalin family which are mutated in four segments that correspond to the sequence positions of the linear polypeptide sequence comprising amino acid positions 28 to 45, 58 to 69, 86 to 99 and 114 to 129 of the Bilin-binding protein (BBP) of Pieris brassica. Other methods for making anticalin binding sites are described in WO99/16873 and WO 05/019254, each of which is incorporated herein by reference.

In another embodiment, a binding molecule of the invention comprises a binding site from a cysteine-rich polypeptide. Cysteine-rich domains employed in the practice of the present invention typically do not form an α-helix, a β sheet, or a β-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g., Sortilin-related receptor, LDL-receptor, VLDLR, LRP1, LRP2, and ApoER2). Methods for making A domain proteins of a desired binding specificity are disclosed, for example, in WO 02/088171 and WO 04/044011, each of which is incorporated herein by reference.

In other embodiments, a binding molecule of the invention comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)). All so far determined tertiary structures of ankyrin repeat units share a characteristic composed of a β-hairpin followed by two antiparallel α-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Other non-immunoglobulin binding sites which may be employed in binding molecules of the invention include binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al, J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, and other such domains known to those of ordinary skill in the art, as well as derivatives and/or variants thereof.

vi. CD23 Binding Molecule Fragments

In certain embodiments, a CD23 binding molecule of the invention comprises or consists of a binding molecule fragment. Unless it is specifically noted, as used herein a “fragment” in reference to a binding molecule refers to an antigen-binding fragment, i.e., a portion of the binding which specifically binds to the antigen. In one embodiment, a binding molecule of the invention is an antibody fragment or comprises such a fragment. Antibody fragments that recognize specific epitopes may be generated by known techniques. For example, Fab and F(ab′)₂ fragments may be produced recombinantly or by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

In certain embodiments, CD23 binding molecule fragments of the invention may be conjugated together. Binding molecules of the invention include conjugated Fab2 or Fab3 molecules. For example, a binding molecule fragment may comprise chemically conjugated multimers (e.g. dimers, trimers, or tetramers) of Fab or scFv molecules of the same or different binding specificities. In preferred embodiments, the Fav and scFv molecule have the same specificity.

vii. Multispecific CD23 Binding Molecules

CD23 binding molecules of the invention may comprise at least two binding sites, wherein at least one of the binding sites is derived from or comprises a CD23 binding site from one of the CD23 binding molecules described herein. In certain embodiments, at least one binding site of a multispecific binding molecule of the invention is an antigen binding region of an antibody or an antigen binding fragment thereof (e.g. an antibody or antigen binding fragment described supra).

In certain embodiments, a multispecific binding molecule of the invention is bispecific. Bispecific CD23 binding molecules may be bivalent or of a higher valency (e.g., trivalent, tetravalent, hexavalent, and the like). Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

viii. CD23 Diabodies

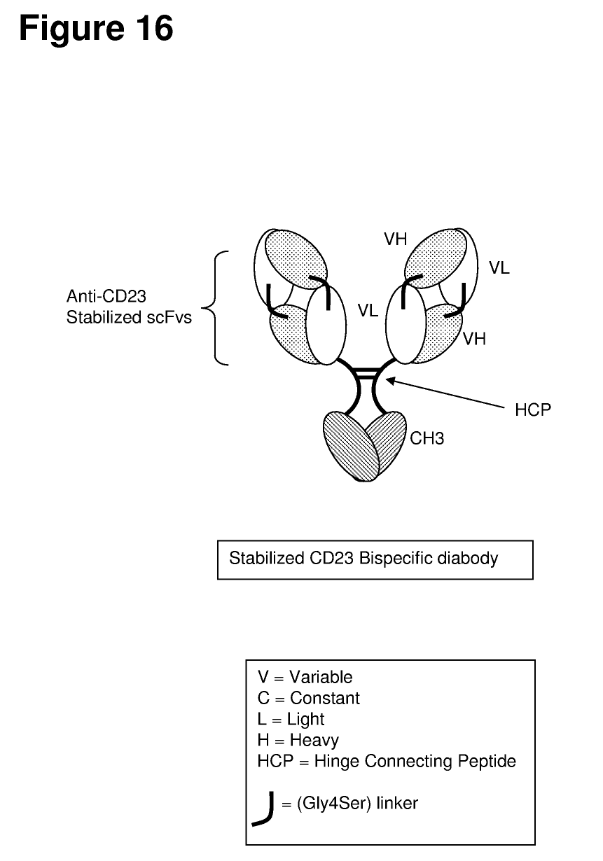

In other embodiments, the CD23 binding molecules of the invention consist or comprise of diabodies. In one embodiment, each arm of the diabody comprises tandem scFv fragments. In preferred embodiments, at least one of the scFv fragments is stabilized (e.g., stabilized scFv molecules described supra). In one embodiment, a bispecific diabody may comprise a first arm with a CD23 binding specificity and a second arm with a second binding specificity (e.g., a different CD23 binding specificity). In certain embodiments, a diabody can be directly fused to complete Fc region or an Fc portion (e.g. a CH3 domain). An exemplary CD23 diabody is depicted in FIG. 16.

ix. scFv2 Tetravalent CD23 Antibodies

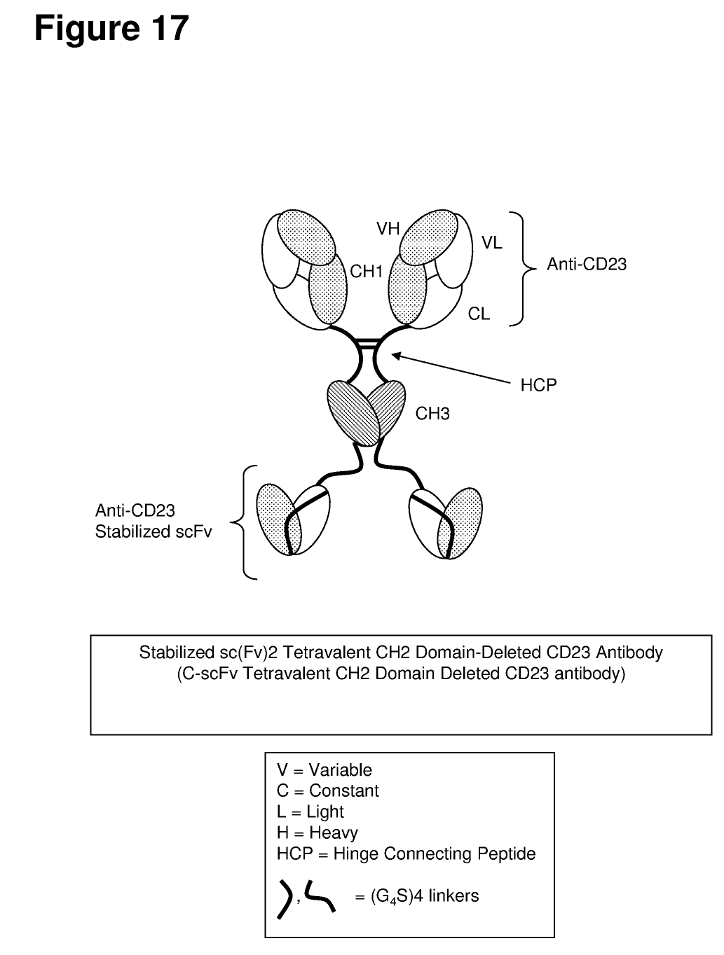

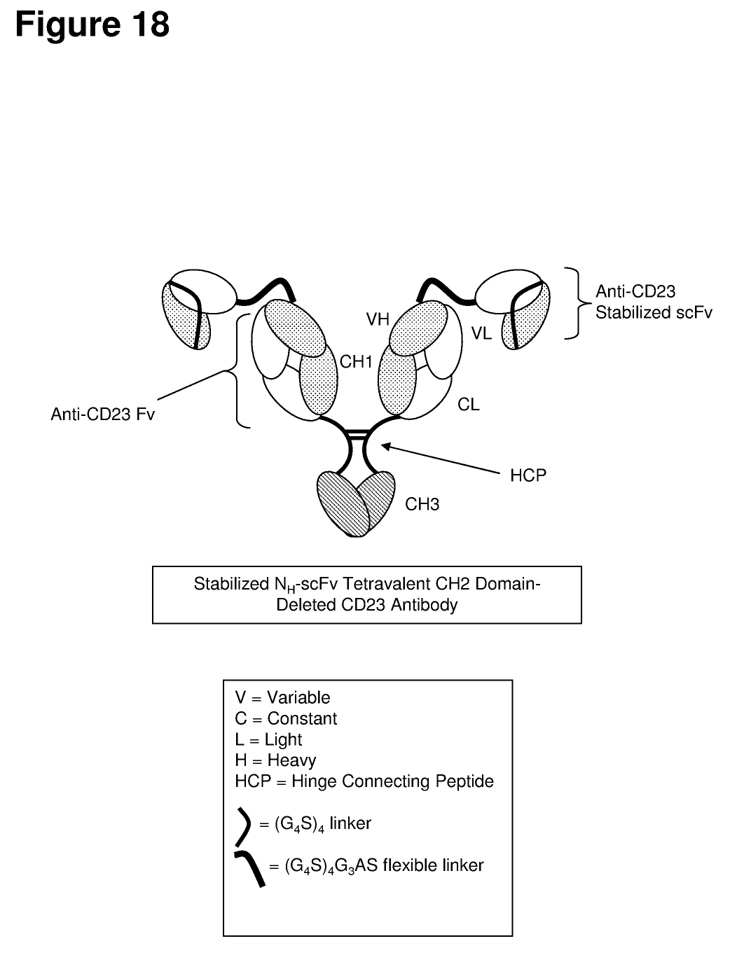

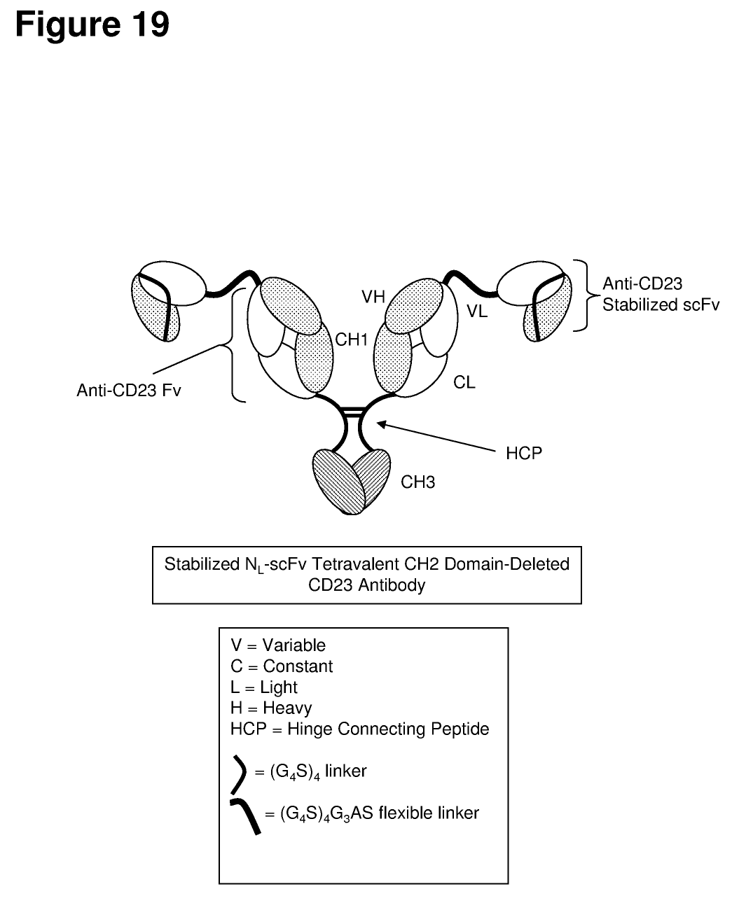

In other embodiments, the CD23 binding molecules of the invention are scFv2 tetravalent antibodies with each heavy chain portion or light chain portion of the scFv2 tetravalent antibody comprises an scFv molecule. In preferred embodiments, at least one of the scFv molecules is stabilized. The scFv fragments may be linked to the N-termini of a variable region of the heavy chain portions (e.g. bispecific N_{H} scFv2 tetravalent antibodies) or the light chain portions (e.g., bispecific N_{L} scFv2 tetravalent antibodies). Alternatively, the scFv fragments may be linked to the C-termini of the heavy chain portions of the scFv2 tetravalent antibody. In certain preferred embodiments, the heavy chain portion comprises or consists of a complete antibody heavy chain and the light chain portion comprises or consists of a complete antibody light chain (see, e.g., FIG. 2A). In other embodiments, the heavy or light chain portion is a domain deleted antibody heavy or light chain (e.g., a VL domain-deleted antibody light chain, a VH domain-deleted antibody heavy chain, or a CH2 domain-deleted antibody heavy chain, see FIGS. 17-19). Each heavy chain portion of the scFv2 tetravalent antibody may have variable regions and scFv fragments that bind the same or different target CD23 molecule or epitope. Where the scFv fragment and variable region of a first heavy chain portion of a bispecific scFc2 tetravalent antibody bind the same target molecule or epitope, at least one of the first and second scFv fragments of the second heavy chain portion of the bispecific tetravalent minibody binds a different target molecule or epitope.

x. scFv-Fc Fusions

In certain embodiments, the CD23 binding molecules of the invention are fusions of scFv molecules with the Fc region of an antibody. For example, one or more scFv molecules may be directly fused to one or both N-termini of an Fc fragment (see FIG. 2C). Additionally or alternatively, one or more scFv molecules may be directly fused to one or both C-termini of an Fc fragment. In other embodiments, one or more scFv molecules may be fused to one or both N- or C-termini of an Fc fragment via an intervening CH1 and/or CL domain (see FIG. 2B). In preferred embodiments, at least 4 scFv molecules are fused to said Fc region.

xi. Tandem Variable Domain CD23 Binding Molecules

In other embodiments, the binding molecule of the invention may comprise a binding molecule comprising tandem antigen binding sites. For example, a variable domain may comprise an antibody heavy chain that is engineered to include at least two (e.g., two, three, four, or more) variable heavy domains (VH domains) that are directly fused or linked in series, and an antibody light chain that is engineered to include at least two (e.g., two, three, four, or more) variable light domains (VL domains) that are directly fused or linked in series. The VH domains interact with corresponding VL domains to form a series of antigen binding sites wherein at least one (and preferably two or more) of the binding sites bind the same or different CD23 molecules. Tandem variable domain binding molecules may comprise two or more of heavy or light chains and are of higher order valency (e.g., tetravalent). Methods for making tandem variable domain binding molecules are known in the art, see e.g. WO 2007/024715.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

### IV. Modified CD23 Binding Molecules

In certain embodiments, CD23 binding molecules of the invention may comprise one or more modifications. Modified forms of CD23 binding molecules of the invention can be made from whole precursor or parent antibodies using techniques known in the art.

In certain embodiments, modified CD23 binding molecules of the present invention are polypeptides which have been altered so as to exhibit additional features not found on the native polypeptide. In one embodiment, one or more residues of the binding molecule may be chemically derivatized by reaction of a functional side group. In one embodiment, a binding molecule may be modified to include one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, a CD23 binding molecule of the invention comprises a synthetic constant region wherein one or more domains are partially or entirely deleted (“domain-deleted binding molecules”). In certain embodiments compatible modified binding molecules will comprise domain deleted constructs or variants wherein the entire CH2 domain has been removed (ΔCH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody. Domain deleted constructs can be derived using a vector encoding an IgG₁ human constant domain (see, e.g., WO 02/060955A2 and WO02/096948A2). This vector is engineered to delete the CH2 domain and provide a synthetic vector expressing a domain deleted IgG₁ constant region.

In one embodiment, a CD23 binding molecule of the invention comprises an immunoglobulin heavy chain having deletion or substitution of a few or even a single amino acid as long as it permits association between the monomeric subunits. For example, the mutation of a single amino acid in selected areas of the CH2 domain may be enough to substantially reduce Fc binding and thereby increase tumor localization. Similarly, it may be desirable to simply delete that part of one or more constant region domains that control the effector function (e.g. complement binding) to be modulated. Such partial deletions of the constant regions may improve selected characteristics of the antibody (serum half-life) while leaving other desirable functions associated with the subject constant region domain intact. Moreover, as alluded to above, the constant regions of the binding molecule may be synthetic through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified binding molecule. Yet other embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides binding molecule that comprise, consist essentially of, or consist of, variants (including derivatives) of binding moieties (e.g., the VH regions and/or VL regions of an antibody molecule) described herein, which binding moieties or fragments thereof immunospecifically bind to a CD23 polypeptide or fragment or variant thereof. Standard techniques known to those of skill in the art can be used to introduce mutations in the nucleotide sequence encoding a CD23 binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VH-CDR1, VH-CDR2, VH-CDR3, VL region, VL-CDR1, VL-CDR2, or VL-CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a CD23 polypeptide).

For example, it is possible to introduce mutations only in framework regions or only in CDR regions of a binding molecule of the invention (e.g., an antibody molecule). Introduced mutations may be silent or neutral missense mutations, i.e., have no, or little, effect on the ability to bind antigen, indeed some such mutations do not alter the amino acid sequence whatsoever. These types of mutations may be useful to optimize codon usage, or improve a hybridoma's antibody production. Codon-optimized coding regions encoding CD23 binding molecules of the present invention are disclosed elsewhere herein. Alternatively, non-neutral missense mutations may alter a binding molecule's ability to bind antigen. For example, in an antibody the location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a CD23 molecule) can be determined using techniques described herein or by routinely modifying techniques known in the art.

(i) Covalent Attachment

CD23 binding molecules of the invention may be modified, e.g., by the covalent attachment of a molecule to the binding molecule such that covalent attachment does not prevent the binding molecule from specifically binding to its cognate epitope. For example, but not by way of limitation, the binding molecules of the invention may be modified by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

As discussed in more detail elsewhere herein, binding molecules of the invention may further be recombinantly fused to a heterologous polypeptide at the N- or C-terminus or chemically conjugated (including covalent and non-covalent conjugations) to polypeptides or other compositions. For example, CD23-specific CD23 binding molecules may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

CD23 binding molecules may be fused to heterologous polypeptides to increase the in vivo half life or for use in immunoassays using methods known in the art. For example, in one embodiment, PEG can be conjugated to the CD23 binding molecules of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, CD23 binding molecules of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

CD23 binding molecules of the present invention may be used in non-conjugated form or may be conjugated to at least one of a variety of molecules, e.g., to improve the therapeutic properties of the molecule, to facilitate target detection, or for imaging or therapy of the patient. CD23 binding molecules of the invention can be labeled or conjugated either before or after purification, when purification is performed. In particular, CD23 binding molecules of the invention may be conjugated to therapeutic agents, prodrugs, peptides, proteins, enzymes, viruses, lipids, biological response modifiers, pharmaceutical agents, or PEG.

The present invention further encompasses CD23 binding molecules of the invention conjugated to a diagnostic or therapeutic agent. The CD23 binding molecules can be used diagnostically to, for example, monitor the development or progression of a immune cell disorder (e.g., CLL) as part of a clinical testing procedure to, e.g., determine the efficacy of a given treatment and/or prevention regimen. Detection can be facilitated by coupling the CD23 binding molecule to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

CD23 binding molecules for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG.

In another embodiment, a binding molecule for use in the diagnostic and treatment methods disclosed herein can be conjugated to a molecule that decreases tumor cell growth. In other embodiments, the disclosed compositions may comprise binding molecules coupled to drugs or prodrugs. Still other embodiments of the present invention comprise the use of binding molecules conjugated to specific biotoxins or their cytotoxic fragments such as ricin, gelonin, Pseudomonas exotoxin or diphtheria toxin. The selection of which conjugated or unconjugated binding molecule to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy cells in lymphomas or leukemias in animal models, and in some cases in humans. Exemplary radioisotopes include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. The radionuclides act by producing ionizing radiation which causes multiple strand breaks in nuclear DNA, leading to cell death. The isotopes used to produce therapeutic conjugates typically produce high energy α- or β-particles which have a short path length. Such radionuclides kill cells to which they are in close proximity, for example neoplastic cells to which the conjugate has attached or has entered. They have little or no effect on non-localized cells. Radionuclides are essentially non-immunogenic.

(ii) Reducing Immunogenicity

In certain embodiments, CD23 binding molecules of the invention or portions thereof are modified to reduce their immunogenicity using art-recognized techniques. For example, binding molecules or portions thereof can be humanized, primatized, or deimmunized. In one embodiment, chimeric binding molecules can be made or binding molecules may comprise at least a portion of a chimeric antibody molecule. In such case a non-human CD23 binding molecule, typically a murine or primate binding molecule, that retains or substantially retains the antigen-binding properties of the parent binding molecule, but which is less immunogenic in humans is constructed. This may be achieved by various methods, including (a) grafting the entire non-human variable domains onto human constant regions to generate chimeric binding molecule; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

In one embodiment, a binding molecule (e.g., an antibody) of the invention or portion thereof may be chimeric. A chimeric binding molecule is a binding molecule in which different portions of the binding molecule are derived from different animal species, such as antibodies having a variable region derived from a murine monoclonal antibody and a human immunoglobulin constant region. Methods for producing chimeric binding molecules are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) may be employed for the synthesis of said molecules. For example, a genetic sequence encoding a binding specificity of a mouse CD23 antibody molecule may be fused together with a sequence from a human antibody molecule of appropriate biological activity can be used. As used herein, a chimeric binding molecule is a molecule in which different portions are derived from different animal species, such as those having a variable region derived from a murine monoclonal antibody and a human immunoglobulin constant region, e.g., humanized antibodies.

In another embodiment, a binding molecule of the invention or portion thereof is primatized. Methods for primatizing antibodies are disclosed by Newman, Biotechnology 10: 1455-1460 (1992). Specifically, this technique results in the generation of antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, a binding molecule (e.g., an antibody) of the invention or portion thereof is humanized. Humanized binding molecules are binding molecules having a binding specificity from non-human species antibody that binds the desired antigen having one or more complementarity determining regions (CDRs) from the non-human species antibody and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

De-immunization can also be used to decrease the immunogenicity of a binding molecule. As used herein, the term “de-immunization” includes alteration of a binding molecule to modify T cell epitopes (see, e.g., WO9852976A1, WO0034317A2). For example, VH and VL sequences from the starting antibody may be analyzed and a human T cell epitope “map” may be generated from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., CD23-specific antibodies or immunospecific fragments thereof for use in the diagnostic and treatment methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

(iii) Effector Functions and Fc Modifications

CD23 binding molecules of the invention may comprise a constant region (e.g. constant regions derived from an IgG antibody, e.g. an IgG1 antibody) which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In preferred embodiments, the binding molecules of the invention bind to an Fcγ receptor. In alternative embodiments, CD23 binding molecules of the invention may comprise a constant region which is devoid of one or more effector functions (e.g., ADCC activity) and/or is unable to bind Fcγ receptor.

Certain embodiments of the invention include CD23 binding molecules in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, or increased serum half-life when compared with a whole, unaltered antibody of approximately the same immunogenicity. For example, certain binding molecules for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain other embodiments, a CD23 binding molecule comprises constant regions derived from different antibody isotypes (e.g., constant regions from two or more of a human IgG1, IgG2, IgG3, or IgG4). In other embodiments, a CD23 binding molecule comprises a chimeric hinge (i.e., a hinge comprising hinge portions derived from hinge domains of different antibody isotypes, e.g., an upper hinge domain from an IgG4 molecule and an IgG1 middle hinge domain). In one embodiment, a CD23 binding molecule comprises an Fc region or portion thereof from a human IgG4 molecule and a Ser228Pro mutation (EU numbering) in the core hinge region of the molecule.

In certain CD23 binding molecules, the Fc portion may be mutated to increase or decrease effector function using techniques known in the art. For example, the deletion or inactivation (through point mutations or other means) of a constant region domain may reduce Fc receptor binding of the circulating modified binding molecule thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or flexibility. The resulting physiological profile, bioavailability and other biochemical effects of the modifications, such as tumor localization, biodistribution and serum half-life, may easily be measured and quantified using well know immunological techniques without undue experimentation.

In certain embodiments, an Fc domain employed in a binding polypeptide of the invention is an Fc variant. As used herein, the term “Fc variant” refers to an Fc domain having at least one amino acid substitution relative to the wild-type Fc domain from which said Fc domain is derived. For example, wherein the Fc domain is derived from a human IgG1 antibody, the Fc variant of said human IgG1 Fc domain comprises at least one amino acid substitution relative to said Fc domain.

The amino acid substitution(s) of an Fc variant may be located at any position (i.e., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The binding polypeptides of the invention may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated by reference herein. In one exemplary embodiment, a binding molecule of the invention may comprise an Fc variant comprising an amino acid substitution at EU position 268 (e.g., H268D or H268E). In another exemplary embodiment, a binding molecule of the invention may comprise an amino acid substitution at EU position 239 (e.g., S239D or S239E) and/or EU position 332 (e.g., I332D or I332Q).

In preferred embodiments, binding polypeptide may comprise an Fc variant comprising an amino acid substitution an EU amino acid position that is within the “15 Angstrom Contact Zone” of the Fc domain. The 15 Angstrom Zone includes residues located at EU positions 243 to 261, 275 to 280, 282-293, 302 to 319, 336 to 348, 367, 369, 372 to 389, 391, 393, 408, and 424-440 of the Fc region.

In certain embodiments, a binding polypeptide of the invention may comprise an Fc variant comprising an amino acid substitution which alters the antigen-independent effector functions of the antibody, in particular the circulating half-life of the antibody. Such binding polypeptides exhibit either increased or decreased binding to FcRn when compared to binding polypeptides lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered polypeptide is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the altered polypeptides of the invention exhibit reduced transport across the epithelium of kidney glomeruli from the vasculature. In another embodiment, the altered polypeptides of the invention exhibit reduced transport across the blood brain barrier (BBB) from the brain, into the vascular space. In one embodiment, a binding polypeptide with altered FcRn binding comprises an Fc domain having one or more amino acid substitutions within the “FcRn binding loop” of an Fc domain. The FcRn binding loop is comprised of amino acid residues 280-299 (according to EU numbering). In other embodiment, a binding polypeptide of the invention having altered FcRn binding affinity comprises an Fc domain having one or more amino acid substitutions within the 15 Å FcRn “contact zone.” As used herein, the term 15 Å FcRn “contact zone” includes residues at the following positions 243-261, 275-280, 282-293, 302-319, 336-348, 367, 369, 372-389, 391, 393, 408, 424, 425-440 (EU numbering). In preferred embodiments, a binding polypeptide of the invention having altered FcRn binding affinity comprises an Fc domain having one or more amino acid substitutions at any one of the following positions: 256, 277-281, 283-288, 303-309, 313, 338, 342, 376, 381, 384, 385, 387, 434, and 438. Exemplary amino acid substitutions which altered FcRn binding activity are disclosed in International PCT Publication No. WO05/047327 which is incorporated by reference herein. In certain exemplary embodiments, the binding molecules of the invention comprise an Fc domain having one or more of the following substitutions: V284E, H285E, N286D, K290E and S304D (EU numbering).

In other embodiments, certain binding molecules for use in the diagnostic and treatment methods described herein have a constant region, e.g., an IgG1 or IgG4 heavy chain constant region, which is altered to reduce or eliminate glycosylation. For example, a binding polypeptide of the invention may also comprise an Fc variant comprising an amino acid substitution which alters the glycosylation of the binding polypeptide. For example, said Fc variant may have reduced glycosylation (e.g., N- or O-linked glycosylation). In exemplary embodiments, the Fc variant comprises reduced glycosylation of the N-linked glycan normally found at amino acid position 297 (EU numbering). In another embodiment, the binding polypeptide has an amino acid substitution near or within a glycosylation motif, for example, an N-linked glycosylation motif that contains the amino acid sequence NXT or NXS. In a particular embodiment, the binding polypeptide comprises an Fc variant with an amino acid substitution at amino acid position 228 or 299 (EU numbering). In more particular embodiments, the binding molecule comprises an IgG1 or IgG4 constant region comprising an S228P and a T299A mutation (EU numbering).

Exemplary amino acid substitutions which confer reduce or altered glycosylation are disclosed in International PCT Publication No. WO05/018572, which is incorporated by reference herein. In preferred embodiments, the binding molecules of the invention are modified to eliminate glycosylation. Such binding molecules may be referred to as “agly” binding molecules (e.g. “agly” antibodies). While not being bound by theory, it is believed that “agly” binding molecules may have an improved safety and stability profile in vivo. Exemplary agly binding molecules comprise an aglycosylated Fc region of an IgG4 antibody (“IgG4.P”) which is devoid of Fc-effector function thereby eliminating the potential for Fc mediated toxicity to the normal vital organs that express CD23. In yet other embodiments, binding molecules of the invention comprise an altered glycan. For example, the binding molecule may have a reduced number of fucose residues on an N-glycan at Asn297 of the Fc region, i.e., is afucosylated. In another embodiment, the binding molecule may have an altered number of sialic acid residues on the N-glycan at Asn297 of the Fc region.

### V. Expression of CD23 Binding Molecules

Following manipulation of the isolated genetic material to provide polypeptides of the invention as set forth above, the genes are typically inserted in an expression vector for introduction into host cells that may be used to produce the desired quantity of polypeptide that, in turn, provides the claimed binding molecules.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals. In particularly preferred embodiments the cloned variable region genes are inserted into an expression vector along with the heavy and light chain constant region genes (preferably human) synthetic as discussed above. Preferably, this is effected using a proprietary expression vector of IDEC, Inc., referred to as NEOSPLA (U.S. Pat. No. 6,159,730). This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples below, this vector has been found to result in very high level expression of antibodies upon incorporation of variable and constant region genes, transfection in CHO cells, followed by selection in G418 containing medium and methotrexate amplification. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the polypeptides of the invention of the instant invention may be expressed using polycistronic constructs such as those disclosed in copending U.S. provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of polypeptides of the invention in eukaryotic host cells. Compatible IRES sequences are disclosed in U.S. Pat. No. 6,193,980 which is also incorporated herein. Those skilled in the art will appreciate that such expression systems may be used to effectively produce the full range of polypeptides disclosed in the instant application.

More generally, once the vector or DNA sequence encoding a monomeric subunit of the binding molecule (e.g. a modified antibody) has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for protein expression (e.g., of multivalent binding molecules) is of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte), 293 (human kidney). In one embodiment, the cell line provides for altered glycosylation, e.g., afucosylation, of the binding molecule expressed therefrom (e.g., PER.C6® (Crucell) or FUT8-knock-out CHO cell lines (Potelligent® Cells) (Biowa, Princeton, N.J.)). In one embodiment NS0 cells may be used. CHO cells are particularly preferred. Host cell lines are typically available from commercial services, the American Tissue Culture Collection or from published literature.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding the polypeptide of the invention can also be expressed non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules. Where tetravalent forms of antibodies are desired, the subunits will then self-assemble into tetravalent antibodies (WO02/096948A2).

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### VII. Pharmaceutical Formulations and Methods of Administration of Binding Molecules

Methods of preparing and administering binding molecules of the invention to a subject are well known to or are readily determined by those skilled in the art. The route of administration of the binding molecules of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

Effective doses of the stabilized binding molecules of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with a binding molecule of the invention, the dosage may range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention.

Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered may fall within the ranges indicated.

Binding molecules of the invention can be administered on multiple occasions. Intervals between single dosages can be, e.g., daily, weekly, monthly or yearly. Intervals can also be irregular as indicated by measuring blood levels of polypeptide or target molecule in the patient. In some methods, dosage is adjusted to achieve a certain plasma binding molecule or toxin concentration, e.g., 1-1000 μg/ml or 25-300 μg/ml. Alternatively, binding molecules can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies. In one embodiment, the binding molecules of the invention can be administered in unconjugated form, In another embodiment, the polypeptides of the invention can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a polypeptide of the invention (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. Intramuscular injection or intravenous infusion are preferred for administration of a binding molecule of the invention. In some methods, particular therapeutic binding molecules are injected directly into the cranium. In some methods, binding molecules are administered as a sustained release composition or device, such as a Medipad™ device.

Agents of the invention can optionally be administered in combination with other agents that are effective in treating the disorder or condition in need of treatment (e.g., prophylactic or therapeutic). Preferred additional agents are those which are art recognized and are standardly administered for a particular disorder.

Effective single treatment dosages (i.e., therapeutically effective amounts) of ⁹⁰Y-labeled polypeptides of the invention range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric modified antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²²⁵Ac, ²¹¹A ²¹³Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not the binding molecules of the invention are used in a conjugated or unconjugated form, it will be appreciated that a major advantage of the present invention is the ability to use these polypeptides in myelosuppressed patients, especially those who are undergoing, or have undergone, adjunct therapies such as radiotherapy or chemotherapy. In other preferred embodiments, the polypeptides (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies and one or more chemotherapeutic agents. Particularly preferred embodiments of this aspect of the invention will comprise the administration of a CD23 binding molecule of the invention, together with one or more conventional CLL chemotherapeutics (e.g., Fludarabine and/or Cyclophosphamide) and, optionally, an anti-CD20 antibody (e.g., Rituximab, Ocrelizumab, or Ofatumumab).

While the binding molecules may be administered as described immediately above, it must be emphasized that, in other embodiments, conjugated and unconjugated polypeptides may be administered to otherwise healthy patients as a first line therapeutic agent. In such embodiments the polypeptides may be administered to patients having normal or average red marrow reserves and/or to patients that have not, and are not, undergoing adjunct therapies such as external beam radiation or chemotherapy.

However, as discussed above, selected embodiments of the invention comprise the administration of binding molecules to myelosuppressed patients or in combination or conjunction with one or more adjunct therapies such as radiotherapy or chemotherapy (i.e. a combined therapeutic regimen). As used herein, the administration of polypeptides in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated polypeptides could be administered intravenously followed by tumor localized external beam radiation. In yet other embodiments, the polypeptide may be administered concurrently with one or more selected chemotherapeutic agents in a single office visit. A skilled artisan (e.g. an experienced oncologist) would readily be able to discern effective combined therapeutic regimens without undue experimentation based on the selected adjunct therapy and the teachings of the instant specification.

In this regard it will be appreciated that the combination of the binding molecules (either conjugated or unconjugated) and the chemotherapeutic agent may be administered in any order and within any time frame that provides a therapeutic benefit to the patient. That is, the chemotherapeutic agent and polypeptide may be administered in any order or concurrently. Binding molecules and chemotherapeutic agents may be administered separately or may be administered in the form of one composition. In selected embodiments the polypeptides of the present invention will be administered to patients that have previously undergone chemotherapy. In yet other embodiments, the polypeptides and the chemotherapeutic treatment will be administered substantially simultaneously or concurrently. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the polypeptide will be administered within 4, 3, 2 or 1 week of any chemotherapeutic agent or treatment. In yet other embodiments the polypeptide will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppresion and one can easily measure the extent to which myelosuppresion is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated polypeptides of the present invention may be used to effectively treat patients having ANCs lower than about 2000/mm³ or platelet counts lower than about 150,000/mm³. More preferably the polypeptides of the present invention may be used to treat patients having ANCs of less than about 1500/mm³, less than about 1000/mm³ or even more preferably less than about 500/mm³. Similarly, the polypeptides of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, the disclosed polypeptides may be used to treat disorders in patients exhibiting myelosuppression regardless of the cause.

In this regard it will further be appreciated that the polypeptides of the instant invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include the three-drug combination FCR (e.g., Fludarabine, Cyclophosphamide, and anti-CD20 antibody (e.g., Rituxan™)). This combination is particularly useful in patients with relapsed CLL. Other potentially useful chemotherapeutic agents include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13 ed. 1994) and V. T. DeVita et al., (1997) and the references cited therein for standard dosing and scheduling. These therapies can be used unchanged, or altered as needed for a particular patient, in combination with one or more polypeptides of the invention as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the polypeptides of the instant invention may vary by subject or may be administered according to what is known in the art. See for example, Bruce A Chabner et al., Antineoplastic Agents, in GOODMAN & GILMAN's THE PHARMACOLOGICAL BASIS OF THERAPEUTICS 1233-1287 ((Joel G. Hardman et al., eds., 9^{th} ed. 1996).

In one embodiment, a binding molecule of the invention may be administered to a subject who has undergone, is undergoing, or will undergo a surgical procedure, e.g., to remove a primary tumor, a metastasis or precancerous growth or tissue as a preventative therapy.

In another embodiment, a binding molecule of the invention is administered in conjunction with a biologic. Biologics useful in the treatment of cancers are known in the art and a binding molecule of the invention may be administered, for example, in conjunction with such known biologics. For example, the FDA has approved the following biologics for the treatment of leukemia/lymphomas: Zevalin® (ibritumomab tiuxetan, Biogen Idec, Cambridge, Mass.; Bexxar® (tositumomab and iodine I-131 tositumomab, GlaxoSmithKline, Research Triangle Park, N.C.; a multi-step treatment involving a mouse monoclonal antibody (tositumomab) linked to a radioactive molecule (iodine I-131)); Intron® A (interferon alfa-2b, Schering Corporation, Kenilworth, N.J.; a type of interferon approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; and Ontak® (denileukin diftitox, Ligand Pharmaceuticals Inc., San Diego, Calif.; a fusion protein consisting of a fragment of diphtheria toxin genetically fused to interleukin-2).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

As previously discussed, the binding molecules of the present invention, immunoreactive fragments or recombinants thereof may be administered in a pharmaceutically effective amount for the in vivo treatment of mammalian disorders. In this regard, it will be appreciated that the disclosed binding molecules will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of a binding molecule of the invention, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the polypeptide will be preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the polypeptide.

In keeping with the scope of the present disclosure, the polypeptides of the invention may be administered to a human or other animal in accordance with the aforementioned methods of treatment in an amount sufficient to produce a therapeutic or prophylactic effect. The polypeptides of the invention can be administered to such human or other animal in a conventional dosage form prepared by combining the binding molecule of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides according to the present invention may prove to be particularly effective.

### VIII. Methods of Treating Immune Cell Disorders

The subject binding molecules are useful for reducing or eliminating immune cells (e.g. by apoptosis) bearing CD23 molecules. In certain embodiments, the binding molecules of the invention inhibit are used to prevent or treat an immune cell disorder in a mammalian subject, in particular a human. For example, the binding molecules may be used to inhibit tumor cell growth and/or prolong the survival time of a subject having a hematological malignancy (e.g. a lymphoma/leukemia) that expresses CD23 on its cell surface (e.g., CLL or Small Lymphocytic Lymphoma (SLL)). Accordingly, this invention also relates to a method of treating hematological malignancies in a human or other animal by administering to such human or animal an effective, non-toxic amount of CD23 binding molecule.

In particular embodiments, the CD23 binding molecules of the invention are useful for eliminating leukemia/lymphomas bearing CD23. In exemplary embodiments, the binding molecules of the invention useful for treating Chronic Lymphocytic Leukemia (CLL) by reducing the concentration of or eliminating CLL B-cell in the circulation of a subject suffering from CLL. In preferred embodiments, a CD23 binding molecule of the invention is administered together with standard FCR chemotherapy (Fludarabine, Cyclophosphamide, and Rituxan™).

In general, the disclosed compositions may be used prophylactically or therapeutically. For example, a neoplasm comprising a marker that allows for the targeting of the cancerous cells by the binding molecule may be detected or inhibited (e.g., killed) using a binding molecule of the invention. In a preferred embodiment, the binding molecules of the invention are used to treat chronic lymphocytic leukemia (CLL). Other hematologic malignancies that are amenable to treatment with the disclosed invention include Hodgkins and non-Hodgkins lymphoma as well as leukemias, including ALL-L3 (Burkitt's type leukemia), and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated molecules.

In yet other embodiments the CD23 binding molecules of the present invention may be used to treat other immune disorders associated with CD23 expression that include, but are not limited to, allergic disorders, inflammatory disorders, or autoimmune disorders. Exemplary immune disorders include allergic bronchopulmonary aspergillosis; Allergic rhinitis; Autoimmune hemolytic anemia; Acanthosis nigricans; Allergic contact dermatitis; Addison's disease; Atopic dermatitis; Alopecia greata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

One skilled in the art would be able, by routine experimentation, to determine what an effective, non-toxic amount of polypeptide would be for the purpose of an immune cell disorder associated with CD23. For example, a therapeutically active amount of a polypeptide may vary according to factors such as the disease stage (e.g., stage I versus stage IV), age, sex, medical complications (e.g., immunosuppressed conditions or diseases) and weight of the subject, and the ability of the binding molecule to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

For purposes of clarification “mammal” refers to any animal classified as a mammal, including humans, domestic and farm animals, and zoo, sports, or pet animals, such as dogs, horses, cats, cows, etc. Preferably, the mammal is human. “Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease.

This invention is further illustrated by the following examples which should not be construed as limiting. The contents of all references, patents and published patent applications cited throughout this application are incorporated herein by reference.

### EXAMPLES

Throughout the examples, the following materials and methods were used unless otherwise stated.

### General Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Expression Constructs

In general, unless otherwise indicated, the expression constructs for scFvs and antibody heavy chain in the following Examples included a nucleotide sequence encoding an N-terminal signal peptide having the amino acid sequence MGWSLILLFLVAVATRVLS (SEQ ID NO: 21). Expression constructs for antibody light chains included a nucleotide sequence encoding the amino acid sequence MDMRVPAQLLGLLLLWLPGARC (SEQ ID NO:22). It will be understood that these signal sequences are not part of the expressed mature protein.

### Example 1

### Preparation of a PRIMATIZED® p5E8 Tetravalent Antibody Comprising a Conventional p5E8 scFv

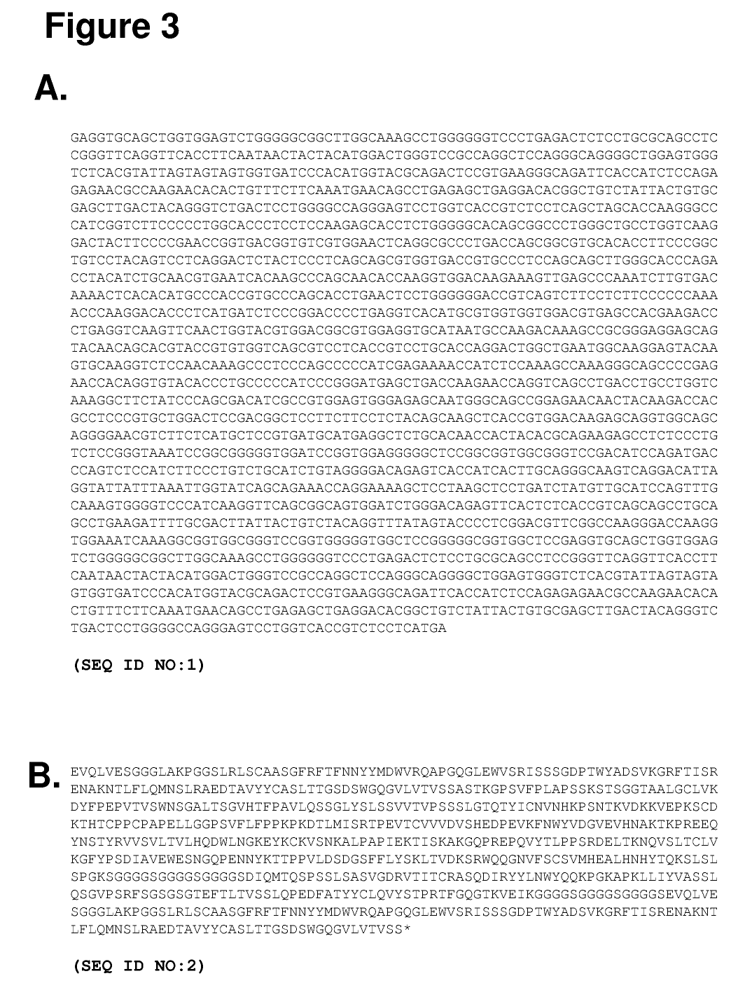

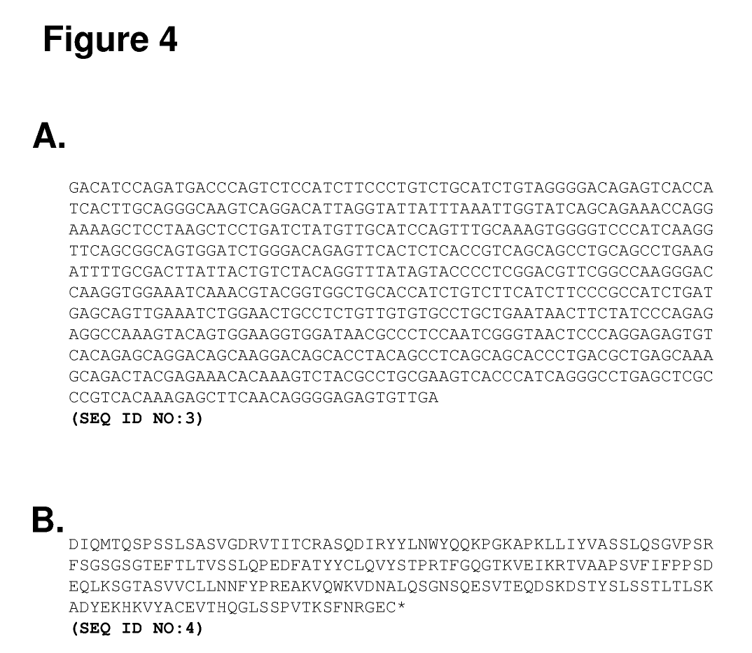

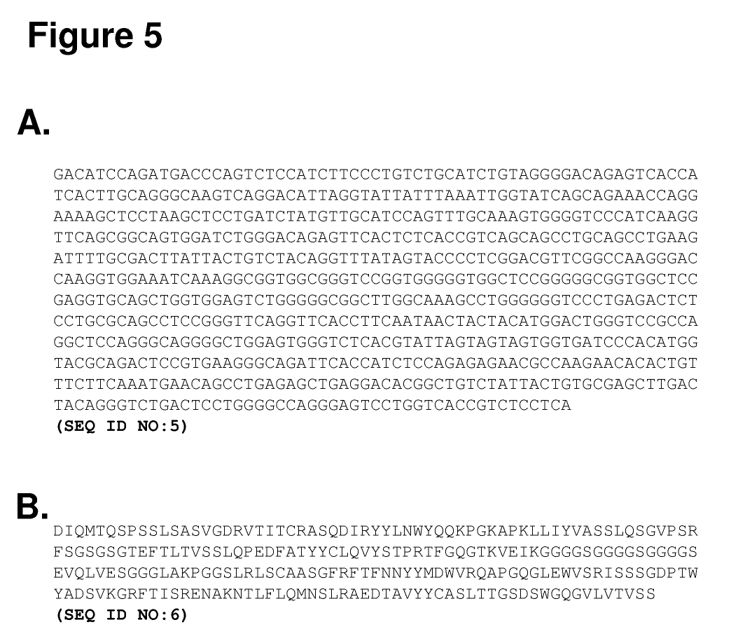

DNA and amino acid sequences for both the heavy chain PRIMATIZED® p5E8 scFv fusion protein and light chain PRIMATIZED® p5E8 are shown in FIGS. 3 and 4, respectively. The conventional PRIMATIZED® p5E8 scFv used for constructing the tetravalent antibody is comprised of p5E8 VL and VH region sequences tethered by a short linker in the VL→(Gly₄Ser)₃ linker→VH orientation. DNA and amino acid sequences of the conventional p5E8 VL/VH scFv are shown in FIGS. 5A and 5B, respectively. Correct sequences were confirmed by DNA sequence analysis. Plasmid DNA was used to transform CHO DG44 cells for stable production of antibody protein.

### Example 2

### Preparation of PRIMATIZED® p5E8 scFv and Fab Proteins

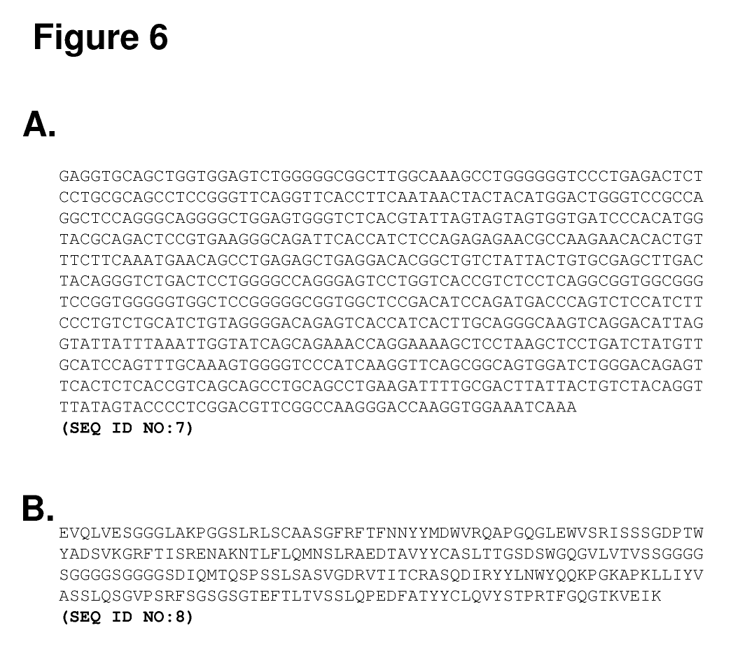

p5E8 scFvs in the orientation VL→(Gly₄Ser)₃ linker→VH (VL/VH, FIGS. 5A and 5B) and VH→(Gly₄Ser)₃ linker→VL (VH/VL, FIGS. 6A and 6B) were subcloned by PCR amplification from plasmids described in U.S. Patent Application 20050163782. Oligonucleotides used in the construction are shown in Table 1. p5E8 scFv (VL/VH) was constructed by PCR using the forward primer P5E8-VL01F which contains 29 bases encoding part of the gpIII leader sequence followed by 15 bases of sequence complementary to the p5E8 N-terminal light variable domain gene and the reverse primer, P5E8-VH01R, which contains 15 bases of sequence complementary to the p5E8 C-terminal heavy variable domain followed by a unique adjacent Sal I endonuclease site (endonuclease site is underlined). Similarly, p5E8 scFv (VH/VL) was constructed by PCR using the forward primer P5E8-VH01F which contains 29 bases encoding part of the gpIII leader sequence followed by 18 bases of sequence complementary to the p5E8 N-terminal variable heavy domain gene and the reverse primer, P5E8-VL01R, which contains 12 bases of sequence complementary to the p5E8 C-terminal variable light domain gene followed by a unique adjacent Sal I endonuclease site (endonuclease site is underlined).

Following PCR amplification, primer P5E8-Leader01 was added to both reactions for a second PCR reaction. Primer P5E8-Leader01 contains a unique Nde I endonuclease site followed by 25 bases encoding the N-terminal portion of the gpIII leader sequence, followed by 22 bases complementary to the 5′ ends of P5E8-VL01F and P5E8-VH01F and PCR amplified again. PCR products corresponding to the expected sizes were resolved by agarose gel electrophoresis, excised, and purified using the Millipore Ultrafree-DA extraction kit according to manufacturer's instructions (Millipore; Bedford, Mass.). The purified PCR products were subsequently digested with Nde I and Sal I and cloned into the Nde I/Sal I sites of a modified E. coli expression vector designed to drive recombinant protein expression under the control of an inducible ara C promoter. The expression vector contained a modification encoding a unique Nde I site overlapping the start codon of the BHA10 scFv. Individual ligation reactions were performed with each of the gel purified PCR products and the digested expression vector and a portion of each of the ligation mixtures were used to transform E. coli strain XL1-Blue. Ampicillin drug resistant colonies were screened and DNA sequence analysis confirmed the correct sequence of the final p5E8 (VH/VL) encoding pIEH162 and p5E8 (VL/VH) encoding pIEH163 constructs. DNA and amino acid sequences of p5E8 (VL/VH) scFv are shown in FIGS. 5A and 5B, respectively. DNA and amino acid sequences of p5E8 (VH/VL) scFv are shown in FIGS. 6A and 6B, respectively.

TABLE 1

Oligonucleotides for PCR amplification of a
conventional p5E8 scFvs.

| | |
|---|---|
| Primers | Sequence |

| | |
|---|---|
| P5E8-VL01F | 5′- CGCTGGTGGTGCCGTTCTATAGCCATAGTGAC |
| (SEQ ID NO:23) | ATCCAGATGACC -3′ |
| P5E8-VL01R | 5′- GTGGTCGACTTTGATTTCCAC -3′ |
| (SEQ ID NO:24) | |
| P5E8-VH01F | 5′- CGCTGGTGGTGCCGTTCTATAGCCATAGTGAG |
| (SEQ ID NO:25) | GTGCAGCTGGTGGAG -3′ |
| P5E8-VH01R | 5′- GTGGTCGACTGAGGAGACGGTGAC -3′ |
| (SEQ ID NO:26) | |
| P5E8-Leader01 | 5′- GGCATATGAAAAAACTGCTGTTCGCGATTCCG |
| (SEQ ID NO:27) | CTGGTGGTGCCGTTCTATAG -3′ |

For expression of conventional p5E8 scFvs, freshly isolated colonies of E. coli strain W3110 (ATCC, Manassas, Va. Cat. #27325) transformed with plasmids pIEH162 and pIEH163 were cultivated and either culture supernatants or periplasm extracts were prepared as described in U.S. patent application Ser. No. 11/725,970, which is incorporated by reference herein in its entirety. PRIMATIZED® p5E8 FAb was prepared by enzymatic digestion of PRIMATIZED® p5E8 IgG as previously described. Purified FAb was concentrated to between 2-11 mg/mL. Fab concentrations were determined using an ε_{280 nm}=1.5 mL mg⁻¹ cm⁻¹.

### Example 3

### Thermal Stability of Conventional p5E8 scFv Antibodies

A thermal challenge assay described in U.S. patent application Ser. No. 11/725,970 was employed as a stability screen to determine the temperature at which 50% of the p5E8 (VL/VH) and p5E8 (VH/VL) scFvs molecules retain their antigen binding activity following a thermal challenge event.

E. coli strain W3110 (ATCC, Manassas, Va. Cat. #27325) was transformed with plasmids encoding p5E8 (VL/VH) and p5E8 (VH/VL) scFvs under the control of an inducible ara C promoter. Transformants were grown overnight in expression media consisting of SB (Teknova, Half Moon Bay, Calif. Cat. #S0140) supplemented with 0.6% glycine, 0.6% Triton X100, 0.02% arabinose, and 50 μg/ml carbenicillin at 30° C. Bacteria was pelleted by centrifugation and supernatants harvested for further treatment.

After thermal challenge, the aggregated material was removed by centrifugation and soluble scFv samples remaining in the treated, cleared supernatant were assayed for binding to cognate soluble CD23 antigen by DELFIA assay. A 96-well plate (MaxiSorp, Nalge Nunc, Rochester, N.Y., Cat. #437111) was coated overnight at 4° C. with soluble CD23 antigen at 1 μg/ml in PBS, and then blocked with DELFIA assay buffer (DAB, 10 mM Tris HCl, 150 mM NaCl, 20 μM EDTA, 0.5% BSA, 0.02% Tween 20, 0.01% NaN₃, pH 7.4) for one hour with shaking at room temperature. The plate was washed 3 times with DAB without BSA (Wash buffer), and test samples diluted in DAB were added to the plates in a final volume of 100 μl. The plate was incubated for one hour with shaking at room temperature, and then washed 3 times with Wash buffer to remove unbound and functionally inactivated scFv molecules. Bound scFv was detected by addition of 100 μl per well of DAB containing 250 ng/ml of Eu-labeled anti-His₆ antibody (Perkin Elmer, Boston, Mass., Cat. #AD0109) and incubated at room temperature with shaking for one hour. The plate was washed 3 times with Wash buffer, and 100 μl of DELFIA enhancement solution (Perkin Elmer, Boston, Mass., Cat. #4001-0010) was added per well. Following incubation for 15 minutes, the plate was read using the Europium method on a Victor 2 (Perkin Elmer, Boston, Mass.). Data was analyzed using Prism 4 software (GraphPad Software, San Diego, Calif.) using a sigmoidal dose response with variable slope as the model. The values obtained for the mid-point of the thermal denaturation curves are referred to as T₅₀ values, and are not construed as being equivalent to biophysically derived Tm values.

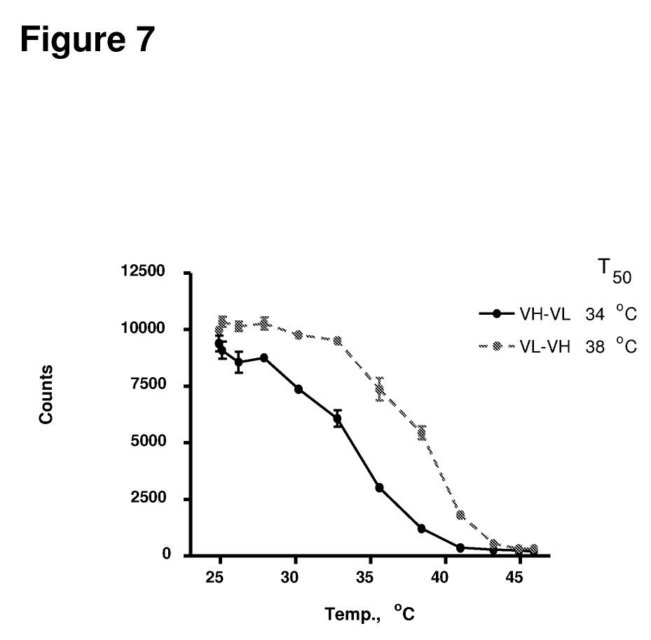

Results from this assay determined the T₅₀ value of p5E8 (VL/VH) to be 38° C. and p5E8 (VH/VL) to be slightly lower at 34° C. (FIG. 7). Given the remarkably low T₅₀ values of both scFvs and the observation that of the two p5E8 (VL/VH) was slightly more thermally stabile, p5E8 (VL/VH) was selected for further stability engineering.

### Example 4

### Construction of p5E8 scFv Molecules with Improved Thermal Stability

Individual variants and libraries were designed to contain desired amino acid replacements in the conventional p5E8 (VL/VH) scFv using oligonucleotides listed in Table 2. In Table 2, each oligonucleotide name gives reference to desired amino acid substitutions at position(s) in VH or VL according to Kabat numbering system. “Rationale” refers to the design method employed. Said methods are described in detail in U.S. patent application Ser. No. 11/725,970. Mutagenic residues are shown as capital letters. Oligonucleotide pairs for introducing VH/VL disulfides are boxed. Abbreviations are: “COMP”—Computational Analysis, “COVAR”—Covariation Analysis, “CONS”—Consensus Scoring, “INTER-VH/VL” Interface Design, and “SS”—VH/VL disulfide bond.

TABLE 2

Oligonucleotides and rationale for construction of
variant p5E8 (VL/VH) scFvs.

| | | | SEQ ID |
|---|---|---|---|
| Oligo_name | Rationale | Sequence^{†} | NO: |
| VH_E6Q | COMP | gaggtgcagctggtgCagtctgggggcggcttg | 28 |
| VH_L11SDG | COMP | gagtctgggggcggcRRCgcaaagcctgggggg | 29 |
| VH_A12VK_ | COVAR | gtctgggggcggcttgRHGMAGcctggggggtccctg | 30 |
| K13QER | | | |
| VH_N32S | CONS | gttcaggttcaccttcAGCaactactacatggac | 31 |
| VH_D35bHSN | INTER | caataactactacatgMRCtgggtccgccaggctc | 32 |
| VH_Q43KR | COVAR | cgccaggctccagggARGgggctggagtgggtc | 33 |
| VH_S49GA | COVAR | gggctggagtgggtcGSCcgtattagtagtagtg | 34 |
| VH_I51M | COMP | gagtgggtctcacgtatGagtagtagtggtgatc | 35 |
| VL_Q37A | COMP | ggtattatttaaattggtatGCCcagaaaccaggaaa | 36 |
| | | ag | |
| VH_D55GS | CONS | gtattagtagtagtggtRGCcccacatggtacgcag | 37 |
| VH_P56STD | CONS | gtagtagtggtgatRVCacatggtacgcagac | 38 |
| VH_W58YN | INTER | gtggtgatcccacaWACtacgcagactccgtg | 39 |
| VH_E72DN | COVAR | gattcaccatctccagaRACaacgccaagaacacac | 40 |
| VH_A74S | COVAR | catctccagagagaacAGCaagaacacactgtttc | 41 |
| VH_F79YSV | COVAR | gccaagaacacactgKHCcttcaaatgaacagc | 42 |
| VH_Q81E | COMP | gaacacactgtttcttGaaatgaacagcctgagag | 43 |
| VH_A84G | COMP | caaatgaacagcctgagaGGCgaggacacggctgtc | 44 |
| VH_V89AGT | COMP | gagctgaggacacggctRSCtattactgtgcg | 45 |
| VH_S94RK | CONS | gtctattactgtgcgARGttgactacagggtctg | 46 |
| VH_V107T | COVAR | ctcctggggccagggaACCctggtcaccgtctcc | 47 |
| VH_L108AGS | COMP | ctggggccagggagtcKSCgtcaccgtctcctcag | 48 |
| VH_T110VS | COVAR | cagggagtcctggtcKYCgtctcctcagtcgac | 49 |
| VL_L11G | COVAR | cagtctccatcttccGGCtctgcatctgtaggg | 50 |
| VL_V15AGS | COMP | ccctgtctgcatctRSCggggacagagtcacc | 51 |
| VL_V19L | COVAR | ctgtaggggacagaCTGaccatcacttgcagg | 52 |
| VL_I21L | COVAR | ggggacagagtcaccCTGacttgcagggcaag | 53 |
| VL_T22S | COMP | gacagagtcaccatcAGCtgcagggcaagtcag | 54 |
| VL_D28SG | CONS | gcagggcaagtcagRGCattaggtattatttaaattg | 55 |
| VL_R30SL | CONS | gcaagtcaggacattMKCtattatttaaattgg | 56 |
| VL_K39A | COVAR | aattggtatcagcagGCCccaggaaaagctcc | 57 |
| VL_K45ER | INTER | ccaggaaaagctcctSRCctcctgatctatgttg | 58 |
| VL_V50n | INTER | ctaagctcctgatctatNNKgcatccagtttgcaaag | 59 |
| VL_L54R | CONS | ctatgttgcatccagtCGCcaaagtggggtccc | 60 |
| VL_V58S | COVAR | cagtttgcaaagtgggTCCccatcaaggttcagc | 61 |
| VL_E70D | COVAR | cagtggatctgggacaGACttcactctcaccgtc | 62 |
| VL_V75I | COMP | gagttcactctcaccATCagcagcctgcagcc | 63 |
| VL_P80AG | COMP | cagcagcctgcagRGCgaagattttgcgac | 64 |
| VL_F83AGST | COMP | ctgcagcctgaagatRSCgcgacttattactg | 65 |
| VL_T85D | COVAR | cctgaagattttgcgGACtattactgtctacag | 66 |
| VL_L89AQ | INTER | gcgacttattactgtSMRcaggtttatagtacc | 67 |
| VL_R96LY | INTER | gtttatagtacccctMWAacgttcggccaaggg | 68 |
| VL_F98W | INTER | gtacccctcggacgTGGggccaagggaccaag | 69 |
| VL_I106AGS | COMP | gggaccaaggtggaaKSCaaaggcggtggcggg | 70 |
| VH_L45C | SS | gctccagggcaggggTGCgagtgggtctcacg | 71 |
| VL_F98C | SS | gtacccctcggacgTGCggccaagggaccaag | 72 |
| VH_D101C | SS | cttgactacagggtctTGCtcctggggccagggag | 73 |
| VL_L46C | SS | ggaaaagctcctaagTGCctgatctatgttgc | 74 |
| VH_S102C | SS | gactacagggtctgacTGCtggggccagggagtc | 75 |
| VL_L46C | SS | ggaaaagctcctaagTGCctgatctatgttgc | 76 |
| VH_G44C | SS | caggctccagggcagTGCctggagtgggtctcac | 77 |
| VL_Q100C | SS | cctcggacgttcggcTGCgggaccaaggtggaaatc | 78 |

^{†}Positions targeted for mutagenesis are indicated by underline. Ambiguous bases are abbreviated as follows: W = A or T, V = A or C or G, Y = C or T, S = C or G, M = A or C, N = A or C or G or T, R = A or G, K = G or T, B = C or G or T (J Biol Chem. 261(1):13-7 (1986)).

Individual transformed colonies were picked into deep-well 96 well dishes, processed, and screened according to the methods detailed in U.S. patent application Ser. No. 11/725,970. Transformants were grown overnight in expression media consisting of SB (Teknova, Half Moon Bay, Calif. Cat. #S0140) supplemented with 0.6% glycine, 0.6% Triton X100, 0.02% arabinose, and 50 μg/ml carbenicillin at either 30° C. or 32° C.

Each library was screened in duplicate using a thermal challenge assay with supernatant from one replicate subjected to treatment conditions and the second supernatant serving as untreated reference. After thermal challenge, the aggregated material was removed by centrifugation and assayed in the soluble CD23 DELFIA as described in Example 3.

Assay data was processed using Spotfire DecisionSite software (Spotfire, Somerville, Mass.) and expressed as the ratio of the DELFIA counts observed at challenge temperature to the reference temperature for each clone. Clones that reproducibly gave ratios greater than or equal to twice what was observed for the parental plasmid were considered hits. Plasmid DNAs from these positive clones were isolated by mini-prep (Wizard Plus, Promega, Madison, Wis.) and retransformed back into E. coli W3110 for confirmation secondary thermal challenge assays as well as for DNA sequence determination.

Primary and confirmatory results from these assays are shown in Table 3. Several of the stabilized scFv molecules of the invention resulted in improvements in binding activity (T₅₀>38° C.) as compared with the conventional p5E8 scFv. In particular, the T₅₀ values of p5E8 library position V_{H}6 (E6Q), library position V_{H}49 (S49G and S49A), library position V_{H}43 (Q43K), library positions V_{H}72 (E72D and E72N), and library position V_{H}79 (F79S), exhibited increases in thermal stability ranging from +4° C. to +5° C. relative to the conventional p5E8 scFv. One of the stabilizing mutations, VH P56H, serendipitously arose from a PCR error and exhibited an increase in thermal stability of +12° C. relative to the conventional p5E8 scFv. The T₅₀ values of p5E8 library position V_{L}75 (V75I), library position V_{L}80 (P80S), and library positions V_{L}83 (F83A, F83G, F83S and F83T), exhibited increases in thermal stability ranging from +4° C. to +7° C. relative to the conventional p5E8 scFv.

In addition, the T₅₀ values of p5E8 library position V_{H}32 (N32S) and library position V_{H}79 (F79Y) exhibited increases in thermal stability of +2° C. relative to the conventional p5E8 scFv. Combining V_{H}6Q and VH32S mutations with V_{H}49G, V_{H}72D, or V_{H}49A stabilizing mutations enhanced the thermal stability (T₅₀) of p5E8 up to 53° C., an increase of 15° C. relative to the conventional p5E8 scFv.

TABLE 3
p5E8 VH and VL library positions, library
composition, and screening results.

| | | Hit Seq. | ΔT₅₀ |
|---|---|---|---|
| Position | Library | Observed | ° C. |
| VH6 | Q | E6Q | +4 |
| VH32 | S | N32S | +2 |
| VH49 | S, A | S49G, S49A | +5, +5 |
| VH43 | K, R | Q43K | +4 |
| VH72 | D, N | E72D, E72N | +5, +4 |
| VH79 | S, V, Y | F79S, F79Y | +4, +2 |
| VL50 | All amino acids | V50D, V50S | +4, +3 |
| VL75 | I | V75I | +5 |
| VL80 | S, G | P80S | +4 |
| VL83 | S, A, G, T | F83S, F83A, | +4, +6, |
| | | F83G, F83T | +7, +6 |

Table 4 shows the results of a comprehensive thermal stability analysis of the various individual and combined stabilizing mutations introduced into a conventional scFv. Stabilizing mutations were identified that upon combination exhibited increases in thermal stability ranging from +10° C. to +15° C. relative to the conventional p5E8 scFv.

TABLE 4
Characteristics of p5E8 constructs used to produce variant
proteins and T₅₀ results from thermal challenge assay.

| | Linker | | T₅₀ |
|---|---|---|---|
| Plasmid | Length (aa) | Mutation | ° C. |
| pIEH162 | 15 | na | 34 |
| pIEH163 | 15 | na | 38 |
| pIEH164 | 20 | na | 37 |
| pIEH165 | 20 | na | — |
| pIEH171 | 15 | VH E6Q | 42 |
| pIEH172 | 15 | VH N32S | 40 |
| pIEH173 | 15 | VH S49G | 43 |
| pIEH174 | 15 | VH E72D | 43 |
| pIEH175 | 15 | VH Q43K | 42 |
| pIEH176 | 15 | VH S49A | 45 |
| pIEH177 | 15 | VH E72N | 42 |
| pIEH178 | 15 | VH F79S | 42 |
| pIEH179 | 15 | VH F79Y | 40 |
| pIEH187 | 15 | VL V75I | 43 |
| pIEH188 | 15 | VL P80S | 42 |
| pIEH189 | 15 | VL F83S | 42 |
| pIEH190 | 15 | VL F83A | 44 |
| pIEH191 | 15 | VL F83G | 45 |
| pIEH192 | 15 | VL F83T | 44 |
| pIEH-195 | 15 | VL V50D | 43 |
| pIEH-196 | 15 | VL V50S | 41 |
| pIEH-197 | 15 | VH E6Q, S49G | 51 |
| pIEH-198 | 15 | VH E6Q, N32S, S49G | 53 |
| pIEH-199 | 15 | VH E6Q, E72N | 48 |
| pIEH-200 | 15 | VH E6Q, N32S, E72N | 50 |
| pIEH-201 | 15 | VH E6Q, N32S, E72D | 50 |
| pIEH-202 | 15 | VH E6Q, E72D | 48 |
| pIEH-203 | 15 | VH E6Q, N32S, S49A | 51 |
| pIEH-204 | 15 | VH E6Q, S49A | 48 |
| pIEH-236 | 15 | VH P56H | 50 |

na = not applicable
aa = amino acids

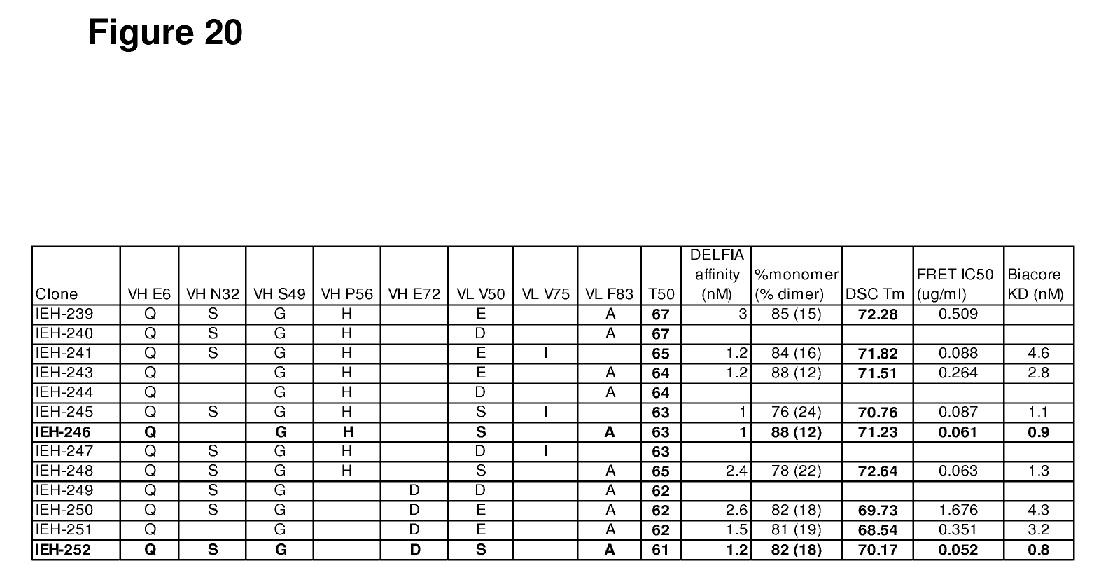

Constructs consisting of various permutations of stabilizing mutations VH E6Q, VH N32S, VH S49G, VH P56H, VH E72D and VL V50E/D/S, VL V75I, and VL F83A were built to identify combinations that further enhanced thermal stability. Constructs were transfected into E. coli strain W3110, mutant p5E8 scFv proteins were produced and purified as described in U.S. patent application Ser. No. 11/725,970. Purified variant p5E8 scFv proteins were tested for: 1) binding affinities to soluble CD23 antigen by DELFIA and FRET immunoassays and Biacore analysis, 2) Percentage monomers/dimers by size exclusion HPLC (Agilent Technologies) with static light scattering and refractive index detectors (MiniDAWN/ReX, Wyatt Technology), and 3) Tm's (midpoint of thermal unfolding) by differential scanning calorimetry (capDSC, MicroCal, LLC). FIG. 20 summaries the results of these biochemical and biophysical analyses and shows that combining select stabilizing mutations enhances the thermodynamic stability of p5E8 scFv with no loss in binding affinity. Combining stabilizing mutations exhibited increases in thermal stability ranging from +23° C. to +29° C. relative to the conventional p5E8 scFv (Table 4, pIEH163). Based on a combination of desirable sequence, stability, and affinity properties, constructs IEH-246 and IEH-252 were selected for construction of stable p5E8 tetravalent antibodies.

### Example 5

### Production of Stabilized p5E8 Tetravalent Antibodies

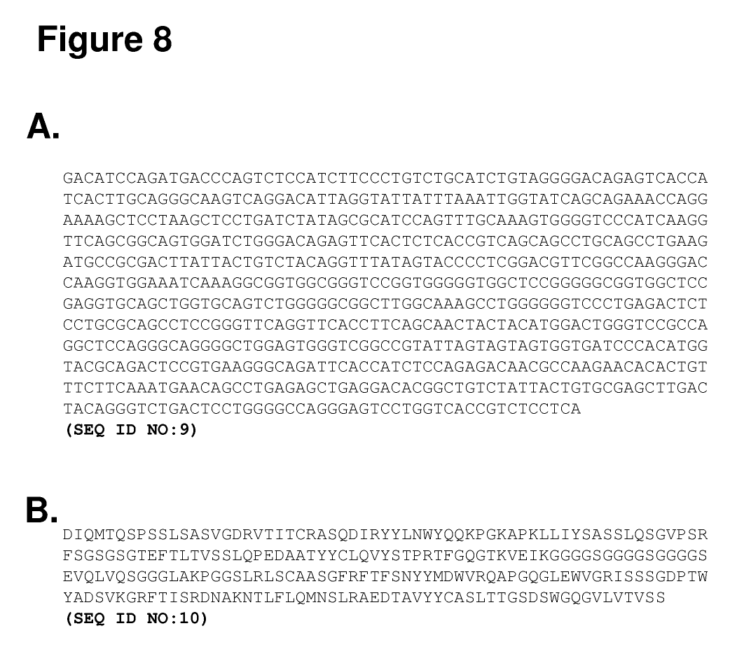

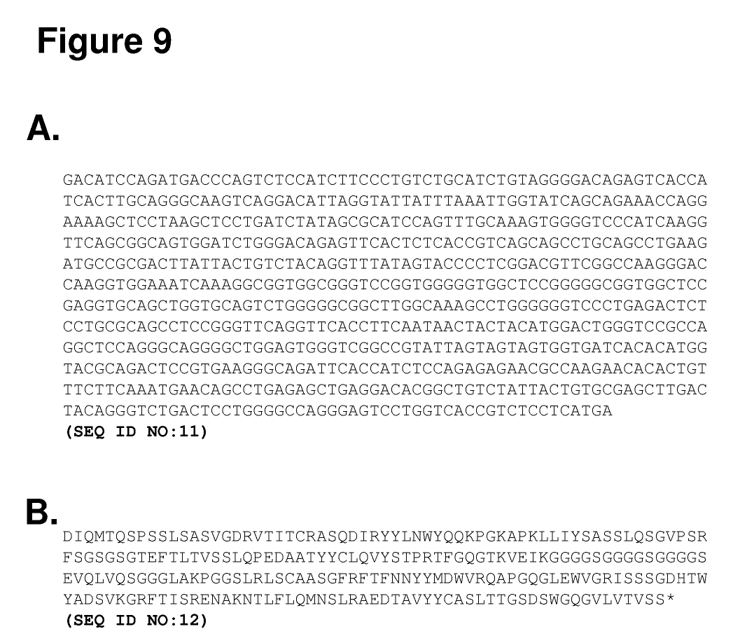

Stabilized p5E8 IEH-252 and IEH-246 scFvs of the invention are used to construct tetravalent antibodies as both N-terminal and C-terminal scFv fusions as shown in FIG. 2A. DNA and amino acid sequences of p5E8 IEH-252 scFv are shown in FIGS. 8A and 8B, respectively. DNA and amino acid sequences of p5E8 IEH-246 scFv are shown in FIGS. 9A and 9B, respectively.

A. Construction of N-terminal p5E8 Tetravalent Antibodies

The IEH-252 and IEH-246 p5E8 scFv DNAs described in Example 4 were used to construct N-terminal p5E8 tetravalent antibodies using methods similar to that described in U.S. patent application Ser. No. 11/725,970. PCR amplification was performed using the oligonucleotide primers described in Table 5. A (Gly₄Ser)₅ linker was used to connect the stabilized p5E8 scFvs to the mature amino terminus of PRIMATIZED® p5E8 IgG heavy chain. The p5E8 IgG vector was first modified by PCR to remove an unwanted BamH I site 5′ of the carboxyl end of p5E8 IgG and then further modified to introduce a BamH I site and a portion of the (Gly₄Ser)₅ linker to facilitate cloning of stabilized p5E8 scFvs at the amino terminus. This was accomplished by first PCR amplifying heavy chain gene sequences from the Mlu I site to the carboxyl end BamH I site as a single PCR fragment with the forward 5′-PCR primer 078-F3 (includes a Mlu I restriction endonuclease site followed by a BamH I site and sequence encoding a portion of the (Gly₄Ser)₅ linker and the amino terminus of p5E8 VH) and the reverse 3′ PCR primer 078-R3 (includes the carboxyl terminus of G1 and a Bgl II site). The PCR fragment was digested with Mlu I and Bgl II restriction endonucleases and was ligated to the Mlu I/BamH I digested p5E8 IgG antibody vector. Correct orientation was confirmed by restriction analysis and DNA sequence analysis confirmed the correct sequence of the construct.

DNA sequences from stabilized IEH-252 and IEH-246 p5E8 scFvs were amplified by PCR using the forward 5′ p5E8 scFv VL PCR primer 078-F1 (includes a Mlu I restriction endonuclease site followed by sequence encoding the last three amino acids of the heavy chain signal peptide followed by sequences encoding the amino terminus of p5E8 scFv VL) and the reverse 3′ p5E8 scFv VH PCR primer 078-R4 (includes the carboxyl terminus of p5E8 scFv VH followed by a portion of the (Gly₄Ser)₅ linker and a BamH I site). The PCR fragment was digested with Mlu I and BamH I restriction endonucleases and ligated to the Mlu I/BamH I digested p5E8 IgG antibody vector.

This resulted in a fusion product of the stabilized p5E8 scFvs to the amino terminus of the p5E8 antibody VH domain through a 25 amino acid (Gly₄Ser)₅ linker. The ligation mixtures are used to transform E. coli strain TOP 10 competent cells (Invitrogen Corporation, Carlsbad, Calif.). E. coli colonies transformed to ampicillin drug resistance are screened for presence of inserts. DNA sequence analysis is used to confirm the correct sequence of the final constructs pXWU104 encoding N-terminal p5E8-252 tetravalent antibody and pXWU078 encoding N-terminal p5E8-246 tetravalent antibody.

TABLE 5

Oligonucleotides for construction of N-terminal
p5E8 tetravalent antibodies with stabilized
p5E8 scFvs. Restriction endonuclease sites are
shown underlined.

| | |
|---|---|
| 078F3 | 5′-GTTGCTACGCGTGGCGGTGGATCCGGGGGAGGT |
| (SEQ ID NO:79) | GGCTCCGAGGTGCAGCTGGTGGAGTCTGG-3′ |
| 078-R3 | 5′-GTTAACAGATCTTCATTTACCCGGAGACAGGGA |
| (SEQ ID NO:80) | GAGG-3′ |
| 078-F1 | 5′-GTTGCTACGCGTGTCCTGTCCGACATCCAGATG |
| (SEQ ID NO:81) | ACCCAGTCTC-3′ |
| 078-R4 | 5′-TCCCCCGGATCCACCGCCCCCTGAACCGCCCCC |
| (SEQ ID NO:82) | TCCAGAGCCCCCTCCACCGGACCCTCCACCGCCT |
| | GAGGAGACGGTGACCAG-3′ |

B. Construction of C-terminal p5E8 Tetravalent Antibody

The IEH-246 and IEH-252 p5E8 scFvs DNAs described in Example 4 were used to construct C-terminal p5E8 tetravalent antibodies using methods similar to that described in U.S. patent application Ser. No. 11/725,970. PCR amplification was performed using the oligonucleotide primers described in Table 6. A Ser(Gly₄Ser)₃ linker was used to connect the stabilized p5E8 scFvs to the carboxyl terminus of PRIMATIZED® p5E8 IgG heavy chain. DNA sequences from stabilized IEH-252 and IEH-246 p5E8 scFvs were amplified by PCR using the forward 5′ VL PCR primer C-23VL-F (includes a BamH I restriction endonuclease site followed by sequence encoding a portion of the Ser(Gly₄Ser)₃ linker peptide and the amino terminus of p5E8 scFv VL) and the reverse 3′ VH PCR primer C-23VH-R (includes the carboxyl terminus of p5E8 scFv VH and a stop codon followed by a Bgl II site). The PCR products were gel isolated, digested with BamH I and Bgl II restriction endonucleases and ligated to a BamH I digested p5E8 IgG antibody vector. This resulted in a fusion product of the stabilized p5E8 scFvs to the carboxyl terminus of the p5E8 antibody CH3 domain through a 16 amino acid Ser(Gly₄Ser)₃ linker. The ligation mixtures are used to transform E. coli strain TOP 10 competent cells (Invitrogen Corporation, Carlsbad, Calif.). E. coli colonies transformed to ampicillin drug resistance are screened for presence of inserts. DNA sequence analysis is used to confirm the correct sequence of the final constructs pXWU103 encoding C-terminal p5E8-252 tetravalent antibody and pXWU077 encoding C-terminal p5E8-246 tetravalent antibody.

TABLE 6

Oligonucleotides for construction of C- terminal
p5E8 tetravalent antibodies with stabilized p5E8
scFvs. Restriction endonuclease sites are shown
underlined.

| | |
|---|---|
| C-23VL-F | 5′-AGAGAGGGATCCGGTGGAGGGGGCTCCGGCGGT |
| (SEQ ID NO:83) | GGCGGGTCCGACATCCAGATGACCCAGTC-3′ |
| C-23VH-R | 5′-AGAGAGAGATCTTCATGAGGAGACGGTGACCAG |
| (SEQ ID NO:84) | GAC-3′ |

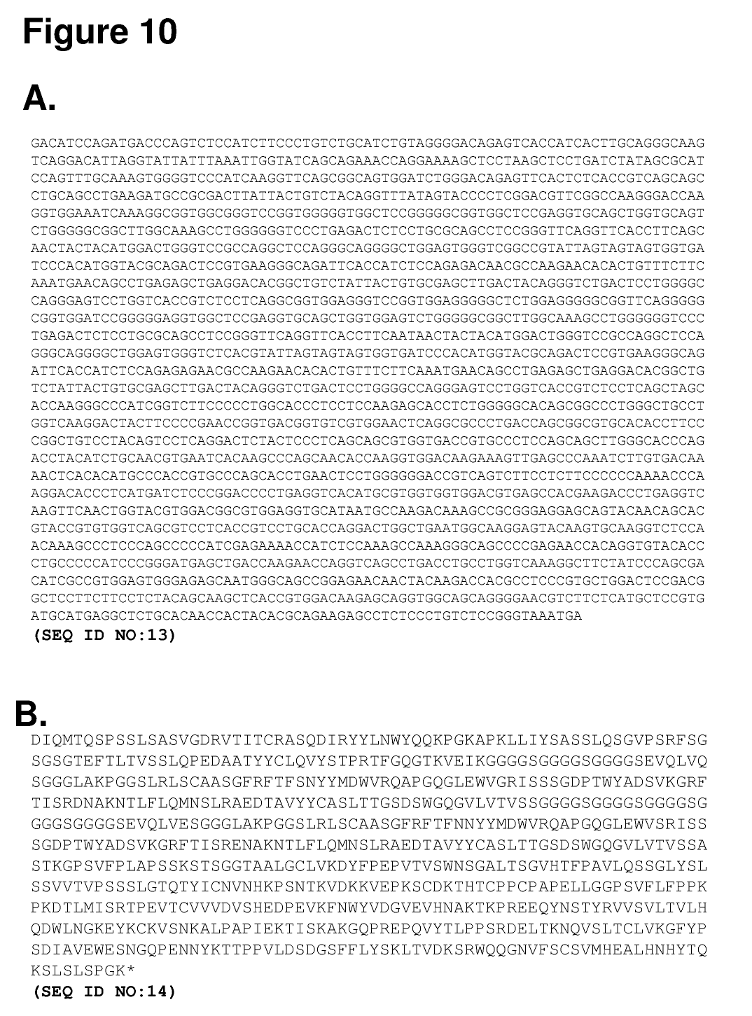

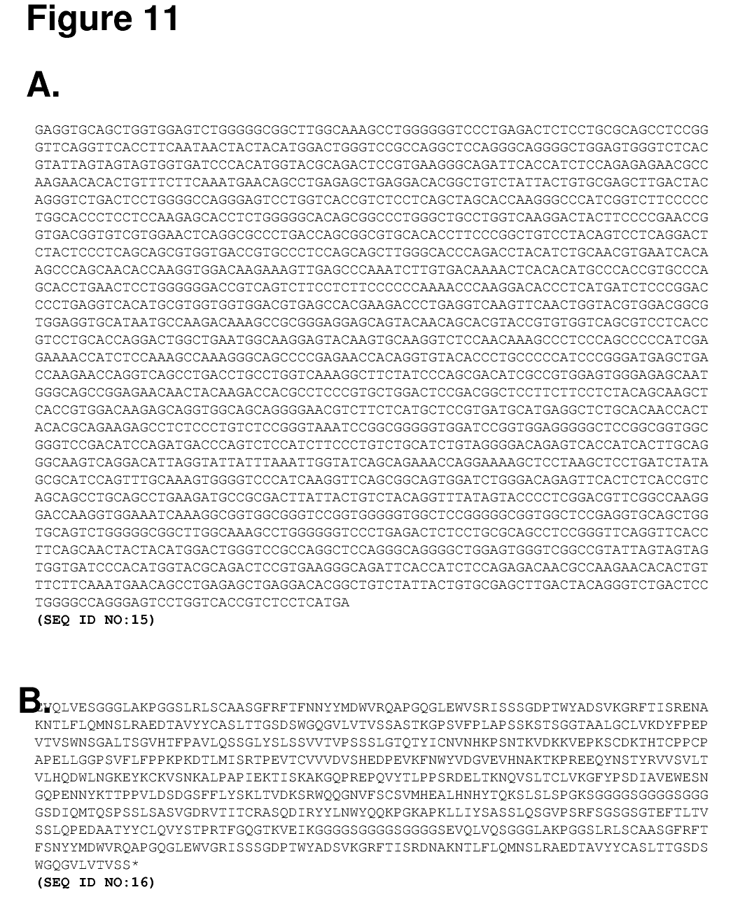

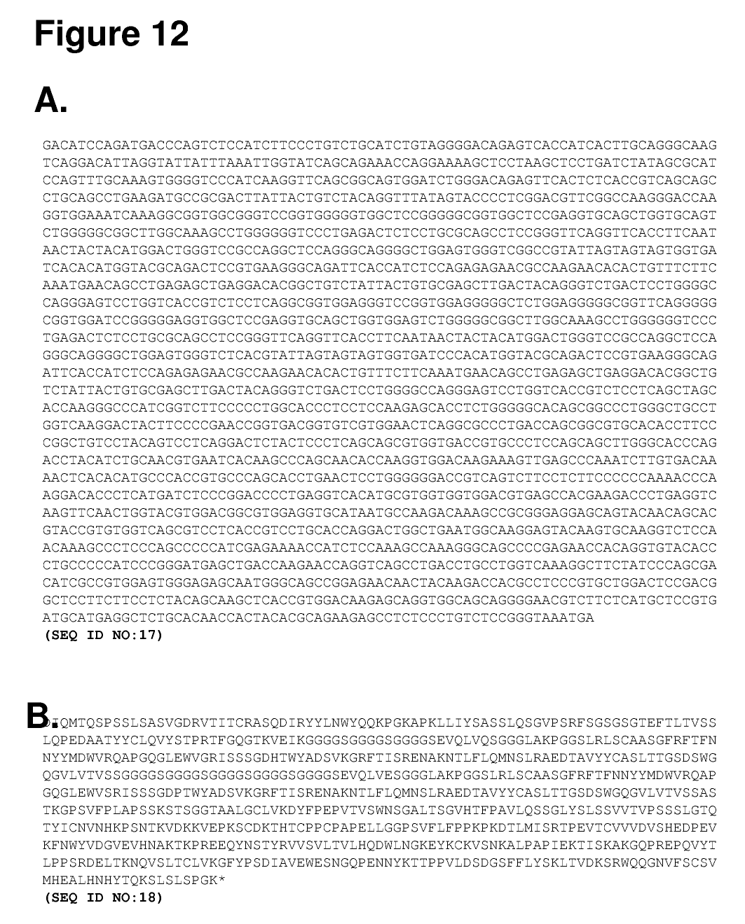

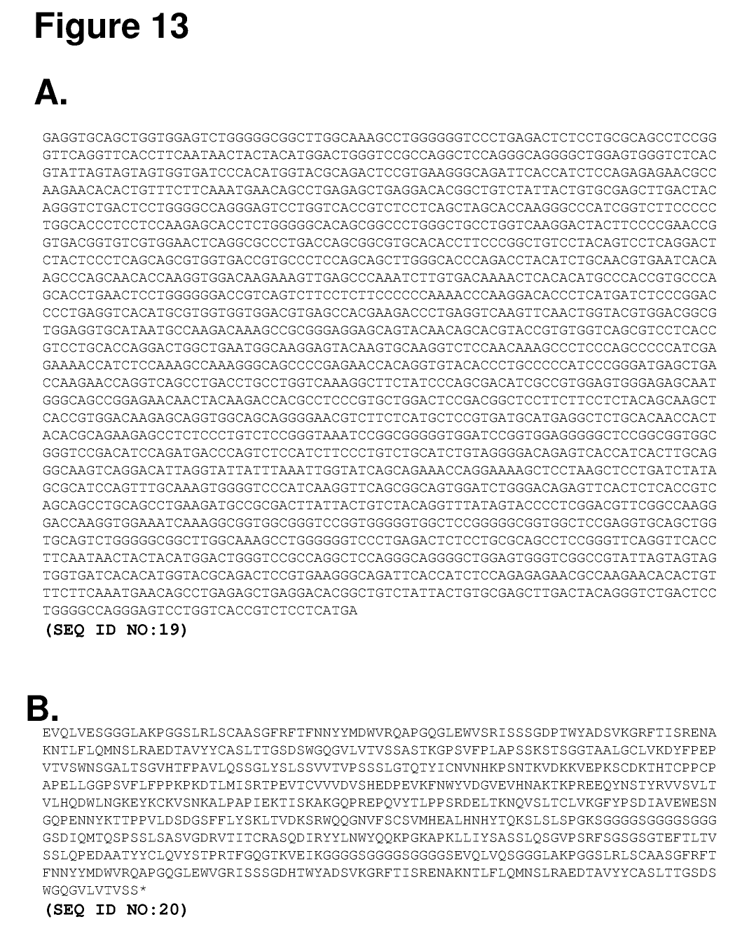

The PRIMATIZED® p5E8 light chain used is common among all the N- and C-tetravalent antibodies and DNA and amino acid sequences are shown in FIGS. 4A and 4B, respectively. Heavy chain DNA and amino acid sequences for N-terminal tetravalent p5E8 comprising the stabilized pIEH252 scFv are shown in FIGS. 10A and 10B, respectively. Heavy chain DNA and amino acid sequences for C-terminal tetravalent p5E8 comprising the stabilized pIEH252 scFv are shown in FIGS. 11A and 11B, respectively. Heavy chain DNA and amino acid sequences for N-terminal tetravalent p5E8 comprising the stabilized pIEH246 scFv are shown in FIGS. 12A and 12B, respectively. Heavy chain DNA and amino acid sequences for C-terminal tetravalent p5E8 comprising the stabilized pIEH246 scFv are shown in FIGS. 13A and 13B, respectively.

C. Stable Expression of Stabilized p5E8 Tetravalent Antibodies in CHO cells, Antibody Purification, and Characterization

Plasmid DNAs pXWU077, pXWU078, pXWU103, and pXWU104 were used to transform DHFR-deficient CHO DG44 cells for stable production of antibody protein. Transfected cells were grown in alpha minus MEM medium containing 2 mM glutamine supplemented with 10% dialyzed fetal bovine serum (Invitrogen Corporation) and enriched as a stable bulk culture pool using fluorescently labeled antibodies and reiterative fluorescent-activated cell sorting (FACS) (Brezinsky, et al. J Immunol Methods. 277(1-2):141-55 (2003)). FACS was also used to generate individual cell lines. Cell pools or cell lines were adapted to serum-free conditions and scaled for antibody production.

i. C-Tetravalent p5E8 Antibody

25 L of C-Tetravalent p5E8 (pXWU103) supernatant from a 10 day bioreactor run was harvested and precleared by ultrafiltration. The tetravalent antibody was captured from the supernatant using Protein A Sepharose FF (GE Healthcare). The tetravalent molecule was eluted from the Protein A using 0.1 M glycine at pH 3.0, neutralized with Tris base, dialyzed into PBS, and further purified using preparative size exclusion chromatography (Superdex 200, GE Healthcare). C-Tetravalent p5E8 was dialyzed into PBS. Endotoxin levels were assayed by kinetic quantitative chromogenic LAL Analysis using the EndoSafe® PTC kit (Charles River Labs). Purity and percentage of monomer tetravalent antibody product was assessed by 4-20% Tris-glycine SDS-PAGE and analytical size-exclusion HPLC, respectively. The process yielded 871.4 mg C-Tetravalent p5E8 protein at a concentration of 11.2 mg/ml, >99% purity, with a residual endotoxin concentration of 0.105 EU/mg protein.

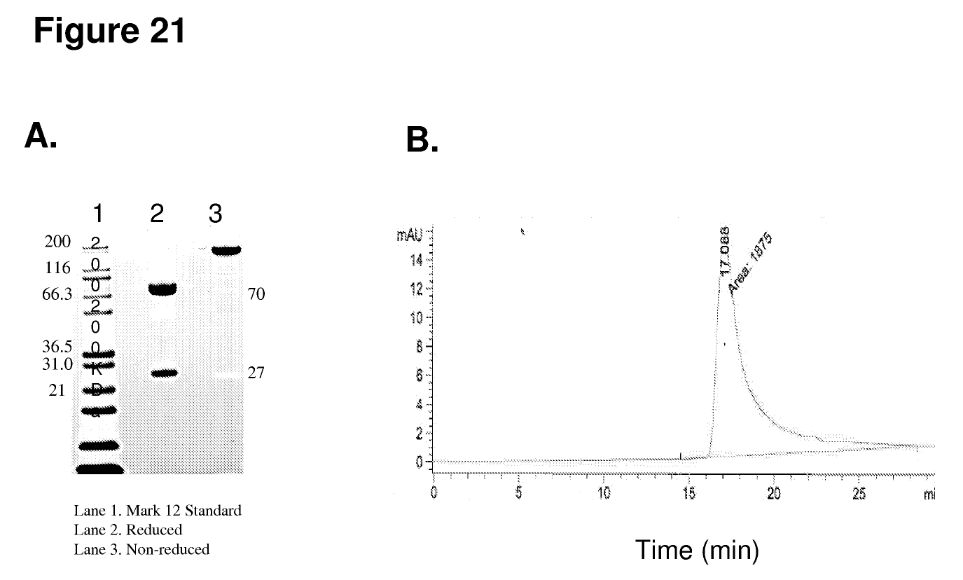

FIG. 21A shows an SDS-PAGE gel of purified stability-engineered C-Tetravalent p5E8 (pXWU103). The reduced lane shows the expected sizes of the heavy and light chain proteins. Importantly, there is no significant level of degraded or unwanted lower molecular weight byproducts detected. FIG. 21B shows an analytical SEC elution profile of purified stability-engineered C-Tetravalent p5E8 (pXWU103). This analysis demonstrates that the stability-engineered C-Tetravalent p5E8 is essentially >99% pure, monomeric, and free of higher order molecular weight species.

ii. N-Tetravalent p5E8 Antibody

600 mL of N-Tetravalent p5E8 (pXWU104) supernatant from a ˜10 day shake-flask run was harvested and precleared by ultrafiltration. The N-Tetravalent p5E8 was purified as described above for the C-Tetravalent p5E8. Purity and percentage of monomer tetravalent antibody product was assessed by 4-20% Tris-glycine SDS-PAGE and analytical size-exclusion HPLC, respectively. The process yielded 11 mg N-Tetravalent p5E8 protein at a concentration of 2.5 mg/ml, >99% purity. Endotoxin level was not determined.

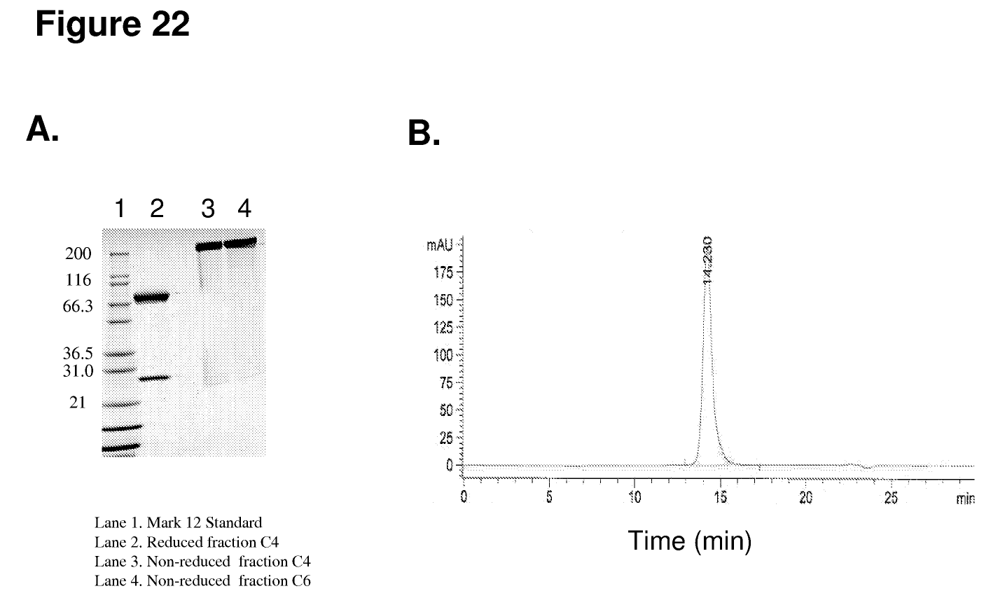

FIG. 22A shows an SDS-PAGE gel of purified stability-engineered N-Tetravalent p5E8 (pXWU104). The reduced lane shows the expected sizes of the heavy and light chain proteins. Again, there is no significant level of degraded or unwanted lower molecular weight byproducts detected. FIG. 22B shows an analytical SEC elution profile of purified stability-engineered N-Tetravalent p5E8 (pXWU104). This analysis demonstrates that the stability-engineered N-Tetravalent p5E8 is essentially >99% pure, monomeric, and free of higher order molecular weight species.

### Example 6

### Stabilized p5E8 Tetravalent Antibodies Bind to Four CD23 Molecules

N-terminal (pXWU104) and C-terminal (pXWU103) p5E8 tetravalent antibodies have four antigen binding sites. Both the C-terminal and N-terminal tetravalent antibodies were tested with two separate methods to determine whether the antibodies are able to bind four CD23 molecules simultaneously.

### A. Solution Phase Biacore Experiments

First, the binding was analyzed using solution phase surface plasmon resonance (Day E S, Cachero T G, Qian F, Sun Y, Wen D, Pelletier M, Hsu Y M, Whitty A. Selectivity of BAFF/BLyS and APRIL for binding to the TNF family receptors BAFFR/BR3 and BCMA. Biochemistry. 2005 Feb. 15; 44(6):1919-31.) The method utilizes conditions referred to as “mass-transport-limited” binding, in which the initial rate of ligand binding (protein binding to the sensor chip) is proportional to the concentration of ligand in solution (BIApplications Handbook (1994) Chapter 6: Concentration measurement, pp 6-1-6-10, Pharmacia Biosensor AB). Under these conditions, binding of the soluble analyte (protein flowing over chip surface) to the immobilized ligand is fast compared to the diffusion of the analyte into the dextran matrix on the chip surface. Therefore, the diffusion properties of the analyte and the concentration of analyte in solution flowing over the chip surface determine the rate at which analyte binds to the chip. This initial rate of binding (Vᵢ) is determined by the linear fit to the initial binding phase of the sensorgrams observed over the first few seconds of association. This initial rate is proportional to the concentration of ligand, and therefore can be used to determine the concentration of free ligand in solution.

In this experiment, the concentration of free CD23 (soluble and monomeric form) in solution is determined by the initial rate of binding to a CM5 Biacore chip containing immobilized p5E8 MAb. Into these CD23 solutions were titrated the p5E8 Fab prepared by papain digestion (one potential binding site), p5E8 MAb (two potential binding sites), and the C-terminal anti-CD23 tetravalent antibody (four potential binding sites). The p5E8 Fab, MAb, and tetravalent antibody are the ligands and CD23 is the analyte being tested. The affinity and stoichiometry of these molecules to CD23 is demonstrated by their ability to inhibit CD23 from binding the immobilized anti-CD23 MAb on the surface of the sensorchip.

The data for the CD23/anti-CD23 antibody mixtures were fitted to a quadratic binding equation:

Vi=m[[L]ₜ−1/2[R−\sqrt{R²−4N[Ab]ₜ[L]ₜ}

[symbol]

R=N[Ab]ₜ+[L]ₜ+K_{D}

Where, m is the slope of the standard curve for CD23 binding to p5E8 monoclonal antibody immobilized on chip surface, [L]ₜ is the concentration of CD23 flowed over chip surface, [Ab]ₜ is the concentration of antibody at that given data point, and N is the number of bound CD23 molecules to antibody. From this equation, the stoichiometry, N, of antigen binding to antibody can be determined. The method measures the distribution of bound versus free CD23 present in each of the preincubated solutions, and therefore the affinity and stoichiometries obtained from the data represent true solution values.

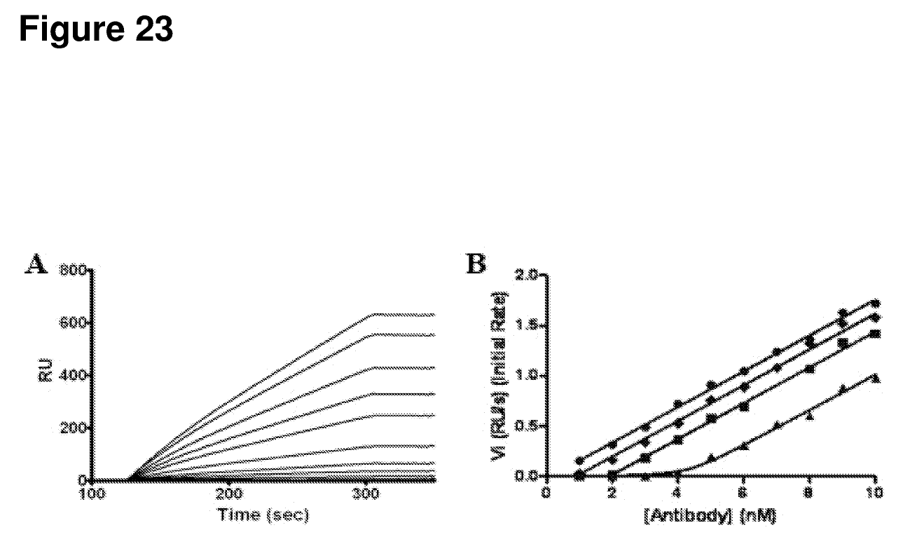

P5E8 was immobilized to 20247 RUs on a CM5 sensor chip. CD23 and CD23 plus antibody were flowed over the sensor chip at a 10 μl/min for 3 minutes. Initial binding rates were obtained from a fit to the initial linear portion of the raw sensorgram data (FIG. 23A). The N (stoichiometry) values were determined by plotting the antibody concentration against the initial rate, Vᵢ, obtained at each concentration (FIG. 23B). Decreases in the initial binding rates are observed for low concentrations of CD23 incubated with antibodies to equilibrium as most of the CD23 in solution is bound to antibody and therefore not available to bind to the p5E8 on the chip surface. At high concentrations of CD23, the amount of CD23 present in solution equals and then exceeds the amount of available antibody. Three experiments measured the initial rates of binding of free CD23 to the p5E8 sensor chip (concentrations for CD23 ranged from 0.15 to 15 nM) in the presence of constant concentrations of antibody of 1, 2.5 and 10 nM. The results are shown in Table 7 and reported as the average of the three titrations. The calculated stoichiometries correspond to binding of CD23 antigen to all four available antigen binding sites for the C-terminal tetravalent p5E8. The bivalent p5E8 antibody bound two molecules of CD23 and the monovalent Fab fragment prepared from p5E8 IgG was found to bind a single molecule of CD23.

TABLE 7
Solution Phase Biacore experiments

| Molecule | N +/− Std Dev |
|---|---|
| C-terminal tetravalent | 4.16 +/− 0.20 |
| p5E8 | |
| p5E8 IgG | 2.11 +/− 0.08 |
| p5E8 Fab | 1.05 +/1 0.04 |

### B. Isothermal Titration Calorimetry Experiments

Isothermal titration calorimetry (ITC) accurately measures the thermodynamics of protein-protein interactions and can further provide the stoichiometry of the experimental complex. CD23, C-terminal tetravalent p5E8, p5E8 IgG and p5E8 Fab were extensively dialyzed for 12 hours against PBS. ITC experiments were performed on an iTC200 microcalorimeter (MicroCal LLC, Northampton, Mass.). After degassing, 25×1.5 μl aliquots of 100 μM CD23 were injected at 5 minute intervals into the 350-μl sample cell containing 5 μM solutions of antibody/Fab. Raw data were then integrated and fitted using a nonlinear least squares routine in ITC data analysis software ORIGIN (MicroCal LLC, Northampton, Mass.). The fitting generated values for the independent variables ΔH⁰ (binding enthalpy), N (the stoichiometry), and K_{b} (binding constant). For each antibody/Fab, ITC experiments were performed at 5° C., 15° C., 25° C., and 37° C. in order to determine the temperature dependence on the binding heats and to optimize the experiments to obtain better signal to noise.

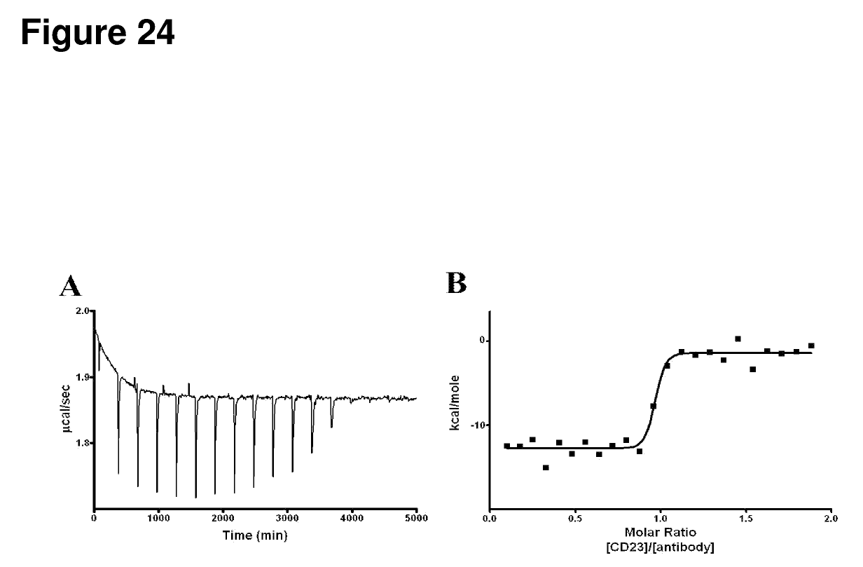

Representative ITC data for the p5E8 Fab and CD23 are shown in FIG. 24. The CD23 in the syringe and the antibody in the sample cell are both monomers in solution. The stoichiometry (N) values for the ITC experiments are summarized in Table 8. On fitting the data, N values approximating 1, 2, and 4 would indicate binding of all available antigen binding sites for the p5E8 Fab, p5E8 IgG and the C-terminal tetravalent p5E8, respectively. The data suggests that the tetravalent p5E8 binds four CD23 antigens simultaneously or one CD23 antigen to each of the four antigen binding sites. The N=3 for p5E8 was higher than expected for a typical antibody (N≈2, for divalent antibody). Small fluctuations around the expected stoichiometry might be expected based on limitations for the determination of the concentrations of both the ligand and the antibody. The N=1 for the p5E8 Fab corresponds with a single antigen binding site on the Fab.

TABLE 8
Stoichiometry (N) values for the binding of CD23 to
C-terminal tetravalent p5E8, P5E8 IgG, and P5E8 Fab
as measured by isothermal titration calorimetry.

| | Temperature | | | | |
|---|---|---|---|---|---|
| N | 5° C. | 15° C. | 25° C. | 37° C. | Avg. N |
| C-tetravalent p5E8 | 4.24 | 4.41 | 4.28 | 4.68 | 4.41 |
| p5E8 IgG | — | 2.70 | 3.05 | 3.46 | 3.07 |
| p5E8 Fab | 1.16 | 0.83 | 0.95 | 1.04 | 0.99 |

Results from these two experiments demonstrate that the tetravalent antibody format as embodied in the C-terminal (pXWU103) p5E8 tetravalent antibody is capable of binding antigen at all four antigen binding sites.

### Example 7

### Stabilized p5E8 Tetravalent Antibodies binds to FcγRIII

An AlphaScreen (Amplified Luminescent Proximity Homogeneous Assay) was utilized to determine binding of N-terminal (pXWU104) and C-terminal (pXWU103) p5E8 tetravalent antibodies to a GST-tagged Fc receptor FcγRIIIa (V158 allotype). p5E8 and human IgG were used as controls. The wild-type p5E8 antibody (conjugated to acceptor beads) and FcγRIII (V158) (bound to donor beads) interact and produce a signal at 520-620 nm. Addition of un-conjugated antibody competes with the wild-type IgG/FcγRIII interaction, reducing the fluorescence quantitatively to allow determination of relative binding affinities.

Starting with 100 μL of the tested antibodies at 1.2 mg/mL concentration (final concentration for antibody is 0.24 mg/ml), two-fold serial dilutions (total 12 dilutions) of the tested antibodies were plated in a 96 well V-bottom plate (Costar, Cat#3897) at 5 times the final concentration. Each dilution was done in duplicate. 5 μl of each dilution was added to a different assay 96 well V-bottom plate. 10 of stock biotin-mouse anti-GST (Clone 8-326, Calbiochem, Cat#OB03SP1) and GST-tagged Fc receptor FcγRIIIa (V158) were added per well for final concentrations of 1.25 μg/ml. The plate was then sealed and incubated at room temperature for 40 minutes. A master mix consisting of a control human IgG1 conjugated to acceptor beads at 20 μg/ml final concentration and streptavidin labeled donor beads at 20 μg/ml final concentration was made. 10 of this acceptor/donor bead mix was added to each well. The plate was sealed and incubated at room temperature for 1 hour. The plate was read on a FusionAlpha plate reader (PerkinElmer, Waltham, Mass.) and the data analyzed using Prism version 5 software (GraphPad Software, La Jolla, Calif.) using a log (inhibitor) vs. response with a constant slope as the model. The values obtained for the mid-point of the inhibitor curves are referred to as IC₅₀ values.

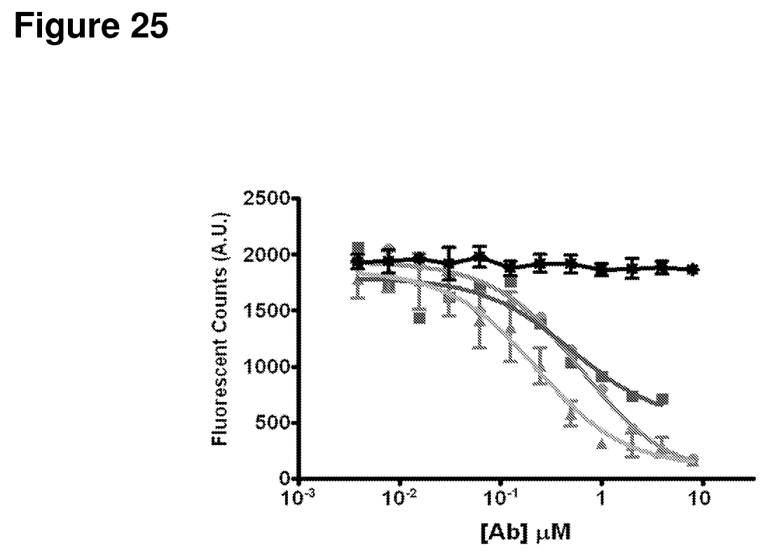

The tetravalent p5E8 constructs were screened for their relative FcγRIII (V158) affinity by using an AlphaScreen assay. The fits to the binding data provide the inhibitory concentration 50% (IC₅₀) for each antibody allowing a relative comparison between the tetravalent constructs and p5E8 (FIG. 25). The IC₅₀ values are comparable between p5E8 (0.63 μM) and the N-terminal tetravalent p5E8 (0.49 μM) and C-terminal tetravalent p5E8 (0.23 μM). An aglycosylated human IgG4 antibody, which does not bind FcγRIII (V158), was used as a negative control. The data show that the tetravalent architecture does not interfere with the ability for either the N-terminal or C-terminal tetravalent p5E8 Fc to bind to FcγRIII.

### Example 8

### Long-Term Stability of C-Tetravalent p5E8 Antibody

It is highly desirable for a protein therapeutic to have a long shelf life, with minimal changes to the physical or chemical properties of the protein during storage. Therefore, the stability of the C-terminal tetravalent p5E8 antibody (pXWU103) was analyzed over the course of 2.5 months at 4-40° C., and protein aggregation or precipitation was monitored using analytical size exclusion chromatography (SEC). C-terminal tetravalent p5E8, p5E8 IgG and BHA10 (a human IgG1 control) were concentrated to 5 mg/ml after dialysis into the following buffer: 10 mM L-Histidine, pH 6.0, 0.05% w/v polysorbate 80. Protein samples were then aliquoted and placed in storage at 4° C., 25° C. and 40° C. At given time point, samples were run on analytical size exclusion HPLC and the percentage of protein eluted at the expected retention time (17 minutes) for a monomeric species was recorded (Table 9). The control IgG1 antibody is used to determine the retention time for the monomer protein. Aggregated protein eluted at shorter retention times and protein degradation products eluted at longer retention times in the SEC elution profile. Therefore the percentage of monomer species was used to monitor the overall stability of the protein at a given time point.

TABLE 9
Percent monomer detected using analytical
size exclusion chromatography.

| | | Days | | | | |
|---|---|---|---|---|---|---|
| Protein | Temp (° C.) | 1 | 2 | 4 | 42 | 80 |
| C-tetravalent | 4 | 95.2 | 97.0 | 97.3 | 89.8 | 90.5 |
| p5E8 | | | | | | |
| | 25 | 95.5 | 96.6 | 97.7 | 94.1 | 82.2 |
| | 40 | 95.6 | 97.2 | 98.7 | 88.7 | 88.6 |
| p5E8 IgG | 4 | 100 | 100 | 100 | 96.0 | 97.6 |
| | 25 | 100 | 100 | 100 | 96.4 | 95.3 |
| | 40 | 100 | 100 | 100 | 90.8 | 88.6 |
| IgG1 control | 4 | 96.4 | 100 | 100 | 93.6 | 93.5 |
| | 25 | 97.5 | 100 | 100 | 100 | 95.4 |
| | 40 | 98.9 | 100 | 100 | 95.7 | 82.4 |

The C-terminal tetravalent p5E8 behaved as well as p5E8 and the control IgG gaining ˜9% aggregates on average for the three temperatures over the two and a half month period in contrast to ˜6% and ˜7% for p5E8 IgG and the IgG1 control respectively.

### Example 9

### Characterization of Antibody Specificity by Flow Cytometry

Binding affinities of purified antibodies were determined by flow cytometry analysis of CHO cells expressing human CD23 and on CD23+ lymphoma cell lines. In all experiments cells were incubated with test antibodies at a range of concentrations for 45 minutes, washed and then incubated with fluorochrome conjugated secondary antibody. Cells were subsequently washed and stained with Propidium Iodide at 2 μg/ml (Invitrogen, P3566) prior to analysis. Live cells were analyzed using a BD FACSArray and the data analyzed using GraphPad Prism software.

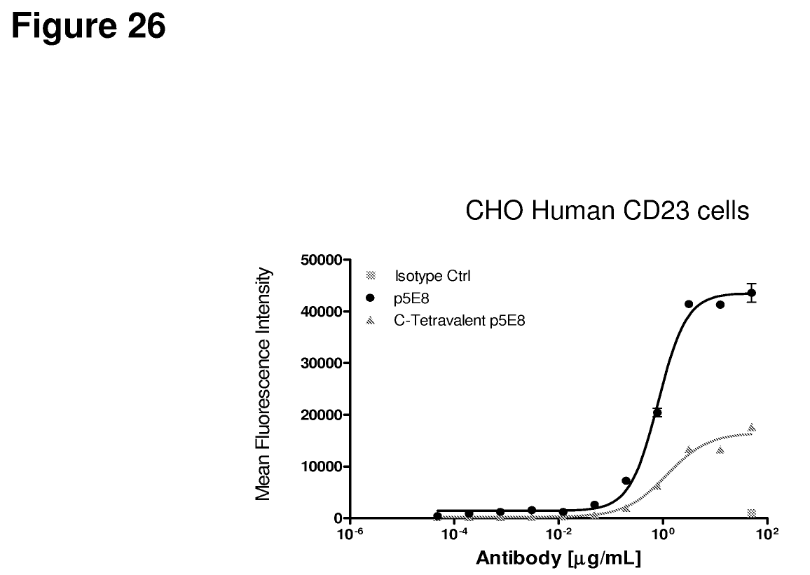

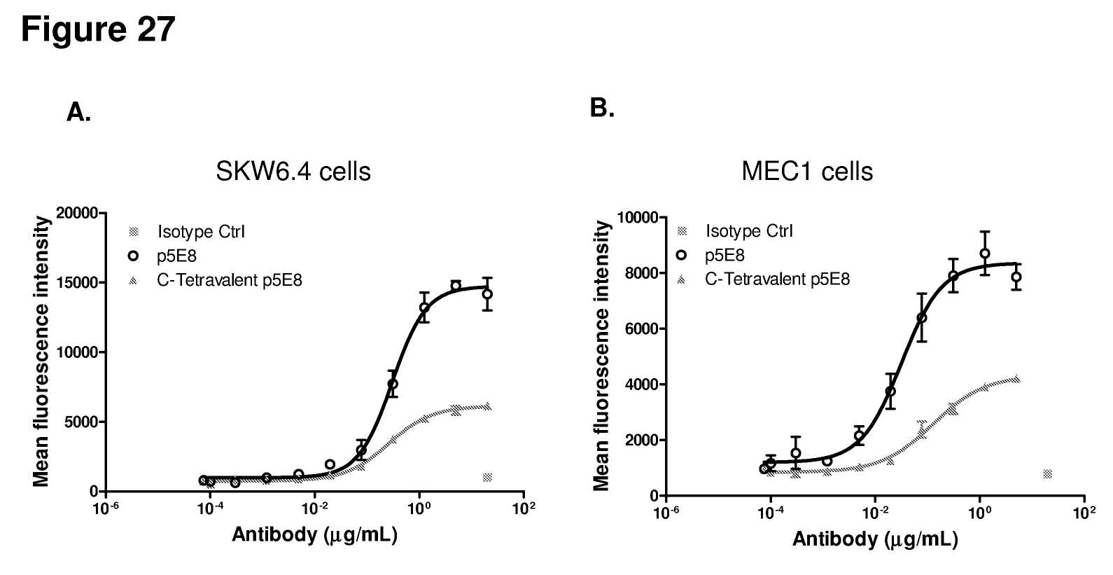

FIG. 26 demonstrates titrations of the p5E8 and tetravalent p5E8 antibodies in CHO-human CD23 cells. No binding was observed on parental CHO cells. Similar binding results were observed in CD23+ human cell lines (FIG. 27) demonstrating that the tetravalent p5E8 antibody displayed similar binding characteristics and binding affinity as the bivalent p5E8 antibody (summarized in Table 10).

TABLE 10
Binding affinity of p5E8 antibodies on CD23+ cell lines

| EC50 values (nM) | p5E8 | C-Tetravalent p5E8 |
|---|---|---|
| CHO Human CD23 cells | 5.4 | 5.91 |
| SKW CD23⁺ B lymphoma cells | 2.0 | 1.3 |
| MEC1 CLL cells | 0.21 | 0.64 |

### Example 10

### Tetravalent p5E8 Antibodies Retain Similar Minimal ADCC Activities as the Parental 5E8 Antibody

Previous studies have demonstrated that the parental p5E8 has weak ADCC activity compared to other IgG1 antibodies such as rituximab. Though the biochemical binding experiment described in Example 7 demonstrated that the N- and C-tetravalent p5E8 antibodies bound to FcγRIII it was not known whether the addition of a CD23 scFv to the C-terminus of p5E8 might alter effector function by affecting the availability of the Fc region of the antibody to Fc gamma receptors (FcγR) expressed by immune cells. To address this question ADCC assays were performed to determine whether the tetravalent 5E8 could be bound by FcγR bearing cells in an in vitro assay.

Human effector cells were prepared from whole blood from one healthy donor and ADCC activity measured in SKW6.4 cells and in four individual CLL donors. Briefly, human peripheral blood mononuclear cells (PBMCs) were purified from heparinized whole blood by standard Ficoll-paque separation (Sigma, Histopaque 1077-1). The cells were resuspended in ATCC RPMI1640 media containing 10% FBS and 200 U/ml of human IL-2 and incubated overnight at 37° C. The following day, the cells were collected and washed once in RPMI1640 media containing 10% FBS and 200 U/ml IL-2 and resuspended at 1×10⁷ cells/ml.

Target cells were incubated with 100 μCi 51Cr for 1 hour at 37° C. The target cells were washed once to remove the unincorporated 51Cr, and plated at a volume of 1×10⁴ cells/well. Target cells were incubated with 50 μl of effector cells and 50 μl of antibody. A target to effector ratio of 1:50 was used throughout the experiments. Four controls were used. These included (a) target cells in medium; (b) target cells in the presence of 1% TRITON-X®-100; (c) CE9.1, a macaque/human chimeric IgG1 monoclonal antibody (mAb) directed against the human T-lymphocyte receptor, CD4; and (d) the anti-CD20 antibody rituximab.

Following a four hour incubation at 37° C., the supernatants were collected and counted on a gamma counter (Isodata Gamma Counter, Packard Instruments). The counts per minute were plotted as a function of antibody concentration and cell cytotoxicity curves from each of the four donors were analyzed using varying concentrations of antibodies. The percentage lysis of target cells was calculated as follows:

$$\% Lysis = \frac{\begin{matrix}sample Release ( C P M ) - \\ spontaneous release ( C P M )\end{matrix}}{\begin{matrix}maximum release ( C P M ) - \\ spontaneous release C P M )\end{matrix}} \times 100 \%$$

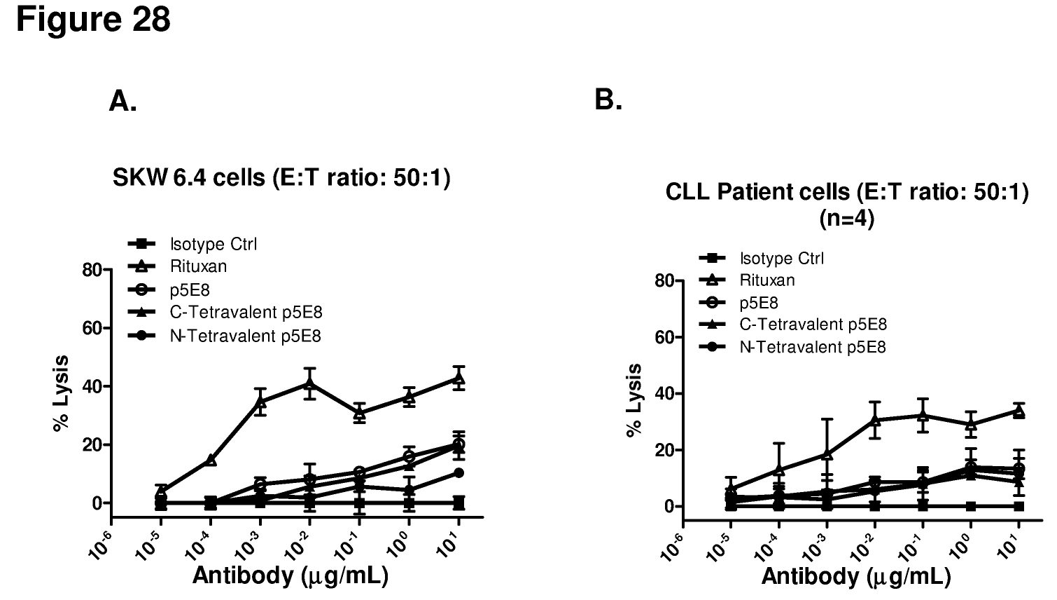

Maximum release was defined as the counts detected following exposure to the Triton X-100. These studies demonstrated that although the tetravalent p5E8 antibodies had weak ADCC activity, this activity was comparable to the parental p5E8 antibody in both SKW6.4 cells and also CLL patient PBMCs (FIG. 28).

### Example 11

### Tetravalent p5E8 Antibodies Display Similar CDC Activities as the Parental p5E8 Antibodies

To determine whether complement fixing activity remained similar between p5E8 and tetravalent p5E8 antibodies Complement Dependent Cytotoxicity (CDC) assays were performed using either SKW6.4 cells or CLL B cells from four individual CLL donors.

Target B cells (SKW6.4 or CLL cells) were washed once and plated in ATCC RPMI 1640 media containing 10% FBS at a volume of 1×10⁴ cells/well. Normal human serum complement was added at a final concentration of 16.7%. Five controls were used. These included (a) target cells in medium; (b) target cells in the presence of 1% TRITON-X®-100; (c) CE9.1, a macaque/human chimeric IgG1 monoclonal antibody (mAb) directed against the human T-lymphocyte receptor, CD4; (d) the anti-CD20 antibody rituximab and (e) the anti-CD52 antibody, alemtuzumab.

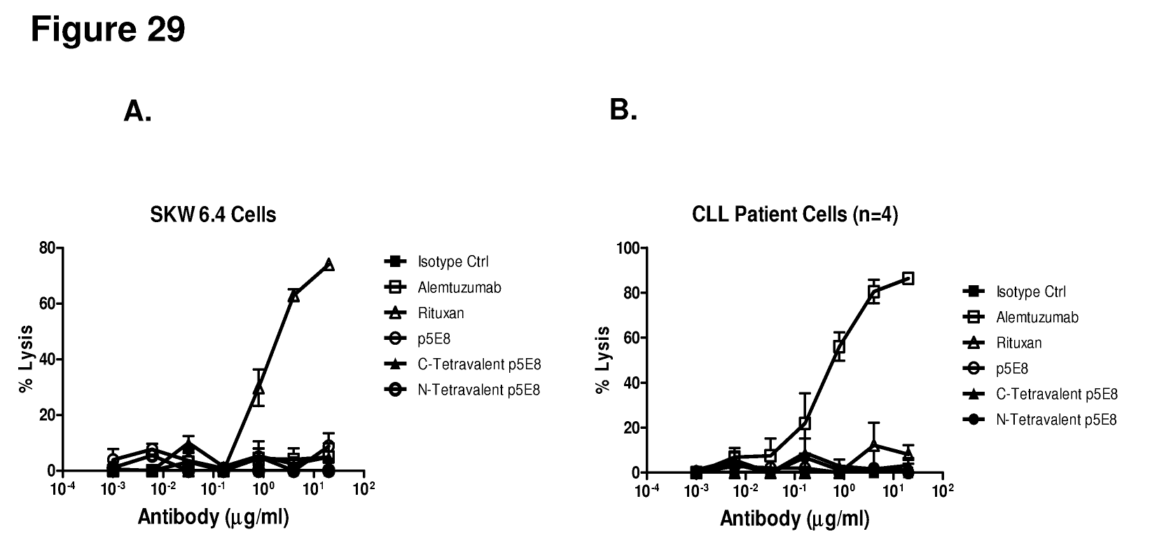

Following a four hour incubation at 37° C., cell viability was measured using a Promega Cell Titer Glo Cell Viability Assay. Results were plotted as a function of antibody concentration and cell cytotoxicity curves from each of the four donors were analyzed using varying concentrations of antibodies. This analysis demonstrated that, similar to the parental p5E8 molecule, no CDC activity was detected with the tetravalent p5E8 antibodies in either CD23+ B cell lines or in CLL patient samples (FIG. 29).

### Example 12

### Tetravalent p5E8 Antibodies Increase Apoptosis in Immortalized Human B Cell Lines

The primary mechanism of action attributed to p5E8 is apoptotic cell death. This cell death is dependent on cross-linking of CD23 via the Fc region of p5E8. Since it was hypothesized that the tetravalent p5E8 may enhance the cross-linking of CD23 on the cell surface, apoptosis assays were performed to quantitate this effect.

Briefly 1×10⁶ SKW6.4 CD23+ B lymphoma cells or MEC1 CLL cells were incubated in a range of concentrations of test antibodies diluted in culture media containing 2% FBS for 45 minutes on ice. Cells were then transferred into culture media containing 5% FBS in the presence or absence of secondary antibody (Goat anti-human IgG, Fc specific, Jackson Immunoresearch, 109-006-098) to provide a source of cross-linker to ligate CD23 antibodies bound on the surface of B cells. Cells were incubated at 37° C./5% CO2 for up to 48 hours and subsequently harvested for apoptosis analysis using the following assays (i)-(iv).

(i) Annexin V Assay: 1×10⁶ cells were washed in PBS prior to staining with Annexin-V-PE and 7AAD as per manufacturer's instructions (BD Pharmingen 559763). Samples were analyzed using a BD FACSArray and data analyzed using GraphPad Prism software.
(ii) Caspase 3/PARP cleavage ELISA: 1×10⁶ cells were washed in PBS prior to preparing a protein lysate. Cleared lysates were incubated on 96 well plates coated with antibodies specific for the cleaved forms of either Caspase 3 or PARP. Binding was measured using anOpt EIA kit and absorbance read at 450 nM as per manufacturer's instructions (BD Pharmingen, Caspase3 552592, PARP 550903)
(iii) Western Blotting: 1×10⁷ cells treated with test antibodies at a concentration of 10 μg/ml as described above and were lysed in RIPA buffer containing protease and phosphatase inhibitors. Protein concentrations were determined using a Bio-Rad DC Protein Assay kit and 25 μg of each lysate was separated on a NUPAGE Bis-TRIS gels (Invitrogen, NP0321-2) prior to transfer to nitrocellulose membrane. Membranes were probed for either PARP (BD Pharmingen, 556494), XIAP (Cell signaling, 2045), cleaved and full length caspase 3 (Imgenex IMG-144A), or GADPH (Santa Cruz Biotechnology sc-25778) as a loading control using standard western blotting procedures.
(iv) ApoBrdU Assay: 1×10⁶ cells were washed in PBS prior to permeabilization and fixation. Cells were stained with anti-BrdU using an Apo-BrdU staining kit (BD Pharmingen 556405) as per manufacturers instructions prior to capturing data on a BD FACS Caliber. FACS data was analyzed using GraphPad Prism software.

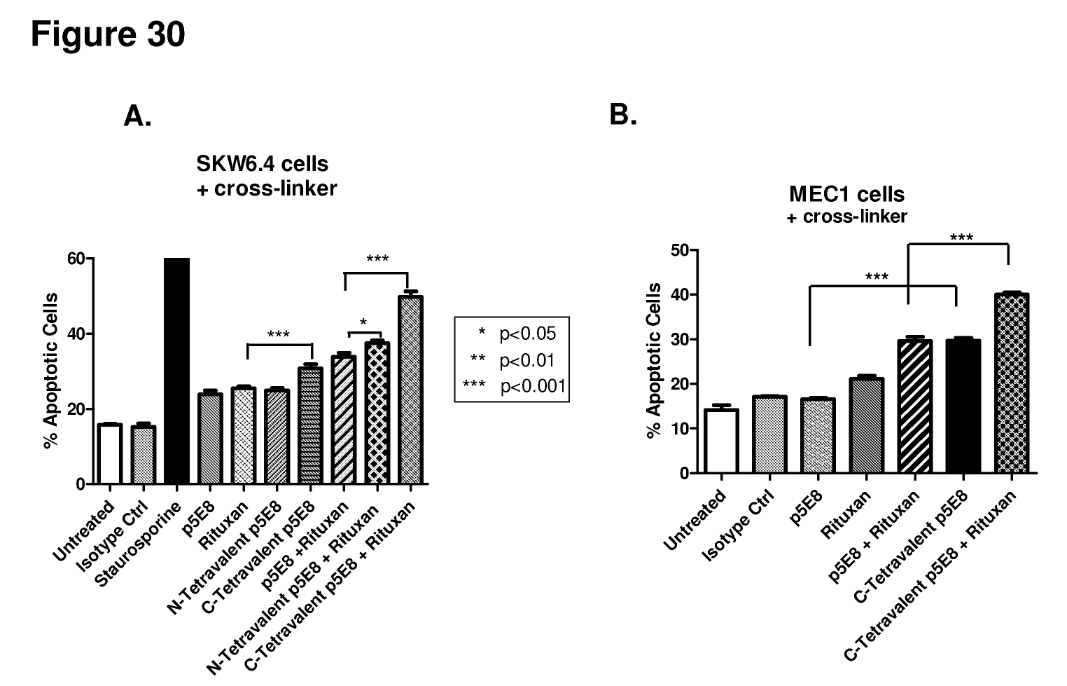

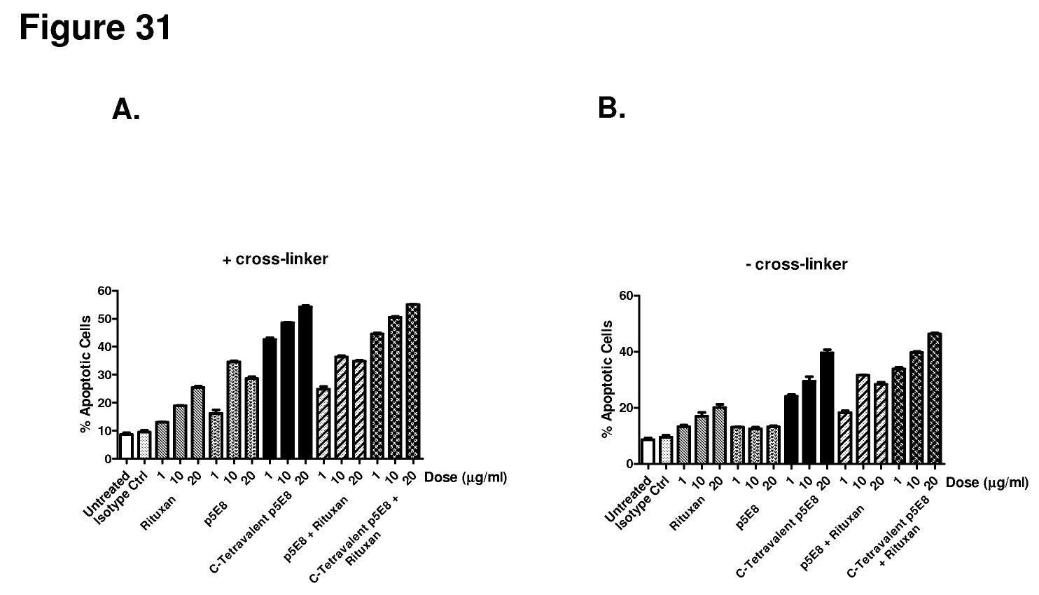

Annexin V apoptosis assays were used to demonstrate that the C-tetravalent p5E8 induces more apoptosis as a single agent than the bivalent p5E8 antibody in both SKW6.4 cells and MEC1 cells when exogenous secondary antibody was used as a source of cross-linking agent (FIG. 30). Both the N- and C-tetravalent p5E8 molecules displayed enhanced rates of apoptosis when used in combination with rituximab when compared to the parental p5E8 antibody. FIG. 31A demonstrates that the apoptosis induced by the C-tetravalent p5E8 is dose-dependent. The rates of apoptosis observed with the C-tetravalent p5E8 was greatly enhanced compared to the parental p5E8 antibody and was also significantly greater than the combination of p5E8 and rituximab.

To determine whether tetravalent p5E8 was sufficient to induce apoptosis without the addition of an exogenous source of cross-linking agent, cells were incubated with antibodies without the addition of secondary antibody and apoptosis scored using an annexin V assay. FIG. 31B demonstrates that apoptosis can be induced by the C-tetravalent p5E8 in the absence of exogenous secondary antibody used to cross-link p5E8. Significant apoptosis was observed with the C-tetravalent p5E8 was compared to the parental antibody, which has negligible activity in the absence of cross-linking. This data demonstrates that tetravalent p5E8 antibody can override the requirement of exogenous cross-linking of p5E8 to induce apoptosis.

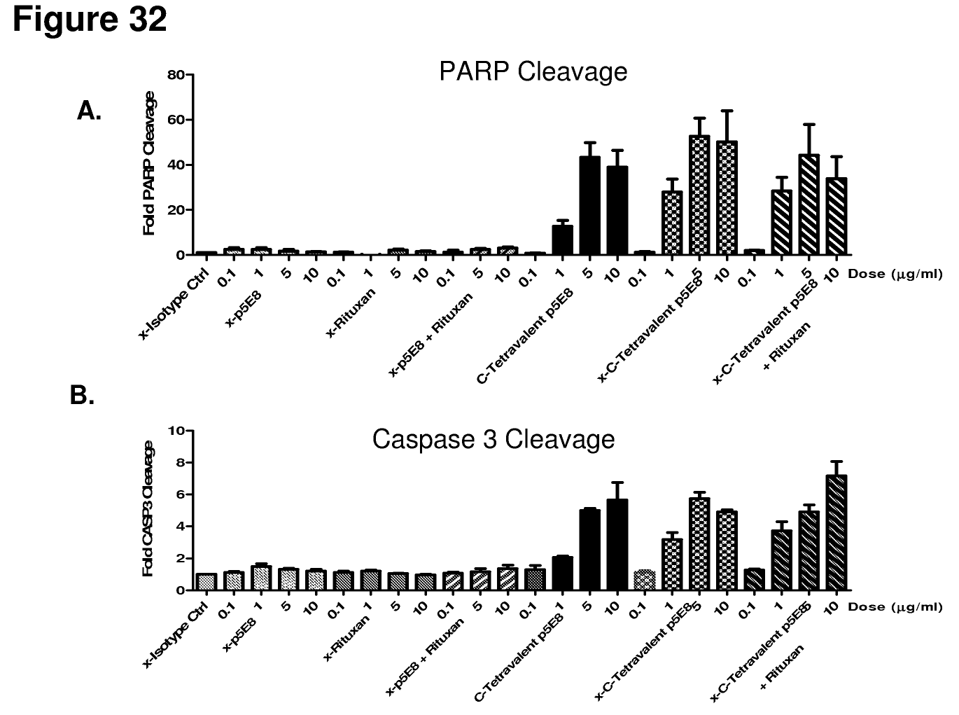

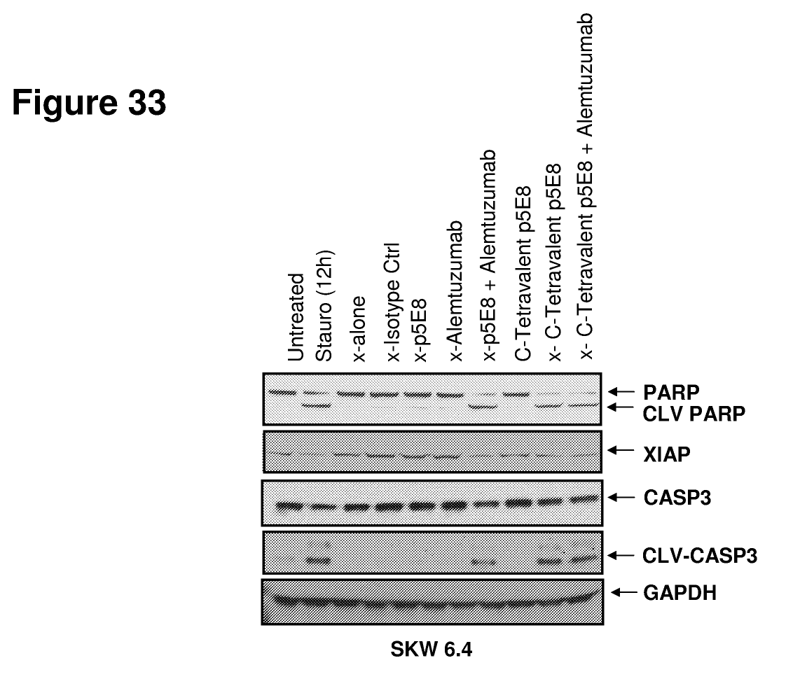

To determine whether other apoptotic characteristics could be detected in cells treated with tetravalent p5E8 downstream apoptotic signaling proteins were examined following antibody treatment. FIG. 32 demonstrates that the C-tetravalent p5E8 results in the cleavage and activation of both (A) PARP and (B) caspase 3, respectively, in a dose-dependent manner in SKW6.4 cells. Apoptotic activity was seen with the C-tetravalent p5E8 in the absence of a cross-linking secondary antibody. Western blotting analysis was also performed to determine whether apoptotic markers could be observed in treated cells. These results confirmed the ELISA results above demonstrating cleavage of PARP and caspase 3 following treatment of cells with tetravalent p5E8 as a single agent (FIG. 33).

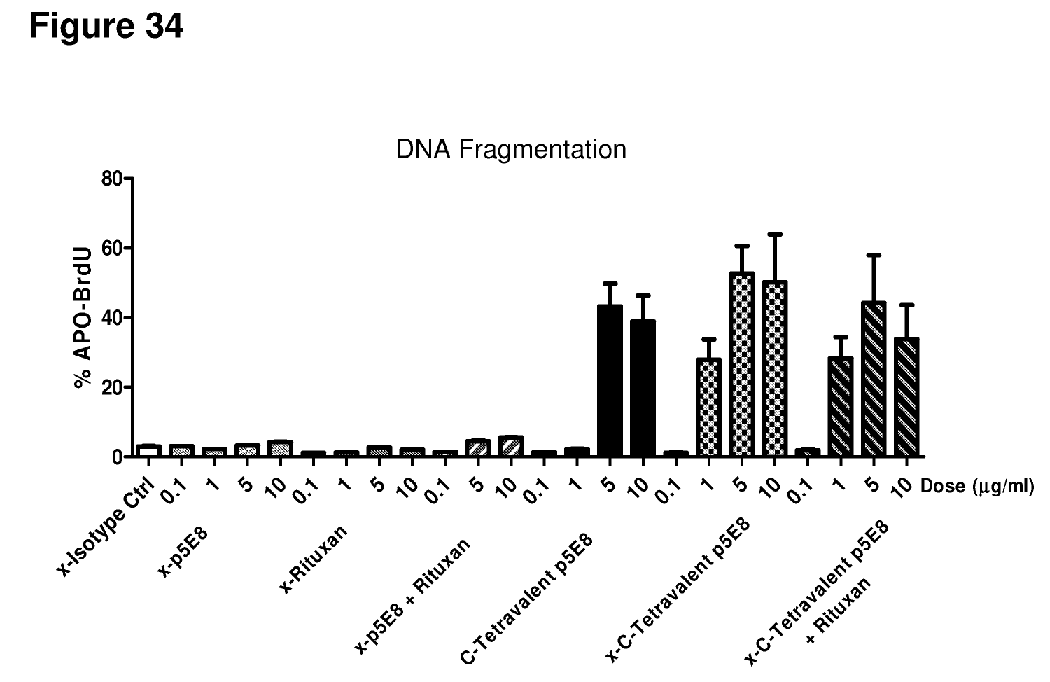

The end stage of the apoptotic cascade is the fragmentation of DNA which leads to the ultimate destruction of the cell. To determine whether tetravalent p5E8 results in elevated levels of DNA fragmentation an ApoBrdU assay was performed to quantitate this late apoptotic response. These results demonstrate that the C-tetravalent p5E8 results in higher levels of DNA fragmentation (FIG. 34). As described above, activity was observed in the absence of secondary antibody cross-linking. In these assays additional activity could be observed at sub optimal doses of antibody when secondary antibody was added demonstrating that cross-linking of the tetravalent p5E8 molecule can further enhance its function. Taken together these results confirm the enhanced apoptotic activity of tetravalent p5E8 in CD23+ B cell lines.

### Example 13

### Tetravalent p5E8 Induces Apoptosis in a Higher Proportion of CLL B Cells Treated Ex Vivo

To determine whether the enhanced apoptotic effects observed in cell lines would hold true for primary CLL B cells, annexin V experiments were performed using PBMC isolated from ten individual CLL patient blood samples. Briefly, human peripheral blood mononuclear cells (PBMC) were purified from heparinized whole blood by standard Ficoll-paque separation (Sigma, Histopaque 1077-1). 1×10⁶ PBMC were incubated in a range of concentrations of test antibodies diluted in culture media containing 2% FBS for 45 minutes on ice. Cells were then transferred into culture media containing 5% FBS in the presence of secondary antibody (Goat anti human IgG, Fc specific, Jackson Immunoresearch, 109-006-098) to provide a source of cross-linker to ligate CD23 antibodies bound on the surface of B cells. Cells were incubated at 37° C./5% CO2 for up to 48 hours and subsequently harvested for apoptosis analysis using an annexin V Assay as described above.

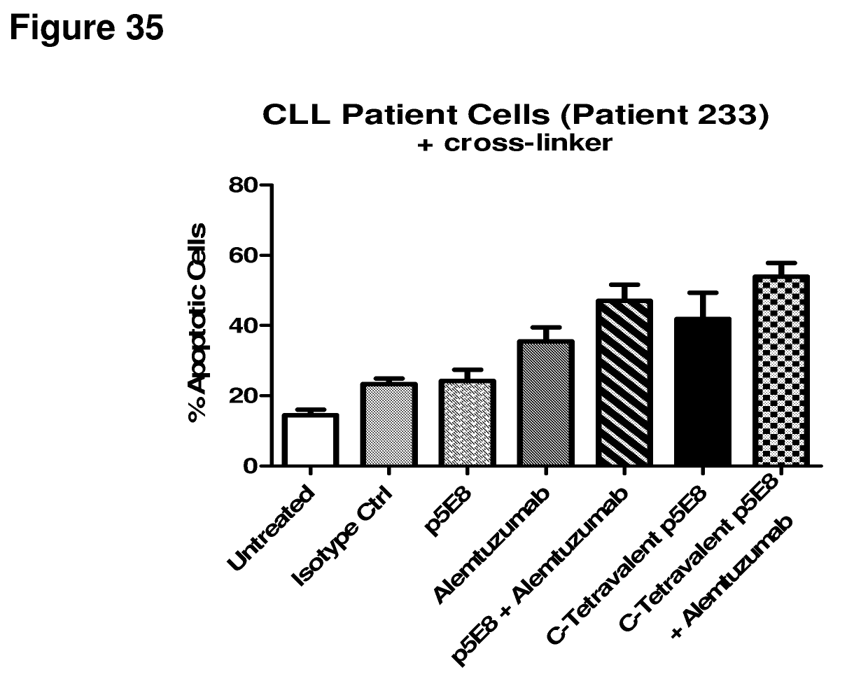

An example from one patient (ID #233) is shown in FIG. 35 demonstrating enhanced apoptosis with the tetravalent p5E8 as a single agent or in combination with the anti-CD52 antibody alemtuzumab. Similar results were observed with 8 additional patient samples and these results are summarized in Table 11. These results demonstrate that tetravalent p5E8 antibody treatment of CLL patient B cells results in an increased number of patients responding to antibody treatment compared to the parental p5E8 antibody. Of interest, it was also noted that one patient failed to respond to either tetravalent p5E8 or alemtuzumab in these in vitro assays. When both antibodies were combined apoptosis was observed in this patient sample which had failed to respond to any single agent treatment or the combination of alemtuzumab with the parental p5E8. This suggests that the elevated apoptosis observed with the tetravalent p5E8 may have clinical benefit in patients who currently don't respond will to existing therapies.

TABLE 11
Apoptotic response of CLL B cells to tetravalent p5E8.

| | CLL Patient Response |
|---|---|
| Drug Treatment | (Apoptosis) |
| p5E8 | 5/10 |
| Alemtuzumab | 9/10 |
| p5E8 + Alemtuzumab | 9/10 |
| Tetravalent p5E8 | 9/10 |
| Tetravalent p5E8 + Alemtuzumab | 10/10 |

### Example 14

### Pharmacokinetics of Tetravalent p5E8 Antibody

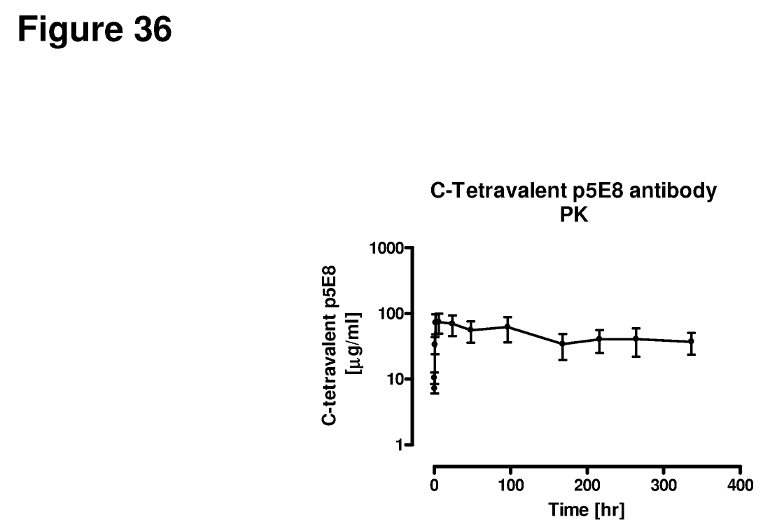

Pharmacokinetic studies were performed in CB17 SCID mice to address the stability and serum half-life of the tetravalent p5E8 molecule. Mice were maintained in accordance with the Biogen Idec Institutional Animal Care and Use Committee, and city, state, and federal guidelines for the humane treatment and care of laboratory animals. A single bolus injection of 10 mg/kg (1 mg/ml) of C-tetravalent p5E8 antibody diluted in phosphate-buffered saline (PBS) was administered intraperitoneally into male CB17-scid mice. Mice were sacrificed at 0, 0.25, 0.5, 1, 2, 6, 24, 48, 96, 168, 216, 264, and 336 hours post-injection. Serum samples were prepared for analysis to quantify levels of the C-tetravalent p5E8 antibody using the modified version of the assay as described in Example 3. The samples were diluted in DAB supplemented with 5% normal mouse serum (Jackson ImmunoResearch 015-000-120), and the detection reagent was an Eu-labeled mouse anti-Human Fc antibody (Perkin Elmer 1244-330) used at a final concentration of 250 ng/ml. Quantitation was performed by using Excel's TREND function in comparison to a standard curve of purified C-tetravalent p5E8 antibody. Results of the pharmacokinetic study are shown in FIG. 36. C-tetravalent p5E8 has an apparent long elimination half-life consistent with that observed for normal IgGs (7-14 days).

### Example 15

### In Vivo Efficacy of Tetravalent P5E8 Antibody in a Disseminated Lymphoma Model

Animal model studies (e.g., murine xenograft models) may be conducted to determine whether tetravalent p5E8 antibodies have enhanced in vivo efficacy. Briefly, 4×10⁶ SKW6.4 CD23+ B cells are injected into the tail vein of CB-17 SCID mice at day 0. Starting at day 3 twice weekly intra-peritoneal injections of antibody or isotype control are performed until all control animals are lost due to excessive tumor burden. The parental, bivalent p5E8 antibody may be tested side by side with the tetravalent p5E8 antibody either as a single agent or in combination with rituximab to determine whether increased valency results in prolonged disease free survival. It is predicted that the tetravalent p5E8 will have enhanced efficacy as both a single agent and in combination with rituximab when compared to the parental p5E8. Xenograft studies may also be performed to address the in vivo relevance of cross-linking CD23. In humans FcγRIIIa is the FcγR which has been demonstrated to be important for the response of patients to rituximab and the mouse equivalent, FcγRIV, is expressed on macrophages. To address the role that cross-linking may have on p5E8 function additional studies will be performed examining the efficacy of the parental p5E8 and tetravalent p5E8 in CB-17 SCID mice where the macrophage population has been depleted with clodronate encapsulated liposomes. It is predicted that where the bivalent p5E8 antibody will show efficacy in the macrophage expressing mice, little efficacy will be observed when macrophages are depleted. In contrast, the tetravalent p5E8 antibody is predicted to show efficacy in both the presence and absence of macrophages.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A multivalent CD23 binding molecule comprising more than two CD23 binding moieties, wherein said binding molecule specifically crosslinks at least two distinct human CD23 molecules on the surface of an immune cell, thereby inducing apoptosis of the immune cell.

2. The binding molecule of claim 1, wherein said binding molecule comprises at least four binding moieties.

3. The binding molecule of claim 1, wherein said binding molecule binds to FcγR.

4. The binding molecule of claim 1, wherein said binding molecule induces CD23-mediated caspase-3 and PARP cleavage.

5. The binding molecule of claim 1, wherein said binding molecule induces apoptosis to a greater extent than an equimolar amount of an antibody dimer formed by crosslinking of two bivalent CD23 monoclonal antibodies with a cross-linker.

6. The binding molecule of claim 5, wherein the binding molecule induces apoptosis 1.5 fold or more, 2-fold or more, 3-fold or more, 4-fold or more, 5-fold or more, 6 fold or more, 7-fold or more, 8 fold or more, 9-fold or more, 10-fold or more, and 15-fold or more than an equimolar amount of an antibody dimer formed by crosslinking of two bivalent CD23 monoclonal antibodies with a cross-linker.

7. The binding molecule of claim 5, wherein the equimolar amount is an amount selected from the group consisting of 1 ug/ml or more, 2 ug/ml or more, 5 ug/ml or more, 10 ug/ml or more, 15 ug/ml or more, and 20 ug/ml or more.

8. The binding molecule of claim 1, wherein three, four, five, six, seven, eight, nine, ten, or more CD23 molecules are crosslinked by said multivalent CD23 binding molecule.

9. The binding molecule of claim 1, wherein apotosis of the immune cell is determined by an apoptotic assay selected from the group consisting of: a PARP cleavage assay, a TUNEL assay, a Caspase cleavage assay, and a mitochondrial membrance permeabilization assay.

10. The binding molecule of claim 1, wherein the multivalent binding molecule is not crosslinked with a second multivalent binding molecule by a crosslinker.

11. The binding molecule of claim 1, wherein the multivalent binding molecule is crosslinked with a second multivalent binding molecule by a crosslinker.

12. The binding molecule of claim 5, wherein the cross-linker is an antibody which binds to the anti-CD23 antibody.

13. The binding molecule of claim 5, wherein the cross-linker is an engineered disulfide bond.

14. The binding molecule of claim 1, which binds to human CD23.

15. The binding molecule of claim 1, wherein two of said binding moieties are binding sites derived from an antibody selected from the group consisting of a 5E8 antibody, a 6G5 antibody, a 2C8 antibody, a B3B11 antibody, and a 3G12 antibody.

16. The binding molecule of claim 1, wherein the cell is a CLL cell.

17. A tetravalent CD23 antibody molecule comprising four CD23 binding moieties and two heavy chain polypeptides, wherein two of said binding moieties are provided by an IgG antibody and two of said binding moieties are provided by two scFv molecules linked or fused to said IgG antibody.

18. The binding molecule of claim 17, wherein said IgG antibody comprises light chain (VL) and heavy chain (VH) variable domains derived from a 5E8 antibody.

19. The binding molecule of claim 18, wherein said VL domain of said IgG antibody comprises the amino acid sequence of SEQ ID NO:97 and said VH domain of said IgG antibody comprises the amino acid sequence of SEQ ID NO:89.

20. The binding molecule of claim 17, wherein one or both of said scFv molecules comprise a light chain (VL) and a heavy chain (VH) variable domain derived from a 5E8 antibody.

21. The binding molecule of claim 20, wherein said VL domain of said scFv molecules comprise the amino acid sequence of SEQ ID NO:97 and said VH domain of said scFv molecules comprise the amino acid sequence of SEQ ID NO:89.

22. The binding molecule of claim 17, wherein one or both of said scFv molecules is a stabilized scFv molecule having a T50 of greater than 55° C.

23. The binding molecule of claim 17, wherein one or both of said scFv molecules is a stabilized scFv molecule having a T50 that is at least 2° C.-10° C. higher than that of a conventional 5E8 scFv molecule (SEQ ID NO:6 or SEQ ID NO:8).

24. The binding molecule of claim 22, wherein said stabilized scFv molecule is a stabilized scFv molecule comprising the amino acid sequence of pIEH252 (SEQ ID NO:10) or pIEH246 (SEQ ID NO:12).

25. The binding molecule of claim 17, wherein one or both of said scFv molecules is fused to said IgG antibody via a Gly/Ser linker.

26. The binding molecule of claim 25, wherein said Gly/Ser linker is a (Gly₄Ser)₅ or Ser(Gly₄Ser)₃ linker.

27. The binding molecule of claim 17, wherein said scFv molecules are linked or fused to said IgG antibody via the VL domain of said scFv molecules.

28. The binding molecule of claim 27, wherein the scFv molecule is of the orientation VH→(Gly4Ser)ₙ linker→VL, and wherein n is 3, 4, 5, or 6.

29. The binding molecule of claim 17, wherein said scFv molecules are linked or fused to said IgG antibody via the VH domain of said scFv molecules.

30. The binding molecule of claim 29, wherein the scFv molecule is of the orientation VL→(Gly4Ser)ₙ linker→VH, and wherein n is 3, 4, 5 or 6.

31. The binding molecule of claim 17, wherein one or both of said scFv molecules is fused to a heavy chain of said IgG antibody to form one or both of the heavy chain polypeptides of said binding molecule.

32. The binding molecule of claim 31, wherein one of said scFv molecules is linked or fused to a first heavy chain of said IgG antibody and one of said scFv molecules is linked or fused to a second heavy chain of said IgG antibody.

33. The binding molecule of claim 31, wherein one or both of said scFv molecules are linked or fused to the N-terminus of said first and second heavy chains of said IgG antibody.

34. The binding molecule of claim 32, wherein the light chains of said IgG antibody comprise the light chain sequence of SEQ ID NO: 4; and wherein the heavy chain polypeptides of said binding molecule comprise the amino acid sequence of SEQ ID NO:14 or SEQ ID NO:18.

35. The binding molecule of claim 17, wherein said binding molecule is produced by the cell line 4F4 deposited on Sep. 26, 2008 as ATCC Deposit No. PTA-9530.

36. The binding molecule of claim 32, wherein one or both of said scFv molecules are fused to the C-terminus of said first and second heavy chains of said IgG antibody.

37. The binding molecule of claim 36, wherein the light chains of said IgG antibody comprise the sequence of SEQ ID NO: 4 (p5E8) and wherein the heavy chain polypeptides of said binding molecule comprise the amino acid sequence of SEQ ID NO:2, SEQ ID NO:16, or SEQ ID NO:20.

38. The binding molecule of claim 17, wherein said binding molecule is produced by the cell line 1E2 deposited on Sep. 26, 2008 as ATCC Deposit No. PTA-9531.

39. The binding molecule of claim 17, wherein one or both of said scFv molecules is linked or fused to a light chain of said IgG antibody.

40. The binding molecule of claim 39, wherein one of said scFv molecules is linked or fused to a first light chain of said IgG antibody and one of said scFv molecules is linked or fused to a second light chain of said IgG antibody.

41. The binding molecule of claim 39, wherein one or both of said scFv molecules are linked or fused to the N-terminus of said first and second light chains of said IgG antibody.

42. The binding molecule of claim 17, wherein said IgG antibody comprises heavy chain constant domains of the human IgG4 isotype.

43. The binding molecule of claim 17, wherein said IgG antibody comprises heavy chain constant domains of the human IgG1 isotype.

44. The antibody molecule of claim 42 or 43, wherein the heavy chain constant regions of said IgG antibody are afucosylated.

45. The binding molecule of claim 22, which is essentially resistant to aggregation when produced at commercial scale.

46. A stabilized scFv molecule having binding specificity for CD23, wherein the stabilized scFv molecule has a T50 of greater than 55° C.

47. The stabilized scFv molecule of claim 46, which comprises at least four stabilizing mutations as compared to a conventional scFv molecule, wherein said mutations are independently selected from the group consisting of:
a) substitution of an amino acid (e.g., glutamic acid) at Kabat position 6 of VH, e.g., with glutamine;
b) substitution of an amino acid (e.g., asparagine) at Kabat position 32 of VH, e.g., with serine;
c) substitution of an amino acid (e.g., serine) at Kabat position 49 of VH, e.g., with glycine or alanine;
d) substitution of an amino acid (e.g., proline) at Kabat position 56 of VH, e.g., with a histidine;
e) substitution of an amino acid (e.g., glutamic acid) at Kabat position 72 of VH, e.g., with aspartic acid;
f) substitution of an amino acid (e.g., valine) at Kabat position 50 of VL, e.g., with serine, glutamic acid, or aspartic acid;
g) substitution of an amino acid (e.g., valine) at Kabat position 75 of VL, e.g., with isoleucine; and
h) substitution of an amino acid (e.g., phenylalanine) at Kabat position 83 of VL, e.g., with serine, alanine, glycine, or threonine.

48. The stabilized scFv molecule of claim 46, wherein the scFv molecule is derived from a 5E8 antibody.

49. The stabilized scFv molecule of claim 46, wherein the scFv molecule comprises at least one of the combinations of mutations selected from the group consisting of:
(i) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with glutamic acid, and (f) an amino acid (e.g., phenylalanine) at Kabat position 83 of VL, e.g., with alanine;
(ii) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with aspartic acid, and (f) an amino acid (e.g., phenylalanine) at Kabat position 83 of VL, e.g., with alanine;
(iii) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with glutamic acid, and (f) an amino acid (e.g., valine) at Kabat position 75 of VL, e.g., with isoleucine;
(iv) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with glutamic acid, and (e) an amino acid (e.g., phenylalanine) at Kabat position 83 of VL, e.g., with alanine;
(v) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with aspartic acid, and (e) an amino acid (e.g., phenylalanine) at Kabat position 83 of VL, e.g., with alanine;
(vi) substitution of: (a) an amino acid (e.g., glutamic acid) at Kabat position 6 of VH, e.g., with glutamine, (b) an amino acid (e.g., asparagine) at Kabat position 32 of VH, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with serine, and (f) an amino acid (e.g., valine) at Kabat position 75 of VL, e.g., with isoleucine;
(vii) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., proline) at Kabat position 56 of VH, e.g., with histidine, (d) an amino acid (e.g., valine) at Kabat position 50 of VL, e.g., with serine, and (e) an amino acid (e.g., phenylalanine) at Kabat position 83 of VL, e.g., with alanine;
(viii) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., proline) at Kabat position 56 of VH, e.g., with histidine; and (e) an amino acid (e.g., valine) at Kabat position 50 of VL, e.g., with aspartic acid;
(ix) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., proline) at Kabat position 56 of VH, e.g., with histidine, (e) an amino acid (e.g., valine) at Kabat position 50 of VL, e.g., with serine, (f) an amino acid (e.g., valine) at Kabat position 75 of VL, e.g., with isoleucine, and (g) an amino acid (e.g., phenylalanine) at Kabat position 83 of VL, e.g., with alanine;
(x) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with aspartic acid, and (f) an amino acid (e.g., phenylalanine) at Kabat position 83 of VL, e.g., with alanine;
(xi) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with glutamic acid, and (f) an amino acid (e.g., phenylalanine) at Kabat position 83 of VL, e.g., with alanine;
(xii) substitution of: (a) an amino acid (e.g., glutamic acid) at Kabat position 6 of VH, e.g., with glutamine, (b) an amino acid (e.g., serine) at Kabat position 49 of VH, e.g., with glycine, (c) an amino acid (e.g., glutamic acid) at Kabat position 72 of VH, e.g., with aspartic acid, (d) an amino acid (e.g., valine) at Kabat position 50 of VL, e.g., with glutamic acid, and (f) an amino acid (e.g., phenylalanine) at Kabat position 83 of VL, e.g., with alanine; and
(xiii) substitution of: (a) an amino acid (e.g., glutamic acid) at Kabat position 6 of VH, e.g., with glutamine, (b) an amino acid (e.g., asparagine) at Kabat position 32, e.g., with serine, (c) an amino acid (e.g., serine) at Kabat position 49 of VH, e.g., with glycine, (d) an amino acid (e.g., glutamic acid) at Kabat position 72 of VH, e.g., with aspartic acid, (e) an amino acid (e.g., valine) at Kabat position 50 of VL, e.g., with serine, and (f) an amino acid (e.g., phenylalanine) at Kabat position 83 of VL, e.g., with alanine.

50. A binding molecule comprising at least one stabilized scFv molecule of claim 46.

51. A composition comprising the binding molecule of any one of claims 1, 17, and 46 and a carrier.

52. A nucleic acid molecule encoding a binding molecule of any one of claims 1, 17, and 46.

53. A host cell comprising the nucleic acid molecule of claim 52.

54. A method of manufacturing a CD23 binding molecule comprising culturing the host cell of claim 53 under conditions such that the binding molecule is expressed, and isolating the binding molecule.

55. The method of claim 54, wherein the host cell is cultured at commercial scale and wherein at least 5 mg of the stabilized binding molecule is produced for every liter of the host cell culture medium.

56. The method of claim 54, wherein the host cell is cultured at commercial scale and wherein at least 50 mg of the stabilized binding molecule is produced for every liter of the host cell culture medium.

57. A CD23 binding molecule manufactured according to the method of claim 54.

58. The binding molecule of claim 57, where the isolated binding molecule is resistant to aggregation when the host cell is cultured commercial scale.

59. A method of decreasing tumor growth or metastasis in a human subject comprising administering to the subject an effective amount of a binding molecule of any one of claims 1, 17, and 46.

60. The method of claim 57, wherein the human subject has chronic lymphocytic leukemia.

61. The method of claim 60, further comprising the administration of at least one additional agent.

62. The method of claim 61, wherein the at least one additional agent is one or more agents selected from the group consisting of fludarabine, cyclophosphamide and rituximab.

63. The method of claim 61, wherein the additional agents are fludarabine, cyclophosphamide and rituximab.

64. A method of inducing CD23-mediated caspase-3 or PARP cleavage in a cancer cell bearing CD23, comprising contacting the cancer cell with a multivalent CD23 binding molecule comprising at least four CD23 binding moieties, wherein said binding molecule binds to FcγR and specifically crosslinks at least two distinct human CD23 molecules on the surface of the cancer cell.

65. The method of claim 64, wherein the cancer cell is a CLL cell.

66. The method of claim 64, wherein said cleavage is induced to a greater extent than an equimolar amount of an antibody dimer formed by crosslinking of two bivalent CD23 monoclonal antibodies with a cross-linker.

67. The method of claim 66, wherein the binding molecule induces cleavage 1.5 fold or more, 2-fold or more, 3-fold or more, 4-fold or more, 5-fold or more, 6 fold or more, 7-fold or more, 8 fold or more, 9-fold or more, 10-fold or more, and 15-fold or more than the equimolar amount of antibody dimer.

68. The method of claim 67, wherein the equimolar amount is an amount selected from the group consisting of 1 ug/ml or more, 2 ug/ml or more, 5 ug/ml or more, 10 ug/ml or more, 15 ug/ml or more, and 20 ug/ml or more.

69. The method of claim 64, wherein three, four, five, six, seven, eight, nine, ten, or more CD23 molecules are crosslinked by said multivalent CD23 binding molecule.

70. The method of claim 66, wherein the cross-linker is an antibody which binds to the anti-CD23 antibody.

71. The method of claim 66, wherein the cross-linker is an engineered disulfide bond.

72. The method of claim 64, wherein said binding molecule is a tetravalent CD23 antibody molecule comprising four CD23 binding moieties and two heavy chain polypeptides, wherein two of said binding moieties are provided by an IgG antibody and two of said binding moieties are provided by two scFv molecules linked or fused to said IgG antibody.


SEQUENCE LISTING (107 sequences)

SEQ ID NO: 1 - DNA, 2115 bp, Artificial Sequence
  Synthetic sequence
gaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggcgggggtggatccggtggagggggctccggcggtggcgggtccgacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatgttgcatccagtttgcaaagtggggtcccatcaaggttcagcggcagtggatctgggacagagttcactctcaccgtcagcagcctgcagcctgaagattttgcgacttattactgtctacaggtttatagtacccctcggacgttcggccaagggaccaaggtggaaatcaaaggcggtggcgggtccggtgggggtggctccgggggcggtggctccgaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcatga

SEQ ID NO: 2 - protein, 704 aa, Artificial Sequence
  Synthetic sequence
EVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIKGGGGSGGGGSGGGGSEVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSS

SEQ ID NO: 3 - DNA, 645 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatgttgcatccagtttgcaaagtggggtcccatcaaggttcagcggcagtggatctgggacagagttcactctcaccgtcagcagcctgcagcctgaagattttgcgacttattactgtctacaggtttatagtacccctcggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 4 - protein, 214 aa, Artificial Sequence
  Synthetic sequence
DIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 720 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatgttgcatccagtttgcaaagtggggtcccatcaaggttcagcggcagtggatctgggacagagttcactctcaccgtcagcagcctgcagcctgaagattttgcgacttattactgtctacaggtttatagtacccctcggacgttcggccaagggaccaaggtggaaatcaaaggcggtggcgggtccggtgggggtggctccgggggcggtggctccgaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctca

SEQ ID NO: 6 - protein, 240 aa, Artificial Sequence
  Synthetic sequence
DIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIKGGGGSGGGGSGGGGSEVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSS

SEQ ID NO: 7 - DNA, 720 bp, Artificial Sequence
  Synthetic sequence
gaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcaggcggtggcgggtccggtgggggtggctccgggggcggtggctccgacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatgttgcatccagtttgcaaagtggggtcccatcaaggttcagcggcagtggatctgggacagagttcactctcaccgtcagcagcctgcagcctgaagattttgcgacttattactgtctacaggtttatagtacccctcggacgttcggccaagggaccaaggtggaaatcaaa

SEQ ID NO: 8 - protein, 240 aa, Artificial Sequence
  Synthetic sequence
EVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSSGGGGSGGGGSGGGGSDIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIK

SEQ ID NO: 9 - DNA, 720 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcagcaactactacatggactgggtccgccaggctccagggcaggggctggagtgggtcggccgtattagtagtagtggtgatcccacatggtacgcagactccgtgaagggcagattcaccatctccagagacaacgccaagaacacactgtttcttcaaatgaacagcctgagagctgaggacacggctgtctattactgtgcgagcttgactacagggtctgactcctggggccagggagtcctggtcaccgtctcctca

SEQ ID NO: 10 - protein, 240 aa, Artificial Sequence
  Synthetic sequence
DIQMTQSPSSLSASVGDRVTITCRASQDIRYYLNWYQQKPGKAPKLLIYSASSLQSGVPSRFSGSGSGTEFTLTVSSLQPEDAATYYCLQVYSTPRTFGQGTKVEIKGGGGSGGGGSGGGGSEVQLVQSGGGLAKPGGSLRLSCAASGFRFTFSNYYMDWVRQAPGQGLEWVGRISSSGDPTWYADSVKGRFTISRDNAKNTLFLQMNSLRAEDTAVYYCASLTTGSDSWGQGVLVTVSS

SEQ ID NO: 11 - DNA, 723 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcaataactactacatggactgggtccgccaggctccagggcaggggctggagtgggtcggccgtattagtagtagtggtgatcacacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcatga

SEQ ID NO: 12 - protein, 240 aa, Artificial Sequence
  Synthetic sequence
DIQMTQSPSSLSASVGDRVTITCRASQDIRYYLNWYQQKPGKAPKLLIYSASSLQSGVPSRFSGSGSGTEFTLTVSSLQPEDAATYYCLQVYSTPRTFGQGTKVEIKGGGGSGGGGSGGGGSEVQLVQSGGGLAKPGGSLRLSCAASGFRFTFNNYYMDWVRQAPGQGLEWVGRISSSGDHTWYADSVKGRFTISRENAKNTLFLQMNSLRAEDTAVYYCASLTTGSDSWGQGVLVTVSS

SEQ ID NO: 13 - DNA, 2142 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcagcaactactacatggactgggtccgccaggctccagggcaggggctggagtgggtcggccgtattagtagtagtggtgatcccacatggtacgcagactccgtgaagggcagattcaccatctccagagacaacgccaagaacacactgtttcttcaaatgaacagcctgagagctgaggacacggctgtctattactgtgcgagcttgactacagggtctgactcctggggccagggagtcctggtcaccgtctcctcaggcggtggagggtccggtggagggggctctggagggggcggttcagggggcggtggatccgggggaggtggctccgaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 713 aa, Artificial Sequence [sequence omitted]

SEQ ID NO: 15 - DNA, 2115 bp, Artificial Sequence
  Synthetic sequence
gaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggcgggggtggatccggtggagggggctccggcggtggcgggtccgacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcagcaactactacatggactgggtccgccaggctccagggcaggggctggagtgggtcggccgtattagtagtagtggtgatcccacatggtacgcagactccgtgaagggcagattcaccatctccagagacaacgccaagaacacactgtttcttcaaatgaacagcctgagagctgaggacacggctgtctattactgtgcgagcttgactacagggtctgactcctggggccagggagtcctggtcaccgtctcctcatga

SEQ ID NO: 16 - protein, 704 aa, Artificial Sequence
  Synthetic sequence
EVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQSPSSLSASVGDRVTITCRASQDIRYYLNWYQQKPGKAPKLLIYSASSLQSGVPSRFSGSGSGTEFTLTVSSLQPEDAATYYCLQVYSTPRTFGQGTKVEIKGGGGSGGGGSGGGGSEVQLVQSGGGLAKPGGSLRLSCAASGFRFTFSNYYMDWVRQAPGQGLEWVGRISSSGDPTWYADSVKGRFTISRDNAKNTLFLQMNSLRAEDTAVYYCASLTTGSDSWGQGVLVTVSS

SEQ ID NO: 17 - DNA, 2142 bp, Artificial Sequence
  Synthetic sequence
gacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcaataactactacatggactgggtccgccaggctccagggcaggggctggagtgggtcggccgtattagtagtagtggtgatcacacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcaggcggtggagggtccggtggagggggctctggagggggcggttcagggggcggtggatccgggggaggtggctccgaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 18 - protein, 713 aa, Artificial Sequence
  Synthetic sequence
DIQMTQSPSSLSASVGDRVTITCRASQDIRYYLNWYQQKPGKAPKLLIYSASSLQSGVPSRFSGSGSGTEFTLTVSSLQPEDAATYYCLQVYSTPRTFGQGTKVEIKGGGGSGGGGSGGGGSEVQLVQSGGGLAKPGGSLRLSCAASGFRFTFNNYYMDWVRQAPGQGLEWVGRISSSGDHTWYADSVKGRFTISRENAKNTLFLQMNSLRAEDTAVYYCASLTTGSDSWGQGVLVTVSSGGGGSGGGGSGGGGSGGGGSGGGGSEVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 2115 bp, Artificial Sequence
  Synthetic sequence
gaggtgcagctggtggagtctgggggcggcttggcaaagcctggggggtccctgagactctcctgcgcagcctccgggttcaggttcaccttcaataactactacatggactgggtccgccaggctccagggcaggggctggagtgggtctcacgtattagtagtagtggtgatcccacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggcgggggtggatccggtggagggggctccggcggtggcgggtccgacatccagatgacccagtctccatcttccctgtctgcatctgtaggggacagagtcaccatcacttgcagggcaagtcaggacattaggtattatttaaattggtatcagcagaaaccaggaaaagctcctaagctcctgatctatagcgcatccagtttgcaaagtggggtcccatcaaggttcagcggcagtggatctgggacagagttcactctcaccgtcagcagcctgcagcctgaagatgccgcgacttattactgtctacaggtttatagtacccctcggacgttcggccaagggaccaaggtggaaatcaaaggcggtggcgggtccggtgggggtggctccgggggcggtggctccgaggtgcagctggtgcagtctgggggcggcttggcaaagcctggggggtccctgagactctcctgcgcagcctccgggttcaggttcaccttcaataactactacatggactgggtccgccaggctccagggcaggggctggagtgggtcggccgtattagtagtagtggtgatcacacatggtacgcagactccgtgaagggcagattcaccatctccagagagaacgccaagaacacactgtttcttcaaatgaacagcctgagagctgaggacacggctgtctattactgtgcgagcttgactacagggtctgactcctggggccagggagtcctggtcaccgtctcctcatga

SEQ ID NO: 20 - protein, 704 aa, Artificial Sequence
  Synthetic sequence
EVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQSPSSLSASVGDRVTITCRASQDIRYYLNWYQQKPGKAPKLLIYSASSLQSGVPSRFSGSGSGTEFTLTVSSLQPEDAATYYCLQVYSTPRTFGQGTKVEIKGGGGSGGGGSGGGGSEVQLVQSGGGLAKPGGSLRLSCAASGFRFTFNNYYMDWVRQAPGQGLEWVGRISSSGDHTWYADSVKGRFTISRENAKNTLFLQMNSLRAEDTAVYYCASLTTGSDSWGQGVLVTVSS

SEQ ID NO: 21 - protein, 19 aa, Artificial Sequence
  Synthetic peptide
MGWSLILLFLVAVATRVLS

SEQ ID NO: 22 - protein, 22 aa, Artificial Sequence
  Synthetic peptide
MDMRVPAQLLGLLLLWLPGARC

SEQ ID NO: 23 - DNA, 44 bp, Artificial Sequence
  Synthetic oligonucleotide P5E8-VL01F
cgctggtggtgccgttctatagccatagtgacatccagatgacc

SEQ ID NO: 24 - DNA, 21 bp, Artificial Sequence
  Synthetic oligonucleotide P5E8-VL01R
gtggtcgactttgatttccac

SEQ ID NO: 25 - DNA, 47 bp, Artificial Sequence
  Synthetic oligonucleotide P5E8-VH01F
cgctggtggtgccgttctatagccatagtgaggtgcagctggtggag

SEQ ID NO: 26 - DNA, 24 bp, Artificial Sequence
  Synthetic oligonucleotide P5E8-VH01R
gtggtcgactgaggagacggtgac

SEQ ID NO: 27 - DNA, 52 bp, Artificial Sequence
  Synthetic oligonucleotide P5E8-Leader01
ggcatatgaaaaaactgctgttcgcgattccgctggtggtgccgttctatag

SEQ ID NO: 28 - DNA, 33 bp, Artificial Sequence
  Synthetic oligonucleotide VH_E6Q
gaggtgcagctggtgcagtctgggggcggcttg

SEQ ID NO: 29 - DNA, 33 bp, Artificial Sequence
  Synthetic oligonucleotide VH_L11SDG
gagtctgggggcggcrrcgcaaagcctgggggg

SEQ ID NO: 30 - DNA, 37 bp, Artificial Sequence
  Synthetic oligonucleotide VH_A12VK_K13QER
gtctgggggcggcttgrhgmagcctggggggtccctg

SEQ ID NO: 31 - DNA, 34 bp, Artificial Sequence
  Synthetic oligonucleotide VH_N32S
gttcaggttcaccttcagcaactactacatggac

SEQ ID NO: 32 - DNA, 35 bp, Artificial Sequence
  Synthetic oligonucleotide VH_D35bHSN
caataactactacatgmrctgggtccgccaggctc

SEQ ID NO: 33 - DNA, 33 bp, Artificial Sequence
  Synthetic oligonucleotide VH_Q43KR
cgccaggctccagggarggggctggagtgggtc

SEQ ID NO: 34 - DNA, 34 bp, Artificial Sequence
  Synthetic oligonucleotide VH_S49GA
gggctggagtgggtcgsccgtattagtagtagtg

SEQ ID NO: 35 - DNA, 34 bp, Artificial Sequence
  Synthetic oligonucleotide VH_I51M
gagtgggtctcacgtatgagtagtagtggtgatc

SEQ ID NO: 36 - DNA, 39 bp, Artificial Sequence
  Synthetic oligonucleotide VL_Q37A
ggtattatttaaattggtatgcccagaaaccaggaaaag

SEQ ID NO: 37 - DNA, 36 bp, Artificial Sequence
  Synthetic oligonucleotide VH_D55GS
gtattagtagtagtggtrgccccacatggtacgcag

SEQ ID NO: 38 - DNA, 32 bp, Artificial Sequence
  Synthetic oligonucleotide VH_P56STD
gtagtagtggtgatrvcacatggtacgcagac

SEQ ID NO: 39 - DNA, 32 bp, Artificial Sequence
  Synthetic oligonucleotide VH_W58YN
gtggtgatcccacawactacgcagactccgtg

SEQ ID NO: 40 - DNA, 36 bp, Artificial Sequence
  Synthetic oligonucleotide VH_E72DN
gattcaccatctccagaracaacgccaagaacacac

SEQ ID NO: 41 - DNA, 35 bp, Artificial Sequence
  Synthetic oligonucleotide VH_A74S
catctccagagagaacagcaagaacacactgtttc

SEQ ID NO: 42 - DNA, 33 bp, Artificial Sequence
  Synthetic oligonucleotide VH_F79YSV
gccaagaacacactgkhccttcaaatgaacagc

SEQ ID NO: 43 - DNA, 35 bp, Artificial Sequence
  Synthetic oligonucleotide VH_Q81E
gaacacactgtttcttgaaatgaacagcctgagag

SEQ ID NO: 44 - DNA, 36 bp, Artificial Sequence
  Synthetic oligonucleotide VH_A84G
caaatgaacagcctgagaggcgaggacacggctgtc

SEQ ID NO: 45 - DNA, 32 bp, Artificial Sequence
  Synthetic oligonucleotide VH_V89AGT
gagctgaggacacggctrsctattactgtgcg

SEQ ID NO: 46 - DNA, 34 bp, Artificial Sequence
  Synthetic oligonucleotide VH_S94RK
gtctattactgtgcgargttgactacagggtctg

SEQ ID NO: 47 - DNA, 34 bp, Artificial Sequence
  Synthetic oligonucleotide VH_V107T
ctcctggggccagggaaccctggtcaccgtctcc

SEQ ID NO: 48 - DNA, 35 bp, Artificial Sequence
  Synthetic oligonucleotide VH_L108AGS
ctggggccagggagtckscgtcaccgtctcctcag

SEQ ID NO: 49 - DNA, 33 bp, Artificial Sequence
  Synthetic oligonucleotide VH_T110VS
cagggagtcctggtckycgtctcctcagtcgac

SEQ ID NO: 50 - DNA, 33 bp, Artificial Sequence
  Synthetic oligonucleotide VL_L11G
cagtctccatcttccggctctgcatctgtaggg

SEQ ID NO: 51 - DNA, 32 bp, Artificial Sequence
  Synthetic oligonucleotide VL_V15AGS
ccctgtctgcatctrscggggacagagtcacc

SEQ ID NO: 52 - DNA, 32 bp, Artificial Sequence
  Synthetic oligonucleotide VL_V19L
ctgtaggggacagactgaccatcacttgcagg

SEQ ID NO: 53 - DNA, 32 bp, Artificial Sequence
  Synthetic oligonucleotide VL_I21L
ggggacagagtcaccctgacttgcagggcaag

SEQ ID NO: 54 - DNA, 33 bp, Artificial Sequence
  Synthetic oligonucleotide VL_T22S
gacagagtcaccatcagctgcagggcaagtcag

SEQ ID NO: 55 - DNA, 37 bp, Artificial Sequence
  Synthetic oligonucleotide VL_D28SG
gcagggcaagtcagrgcattaggtattatttaaattg

SEQ ID NO: 56 - DNA, 33 bp, Artificial Sequence
  Synthetic oligonucleotide VL_R30SL
gcaagtcaggacattmkctattatttaaattgg

SEQ ID NO: 57 - DNA, 32 bp, Artificial Sequence
  Synthetic oligonucleotide VL_K39A
aattggtatcagcaggccccaggaaaagctcc

SEQ ID NO: 58 - DNA, 34 bp, Artificial Sequence
  Synthetic oligonucleotide VL_K45ER
ccaggaaaagctcctsrcctcctgatctatgttg

SEQ ID NO: 59 - DNA, 37 bp, Artificial Sequence
  Synthetic oligonucleotide VL_V50n
ctaagctcctgatctatnnkgcatccagtttgcaaag

SEQ ID NO: 60 - DNA, 33 bp, Artificial Sequence
  Synthetic oligonucleotide VL_L54R
ctatgttgcatccagtcgccaaagtggggtccc

SEQ ID NO: 61 - DNA, 34 bp, Artificial Sequence
  Synthetic oligonucleotide VL_V58S
cagtttgcaaagtgggtccccatcaaggttcagc

SEQ ID NO: 62 - DNA, 34 bp, Artificial Sequence
  Synthetic oligonucleotide VL_E70D
cagtggatctgggacagacttcactctcaccgtc

SEQ ID NO: 63 - DNA, 32 bp, Artificial Sequence
  Synthetic oligonucleotide VL_V75I
gagttcactctcaccatcagcagcctgcagcc

SEQ ID NO: 64 - DNA, 30 bp, Artificial Sequence
  Synthetic oligonucleotide VL_P80AG
cagcagcctgcagrgcgaagattttgcgac

SEQ ID NO: 65 - DNA, 32 bp, Artificial Sequence
  Synthetic oligonucleotide VL_F83AGST
ctgcagcctgaagatrscgcgacttattactg

SEQ ID NO: 66 - DNA, 33 bp, Artificial Sequence
  Synthetic oligonucleotide VL_T85D
cctgaagattttgcggactattactgtctacag

SEQ ID NO: 67 - DNA, 33 bp, Artificial Sequence
  Synthetic oligonucleotide VL_L89AQ
gcgacttattactgtsmrcaggtttatagtacc

SEQ ID NO: 68 - DNA, 33 bp, Artificial Sequence
  Synthetic oligonucleotide VL_R96LY
gtttatagtacccctmwaacgttcggccaaggg

SEQ ID NO: 69 - DNA, 32 bp, Artificial Sequence
  Synthetic oligonucleotide VL_F98W
gtacccctcggacgtggggccaagggaccaag

SEQ ID NO: 70 - DNA, 33 bp, Artificial Sequence
  Synthetic oligonucleotide VL_I106AGS
gggaccaaggtggaakscaaaggcggtggcggg

SEQ ID NO: 71 - DNA, 32 bp, Artificial Sequence
  Synthetic oligonucleotide VH_L45C
gctccagggcaggggtgcgagtgggtctcacg

SEQ ID NO: 72 - DNA, 32 bp, Artificial Sequence
  Synthetic oligonucleotide VL_F98C
gtacccctcggacgtgcggccaagggaccaag

SEQ ID NO: 73 - DNA, 35 bp, Artificial Sequence
  Synthetic oligonucleotide VH_D101C
cttgactacagggtcttgctcctggggccagggag

SEQ ID NO: 74 - DNA, 32 bp, Artificial Sequence
  Synthetic oligonucleotide VL_L46C
ggaaaagctcctaagtgcctgatctatgttgc

SEQ ID NO: 75 - DNA, 34 bp, Artificial Sequence
  Synthetic oligonucleotide VH_S102C
gactacagggtctgactgctggggccagggagtc

SEQ ID NO: 76 - DNA, 32 bp, Artificial Sequence; identical to SEQ ID NO: 74
  Synthetic oligonucleotide VL_L46C

SEQ ID NO: 77 - DNA, 34 bp, Artificial Sequence
  Synthetic oligonucleotide VH_G44C
caggctccagggcagtgcctggagtgggtctcac

SEQ ID NO: 78 - DNA, 36 bp, Artificial Sequence
  Synthetic oligonucleotide VL_Q100C
cctcggacgttcggctgcgggaccaaggtggaaatc

SEQ ID NO: 79 - DNA, 62 bp, Artificial Sequence
  Synthetic oligonucleotide 078F3
gttgctacgcgtggcggtggatccgggggaggtggctccgaggtgcagctggtggagtctgg

SEQ ID NO: 80 - DNA, 37 bp, Artificial Sequence
  Synthetic oligonucleotide 078-R3
gttaacagatcttcatttacccggagacagggagagg

SEQ ID NO: 81 - DNA, 43 bp, Artificial Sequence
  Synthetic oligonucleotide 078-F1
gttgctacgcgtgtcctgtccgacatccagatgacccagtctc

SEQ ID NO: 82 - DNA, 84 bp, Artificial Sequence
  Synthetic oligonucleotide 078-R4
tcccccggatccaccgccccctgaaccgccccctccagagccccctccaccggaccctccaccgcctgaggagacggtgaccag

SEQ ID NO: 83 - DNA, 62 bp, Artificial Sequence
  Synthetic oligonucleotide C-23VL-F
agagagggatccggtggagggggctccggcggtggcgggtccgacatccagatgacccagtc

SEQ ID NO: 84 - DNA, 36 bp, Artificial Sequence
  Synthetic oligonucleotide C-23VH-R
agagagagatcttcatgaggagacggtgaccaggac

SEQ ID NO: 85 - protein, 122 aa, Unknown
  Non-human primate
QLQLQESGPGVVKPSETLSLTCAVSGGSVSSSNWWTWIRQPPGKGLEWIGRISGSGGATNYNPSLKSRVIISQDTSKNQFSLNLNSVTAADTAVYYCARDWAQIAGTTLGFWGQGVLVTVSS

SEQ ID NO: 86 - protein, 6 aa, Unknown
  Non-human primate
SSNWWT

SEQ ID NO: 87 - protein, 17 aa, Unknown
  Non-human primate
RISGSGGATNYNPSLKS

SEQ ID NO: 88 - protein, 12 aa, Unknown
  Non-human primate
DWAQIAGTTLGF

SEQ ID NO: 89 - protein, 118 aa, Unknown
  Non-human primate
EVQLVESGGGLAKPGGSLRLSCAASGFRFTFNNYYMDWVRQAPGQGLEWVSRISSSGDPTWYADSVKGRFTISRENAKNTLFLQMNSLRAEDTAVYYCASLTTGSDSWGQGVLVTVSS

SEQ ID NO: 90 - protein, 7 aa, Unknown
  Non-human primate
FNNYYMD

SEQ ID NO: 91 - protein, 17 aa, Unknown
  Non-human primate
RISSSGDPTWYADSVKG

SEQ ID NO: 92 - protein, 7 aa, Unknown
  Non-human primate
LTTGSDS

SEQ ID NO: 93 - protein, 95 aa, Unknown
  Non-human primate
QSAPTQPPSVSGSPGQSVTISCTGTSDDVGGYNYVSWYQHHPGKAPKLMIYDVAKRASGVSDRFSGSKSGNTAYCCSYTTSSTLLFGRGTRLTVL

SEQ ID NO: 94 - protein, 14 aa, Unknown
  Non-human primate
TGTSDDVGGYNYVS

SEQ ID NO: 95 - protein, 7 aa, Unknown
  Non-human primate
DVAKRAS

SEQ ID NO: 96 - protein, 10 aa, Unknown
  Non-human primate
CSYTTSSTLL

SEQ ID NO: 97 - protein, 107 aa, Unknown
  Non-human primate
DIQMTQSPSSLSASVGDRVTITCRASQDIRYYLNWYQQKPGKAPKLLIYVASSLQSGVPSRFSGSGSGTEFTLTVSSLQPEDFATYYCLQVYSTPRTFGQGTKVEIK

SEQ ID NO: 98 - protein, 11 aa, Unknown
  Non-human primate
RASQDIRYYLN

SEQ ID NO: 99 - protein, 10 aa, Unknown
  Non-human primate
VASSLQSPRT

SEQ ID NO: 100 - protein, 9 aa, Unknown
  Non-human primate
LQVYSTPRT

SEQ ID NO: 101 - protein, 5 aa, Artificial Sequence
  Synthetic gly-ser connecting peptide
GGGGS

SEQ ID NO: 102 - protein, 20 aa, Artificial Sequence
  Synthetic gly-ser connecting peptide
GGGGSGGGGSGGGGSGGGGS

SEQ ID NO: 103 - protein, 6 aa, Artificial Sequence
  Synthetic gly-ser connecting peptide
SGGGGS

SEQ ID NO: 104 - protein, 15 aa, Artificial Sequence
  Synthetic gly-ser connecting peptide
GGGGSGGGGSGGGGS

SEQ ID NO: 105 - protein, 25 aa, Artificial Sequence
  Synthetic gly-ser connecting peptide
GGGGSGGGGSGGGGSGGGGSGGGGS

SEQ ID NO: 106 - protein, 16 aa, Artificial Sequence
  Synthetic gly-ser connecting peptide
SGGGGSGGGGSGGGGS

SEQ ID NO: 107 - protein, 25 aa, Artificial Sequence
  Synthetic linker peptide
GGGGSGGGGSGGGGSGGGGSGGGAS